(19) United States
(12) Patent Application Publication
(10) Pub. No.: US 2025/0270314 A1
(43) Pub. Date: Aug. 28, 2025

(54) ANTI-CDH17 MONOCLONAL AND BISPECIFIC ANTIBODIES AND USES THEREOF

(71) Applicants: TAVOTEK BIOTECH (SUZHOU) LTD, Suzhou (CN); HANGZHOU UNOGEN BIOTECH, LTD., Hangzhou, Zhejiang (CN)

(72) Inventors: YuanTing CHEN, Lower Gwynedd, PA (US); Mark L. CHIU, Lower Gwynedd, PA (US); Brian WHITAKER, Lower Gwynedd, PA (US); Mark A. TORNETTA, Lower Gwynedd, PA (US); Man-Cheong FUNG, Lower Gwynedd, PA (US); Pu PU, Hong Kong (CN); ChengDong LIU, Hong Kong (CN); Kenneth Cheung KWONG, Yardley, PA (US); Ao YU, Shanghai (CN); Glenn Mark ANDERSON, Eagleville, PA (US)

(21) Appl. No.: 18/292,697

(22) PCT Filed: Aug. 2, 2022
(86) PCT No.: PCT/US2022/074408

§ 371 Date: Jan. 26, 2024

Related U.S. Application Data
(60) Provisional application No. 63/228,200, filed on Aug. 2, 2021.

(51) Int. Cl. C07K 16/28

(52) U.S. Cl. CPC C07K 16/28; C07K 16/2809; C07K 2317/31

(57) ABSTRACT

The present disclosure relates to the application of antibodies and bispecific antibodies targeting human Cadherin-17 and CD3 as an effective and tissue-specific treatment of CDH17-mediated diseases or disorders such as breast, lung, pancreatic, gastric, and other CDH17 overexpressed cancers. The bispecific antibodies may contain masking domains to minimize systemic toxicity. The masking domains are fused via protease-cleavable linkers to the Fab domains targeting human Cadherin-17 and CD3. The unmasking of the shielded bispecific antibodies occurs predominantly by proteases and enzymes in the tumor microenvironment. The application of such bispecific antibodies thereby minimizes systemic toxicity and expands the therapeutic index.

### CROSS-REFERENCE TO RELATED APPLICATION

This application claims priority to U.S. Provisional Application No. 63/228,200, filed Aug. 2, 2021, the entire contents of which are hereby incorporated by reference.

### SEQUENCE LISTING

This application contains a Sequence Listing, which has been submitted electronically in ST.26 format and is hereby incorporated by reference in its entirety.

### FIELD OF THE DISCLOSURE

The present disclosure relates to biomedicine, particularly, antibodies and bispecific antibodies directed against a CDH17 Family protein such as cell adhesion molecule Cadherin-17, nucleic acids encoding such antibodies, methods for preparing such antibodies, and methods for the treatment of diseases such as cancers mediated by Cadherin-17 expression and/or activity.

### BACKGROUND OF THE DISCLOSURE

Stomach and liver cancers are among the most lethal worldwide with over half of the incidences being diagnosed in China. Although surgical resection of early-stage gastric cancer offers an excellent chance of cure, the prognosis for advanced gastric cancer (GC) remains poor with a 5-year survival rate of less than 40%. Unfortunately, and very often, most of these cancers are asymptomatic and clinically detected at advanced stages. Without effective treatment, these patients die shortly after the diagnosis or relapse after salvage therapies. For patients with late stage and metastatic GC, the overall 5-year survival ranges from 5% to 15%. Lymph node metastasis (LNM) is generally responsible for the high mortality rate of GC.

Cadherin-17 (CDH17) has been identified as a biomarker used to predict poor prognosis for GC. CDH17 is also known as liver-intestine (LI)-cadherin or intestinal peptide-associated transporter (HPT-1). CDH17 is present in fetal liver and the gastrointestinal tract, exhibiting elevated expression during embryogenesis. CDH17 localizes to the basolateral domain of hepatocytes and enterocytes, where it mediates intercellular adhesion in a Ca²⁺-dependent manner to maintain tissue integrity in epithelial tissue. Cadherins are a superfamily of calcium dependent cell adhesion molecules that preferentially interact with themselves to connect cells. Although CDH17 belongs to the cadherin superfamily via its intercellular conjunction activity, CDH17 is distinct from E, N, and P cadherins since it retains its adhesive function without interacting with other cytoplasmic components.

In normal human tissues, CDH17 is exclusively expressed on intestinal human epithelial cells. In colon cancer, CDH17 is expressed at low levels in primary tumors or in regional lymph node metastases, as well as in poorly differentiated colon cancer tumors. However, CDH17 is overexpressed in advanced colorectal cancer liver metastasis, where it correlates with poor prognosis. CDH17 is not detected in normal tissues of kidney, lung, liver, brain, adrenal gland, or skin. CDH17 expression has been found in gastric cancer (Ko, Chu et al. 2004, Dong, Yu et al. 2007, Altree-Tacha, Tyrrell et al. 2017), pancreatic cancer, colorectal cancer (Su, Yuan et al. 2008, Tian, Han et al. 2018), and hepatocellular carcinoma (Takamura, Sakamoto et al. 2003, Wong, Luk et al. 2003) as shown in FIG. 1. Because of the limited distribution in normal tissues, CDH17 is a useful marker for the identification of cancer.

The Cadherin-17 structure is characterized as having an extracellular domain with 7 cadherin domains, a single hydrophobic transmembrane domain, and a short C-terminal cytoplasmic tail as shown in FIG. 2 (Lin, Zhang et al. 2014). Cadherin-17 is characterized by an amino-terminal duplication of the first two cadherin repeats, so that seven (instead of five) repeats are present in the ectodomain. Moreover, the cytoplasmic domains of CDH17 is truncated and lacks the two armadillo protein binding domains, which are typical of classical cadherins (van Roy 2014). Only one human CDH17 isoform is known, GenBank Accession No. NM_004063. Cadherin-17 has the UniProt accession number Q12864. The mouse Cadherin-17 orthologue has the UniProt accession number Q9R100 and shows 76% identity to the human Cadherin-17.

Not only a static intercellular adhesion molecule, CDH17 plays an important role during the process of invasion or metastasis in colorectal, liver, and gastric cancers. High expression of CDH17 is associated with high metastatic potential, positive lymph node metastasis, and short overall survival in gastric cancer (GC) patients (Lee, Nam et al. 2010). CDH17 can regulate the activity of integrin-Ras/Raf/MEK/ERK pathway for cell proliferation in GC (Lin, Zhang et al. 2014). CDH17 is also present in neuroendocrine tumors (NETs) that constitute a diverse group of epithelial neoplasms that can arise in a wide variety of anatomical sites (Snow, Mangray et al. 2015). Most NETs occur in the gastrointestinal, pancreatic, and bronchopulmonary systems. Pancreatic neuroendocrine tumors (PanNETs) and small intestine neuroendocrine tumors (SINETs) are the second most common malignancies in the pancreas and small intestine. PanNETs and SINETs share similar morphologies, and both frequently metastasize to the liver.

CDH17 is an oncogene that is involved in tumor invasion and metastasis. Recent studies suggested that CDH17 is also an oncogene in gastric cancer; knockdown of endogenous CDH17 can reduce cancer cell proliferation and increase apoptosis partly via downregulating Wnt/beta-catenin signaling (Qiu, Zhang et al. 2013). CDH17 binds to α2β1 subunit of integrin to promote cancer as shown in FIG. 3. Thus, increased CDH17 expression of tumors enhance integrin activation and signaling, which correlated with a moderately increased adhesion and proliferation of cancer cells. Integrins are transmembrane receptors that facilitate cell-cell and cell-extracellular matrix (ECM) adhesion. Upon ligand binding, integrins activate signal transduction pathways that mediate cellular signals such as regulation of the cell cycle, organization of the intracellular cytoskeleton, and movement of new receptors to the cell membrane. The presence of integrins allows rapid and flexible responses to events at the cell surface. Integrins work alongside other proteins such as cadherins, the immunoglobulin superfamily cell adhesion molecules, selectins and syndecans, to mediate cell-cell and cell-matrix interactions.

An antibody against the RGD motif of CDH17 significantly increases survival and reduction of liver metastasis in mice injected intrasplenically with colon cancer cells (Bartolome, Aizpurua et al. 2018). CDH17 can also activate the I kappa B kinase (IKK) complex, which subsequently phosphorylates the IκB-α. Then the phosphorylated IκB-α undergoes proteasome-dependent decomposition, which releases the heterodimers of p65/p50 into cytoplasm, which are then transferred into the nucleus (FIG. 4). Finally, the p65 binds to its responsive gene and promotes the transcription of downstream proteins including CDH17 C and MMP-9. Inhibition of CDH17 can attenuate the activation of IKK in gastric cancer cells, leading to a concomitant reduction in downstream proteins.

In summary, CDH17 can (1) indirectly affect integrins to stabilize their structure and activity, (2) activate the Ras/Raf/MEK/ERK pathway by up-regulation of cadherin-integrin signaling, (3) retain p53 and p21 at lower levels through activation of Ras/Raf/MEK/ERK pathway, and (4) play an important role in cell proliferation, migration, adhesion, colony formation, cell-cycle, and apoptosis.

Inhibition of CDH17 can reduce proliferation and increase apoptosis of gastric cancer cells both in vitro and in vivo (Li, Yang et al. 2017, Liu, Huang et al. 2019). Loss of CDH17 function suppressed pancreatic cancer cell line Panc02-H7 cell growth in vitro and in orthotopic tumor growth in vivo, resulting in significant life extension. In vitro studies demonstrated that impairing CDH17 inhibited cell proliferation, colony formation, and motility by mechanistically modulating pro- and anti-apoptosis events in pancreatic cancer cells. Since CDH17 can function as an oncogenic molecule critical to PC (pancreatic cancer) growth by regulating tumor apoptosis signaling pathways, anti-CDH17 mAbs could be targeted to develop an anti-PC therapeutic approach. Antibodies against cadherins reduced the proliferation, adhesion, and invasion capacity of metastatic cancer cell lines by inhibiting the activation of a2ß1 integrin. The extended mice survival demonstrates the potential therapeutic effect in cancer metastasis, specifically in colorectal cancer and melanoma (Bartolome, Aizpurua et al. 2018). Since the naked antibodies were not potent enough, CDH17 ADC (antibody-drug conjugate) immunotoxins were made by coupling saporins for targeting of low expression level cancer cells (Kusano-Arai, Iwanari et al. 2018). However, the activity only works well when a cocktail of ADCs is provided.

To have a stronger patient response against tumors that overexpress CDH17, a bispecific antibody (BsAb) that engages the patient's immune cells can provide better tumor control. Bispecific antibodies have been engineered to be capable of engaging multiple targets in solid cancers. CD3+ bispecific T-cell redirection antibody therapeutics bridge T cells to tumor cells (Singh, Dees et al. 2021). In so doing, the cytolytic activity of CD3+ T cells can be redirected towards tumor cells to facilitate their elimination. This synapse is independent of MHC restriction that requires T cell activation after T cell receptor binding to a major histocompatibility complex (MHC) molecule. Such functional BsAb activity cannot be achieved when using a combination of monovalent antibodies. The first bispecific CD3+ T-cell redirector, catumaxomab (RemovabR), was approved by the European Union (EU) for the treatment of malignant ascites in 2009 (EMA 2021). However, this anti-CD3×anti-epithelial cell adhesion molecule (EpCAM) antibody induces off-target hepatotoxicity in patients due to its binding to hepatic macrophages and was later withdrawn from the market (Heiss, Murawa et al. 2010). Later, the Food Drug Administration (FDA) approved the anti-CD3×anti-CD19 bispecific T-cell engager blinatumomab (Blincyto®) for the treatment of patients with Philadelphia chromosome-negative B-cell acute lymphoblastic leukemia (ALL) (Amgen 2020, Amgen 2021, Singh, Dees et al. 2021). Blinatumomab is now approved for the treatment of patients with Philadelphia-negative and -positive relapsed/refractory ALL, as well as those patients with ALL who are in remission but show signs of MRD (Minimal residual disease.

However, blinatumomab use is associated with elevated levels of cytokine release and neurotoxicity (Amgen 2021). Neurotoxicity is very common with Amgen's blinatumomab, occurring in 70% of R/R NHL patients in a phase 2 trial, including 22% with grade 3 or higher toxicity (Viardot, Goebeler et al. 2016). Although blinatumomab is structurally formatted as a bispecific T-cell engager (BiTE)®, additional bispecific antibody designs have been described for CD3+ T-cell redirection (Singh, Dees et al. 2021). Severe neurotoxicity and ICANS are also seen in Roche's mosunetuzumab (CD20×CD3) although to a lower frequency (Sun, Ellerman et al. 2015, Phillips, Olszewski et al. 2020). In another example, ARB202 is an anti-CDH17/CD3 bispecific T-cell engager generated from anti-CDH17 monoclonal ARB102 linked to a CD3-binding scFv. The IgG4-sdFV format could inhibit growth of CDH17 positive tumor in a pancreatic AsPC1 xenograft model. However, since ARB202 has a similar design as the aforementioned BiTE molecules, there can be similar safety concerns.

Nonetheless, numerous reports on the limitations of CD3+ T-cell redirection anti-tumor efficacy point to the recruitment of counterproductive CD3+ T-cell subsets, dose-limiting cytokine storm, the presence of an immunosuppressive tumor microenvironment (TME), T-cell dysfunction and exhaustion due to expression of immune checkpoint molecules, tumor antigen escape, on-target off-tumor toxicity, and suboptimal potency. In May of 2021, Pfizer's Elranatamab (BCMA×CD3) pivotal clinical trial on multiple myeloma was put on clinical hold due to several cases of severe peripheral neuropathy (Biopharmadive.com 2021). In fact, safety rather than efficacy is often a key concern of T-cell redirection as reflected by the relatively limited commercial success of Catumaxomab (voluntary market withdrawal in the US in 2013 and in EU in 2017) and Blinatumomab (2020 global sales of only 379 MM USD despite regulatory approval since 2014) (Amgen 2021, Wikipedia 2021). Thus, increasing the therapeutic index of T-cell redirection to maximize the clinical potential is an unmet medical need and highly desirable.

CD3×CDH17 BsAbs can generate on-target off-tumor toxicities by binding to CDH17 on healthy cells. CDH17 is expressed on tissues of healthy tissues. Thus, T cell destruction of these cells can lead to immune pathology and organ failure with potential fatality as shown in a preclinical mouse study using a CD3×EGFR BsAb (Lutterbuese, Raum et al. 2010). Thus, minimizing the effects of the CD3×CDH17 in normal tissue is critical. The present disclosure provides anti-CDH17 antibodies and bispecific antibodies to address one or more challenges associated with the use of T-cell redirection for CDH17 driven tumor cells.

### SUMMARY OF THE DISCLOSURE

In one aspect, the present disclosure provides an anti-CDH17 antibody or an antigen-binding portion thereof. In some embodiments, the present disclosure provides an anti-CDH17 antibody or an antigen-binding portion thereof comprising a heavy chain variable region comprising three Complementarity Determining Regions (CDRs), designated as HCDR1, HCDR2, and HCDR3, wherein the HCDR1, HCDR2, and HCDR3 are selected from:
SEQ ID NO: 65, SEQ ID NO: 66, and SEQ ID NO: 67; and
SEQ ID NO: 68, SEQ ID NO: 69, and SEQ ID NO: 70; respectively.

In some embodiments, the present disclosure provides an anti-CDH17 antibody or an antigen-binding portion thereof, comprising a light chain variable region comprising three CDRs, designated as LCDR1, LCDR2, and LCDR3, wherein the LCDR1, LCDR2, and LCDR3 are selected from:
SEQ ID NO: 62, SEQ ID NO: 63, and SEQ ID NO: 64; and
SEQ ID NO: 71, SEQ ID NO: 72, and SEQ ID NO: 73; respectively.

In some embodiments, the disclosure provides an anti-CDH17 antibody or an antigen binding fragment thereof, comprising a heavy chain sequence comprising an amino acid sequence with at least 85% identity to any one of SEQ ID NOs: 3 and 5, or an antigen-binding portion thereof, and a light chain sequence comprising an amino acid sequence with at least 85% identity to any one of SEQ ID NOs: 2 and 6, or an antigen-binding portion thereof.

In some embodiments, the disclosure provides an anti-CDH17 antibody or an antigen binding fragment thereof, comprising a heavy chain sequence and a light chain sequence comprising: SEQ ID NOs: 3 and 2; SEQ ID NOs: 4 and 2; SEQ ID NOs: 5 and 6; SEQ ID NOs: 7 and 9; and SEQ ID NOs: 8 and 9; respectively.

In some embodiments, the present disclosure provides an anti-CDH17 antibody or an antigen-binding portion thereof, comprising at least one variable-heavy-chain-only single-domain or an antigen-binding portion thereof, wherein the at least one variable-heavy-chain-only single-domain comprises HCDR1, HCDR2, and HCDR3 selected from:
SEQ ID NO: 74, SEQ ID NO: 75, and SEQ ID NO: 76;
SEQ ID NO: 77, SEQ ID NO: 78, and SEQ ID NO: 79;
SEQ ID NO: 80, SEQ ID NO: 81, and SEQ ID NO: 82;
SEQ ID NO: 77, SEQ ID NO: 83, and SEQ ID NO: 84;
SEQ ID NO: 77, SEQ ID NO: 81, and SEQ ID NO: 85;
SEQ ID NO: 86, SEQ ID NO: 87, and SEQ ID NO: 88;
SEQ ID NO: 86, SEQ ID NO: 89, and SEQ ID NO: 90;
SEQ ID NO: 77, SEQ ID NO: 91, and SEQ ID NO: 92;
SEQ ID NO: 93, SEQ ID NO: 75, and SEQ ID NO: 94;
SEQ ID NO: 77, SEQ ID NO: 95, and SEQ ID NO: 96;
SEQ ID NO: 77, SEQ ID NO: 97, and SEQ ID NO: 98;
SEQ ID NO: 77, SEQ ID NO: 99, and SEQ ID NO: 100;
SEQ ID NO: 86, SEQ ID NO: 101, and SEQ ID NO: 102;
SEQ ID NO: 103, SEQ ID NO: 104, and SEQ ID NO: 105;
SEQ ID NO: 86, SEQ ID NO: 106, and SEQ ID NO: 107;
SEQ ID NO: 108, SEQ ID NO: 109, and SEQ ID NO: 110;
SEQ ID NO: 77, SEQ ID NO: 111, and SEQ ID NO: 112;
SEQ ID NO: 77, SEQ ID NO: 113, and SEQ ID NO: 114;
SEQ ID NO: 115, SEQ ID NO: 116, and SEQ ID NO: 117;
SEQ ID NO: 77, SEQ ID NO: 118, and SEQ ID NO: 119;
SEQ ID NO: 120, SEQ ID NO: 78, and SEQ ID NO: 121;
SEQ ID NO: 77, SEQ ID NO: 101, and SEQ ID NO: 122;
SEQ ID NO: 115, SEQ ID NO: 123, and SEQ ID NO: 124;
SEQ ID NO: 115, SEQ ID NO: 125, and SEQ ID NO: 126;
SEQ ID NO: 115, SEQ ID NO: 127, and SEQ ID NO: 128;
SEQ ID NO: 77, SEQ ID NO: 101, and SEQ ID NO: 129;
SEQ ID NO: 130, SEQ ID NO: 131, and SEQ ID NO: 132; and
SEQ ID NO: 115, SEQ ID NO: 133, and SEQ ID NO: 134; respectively.

In some embodiments, the present disclosure provides an anti-CDH17 antibody or an antigen-binding portion thereof, comprising at least one variable-heavy-chain-only single-domain or an antigen-binding portion thereof, wherein the at least one variable-heavy-chain-only (VHO) single-domain comprises an amino acid sequence with at least 85% identity to any one of SEQ ID NOs: 10-37 or an antigen-binding portion thereof.

In another aspect, the present disclosure provides an anti-CD3 antibody or an antigen-binding portion thereof. In some embodiments, the present disclosure provides an anti-CD3 antibody or an antigen-binding portion thereof comprising a heavy chain variable region comprising three Complementarity Determining Regions (CDRs), designated as HCDR1, HCDR2, and HCDR3, wherein the HCDR1, HCDR2, and HCDR3 are selected from:
SEQ ID NO: 135, SEQ ID NO: 136, and SEQ ID NO: 137;
SEQ ID NO: 135, SEQ ID NO: 138, and SEQ ID NO: 137; and
SEQ ID NO: 135, SEQ ID NO: 139, and SEQ ID NO: 137; respectively

In some embodiments, the present disclosure provides an anti-CD3 antibody or an antigen-binding portion thereof, comprising a light chain variable region comprising three CDRs, designated as LCDR1, LCDR2, and LCDR3, wherein the LCDR1, LCDR2, and LCDR3 are selected from:
SEQ ID NO: 140, SEQ ID NO: 141, and SEQ ID NO: 142; and
SEQ ID NO: 143, SEQ ID NO: 141, and SEQ ID NO: 142; respectively

In some embodiments, the disclosure provides an anti-CD3 antibody or an antigen binding fragment thereof comprising a heavy chain sequence comprising an amino acid sequence with at least 85% identity to any one of SEQ ID NOs: 39-41 or an antigen-binding portion thereof, and a light chain sequence comprising an amino acid sequence with at least 85% identity to any one of SEQ ID NOs: 43-44 or an antigen-binding portion thereof.

In some embodiments, the disclosure provides an anti-CD3 antibody or an antigen binding fragment thereof, comprising a heavy chain sequence and a light chain sequence comprising: SEQ ID NOs: 38 and 42; SEQ ID NOs: 38 and 43; SEQ ID NOs: 38 and 44; SEQ ID NOs: 39 and 42; SEQ ID NOs: 39 and 43; SEQ ID NOs: 39 and 44; SEQ ID NOs: 40 and 42; SEQ ID NOs: 40 and 43; SEQ ID NOs: 40 and 44; SEQ ID NOs: 41 and 42; SEQ ID NOs: 41 and 43; and SEQ ID NOs: 41 and 44; respectively.

The anti-CDH17 or anti-CD3 antibodies or antigen binding fragments thereof disclosed herein may be human, humanized, or chimeric antibodies, or antigen binding fragments.

The anti-CDH17 or anti-CD3 antibodies or antigen binding fragments thereof disclosed herein may be full length IgG1, IgG2, IgG3, or IgG4 antibodies or may be antigen-binding fragments thereof, such as a Fab, F(ab′)2, or scFv fragment. The antibody backbones may be modified to affect functionality, e.g., to eliminate residual effector functions.

In another aspect, the present disclosure provides a CD3×CDH17 bispecific antibody that binds to a protein linked to the CDH17 associated pathways and binds CD3 in T cells in the tumor microenvironment. For example, to prevent systemic activation of T cells, the CD3 and/or CDH17 moieties are masked to minimize T cell activation in normal tissues. By unmasking the CD3 and CDH17 in the tumor microenvironment, the bispecific antibody can activate the immune effector cell activity only at the targeted location. Therefore, the CD3×CDH17 bispecific antibody disclosed herein can take advantage of its added tumor cell growth inhibition efficacy in diseased tissues yet mitigating the increased toxicity concern by shielding the binding epitopes of the CD3 and CDH17 in normal tissues. There are a variety of different types of shielding to accommodate the different sequence families (e.g., light chain sequence families) for the CDH17 arm and the CD3 arm of the CD3×CDH17 bispecific antibody. The present disclosure provides a differentiated bispecific antibody with effective targeting of CDH17 for oncology indications, but with reduced safety concerns.

In one aspect, the present disclosure provides a bispecific antibody comprising:
a first binding arm comprising:
a first heavy chain fusion protein comprising, from the N- to the C-terminus, a mask A, a protease cleavable linker A, and an IgG heavy chain or an antigen-binding portion thereof, and
a first light chain fusion protein comprising, from the N- to the C-terminus, a mask B, a protease cleavable linker B, and an IgG light chain or an antigen-binding portion thereof,
wherein the IgG heavy chain or antigen-binding portion thereof and the IgG light chain or antigen-binding portion thereof of the first binding arm are capable of targeting a CD3 associated pathway and comprise an anti-CD3 antibody or an antigen binding fragment as described herein; and
a second binding arm comprising:
a second heavy chain fusion protein comprising, from the N- to the C-terminus, a mask C, a protease cleavable linker C, and an IgG heavy chain or an antigen-binding portion thereof; and
a second light chain fusion protein comprising, from the N- to the C-terminus, a mask D, a protease cleavable linker D, and an IgG light chain or an antigen-binding portion thereof,
wherein the IgG heavy chain or antigen-binding portion thereof and the IgG light chain or antigen-binding portion thereof of the second binding arm are capable of targeting a CDH17 associated pathway and comprise an anti-CDH17 antibody or an antigen binding fragment as described herein. The masks A-D can be the same or different from one another, and the protease cleavable linkers A-D can be the same or different from one another. The shields A-D and the protease sequences A-D are optional, meaning that some or all of them may be present or absent.

In another aspect, the present disclosure provides a bispecific antibody comprising:
a first binding arm comprising:
a first heavy chain fusion protein comprising, from the N- to the C-terminus, a mask A, a protease sequence A, and an IgG heavy chain or an antigen-binding portion thereof, and
a first light chain fusion protein comprising, from the N- to the C-terminus, a mask B, a protease sequence B, and an IgG light chain or an antigen-binding portion thereof,
wherein the IgG heavy chain or antigen-binding portion thereof and the IgG light chain or antigen-binding portion thereof of the first binding arm are capable of targeting a CD3 associated pathway and comprise an anti-CD3 antibody or an antigen binding fragment as described herein; and
a second binding arm comprising:
a second heavy chain fusion protein comprising, from the N- to the C-terminus, a mask C, a protease sequence C, and an IgG heavy chain comprising at least one variable-heavy-chain-only (VHO) single domain or an antigen-binding portion thereof,
wherein the at least one variable-heavy-chain-only single domain or antigen-binding portion thereof is capable of targeting a CDH17 associated pathway and comprises an anti-CDH17 VHO or an antigen binding fragment as described herein.
   The masks A-C can be the same or different from one another, and the protease sequences A-C can be the same or different from one another. The shields A-C and the protease sequences A-C are optional, meaning that some or all of them may be present or absent.

In certain embodiments, in a bispecific antibody disclosed herein,
the first binding arm comprises:
a first heavy chain fusion protein comprising, from the N- to the C-terminus, a signal sequence A-a mask A-a linker A-a protease cleavable linker A-a linker B-an IgG heavy chain or an antigen-binding portion thereof, and
a first light chain fusion protein comprising, from the N- to the C-terminus, a signal sequence B-a mask B-a linker C-a protease cleavable linker B-a linker D-an IgG light chain or an antigen-binding portion thereof,
wherein the IgG heavy chain or antigen-binding portion thereof and the IgG light chain or antigen-binding portion thereof of the first binding arm are capable of targeting a CD3 associated pathway and comprise an anti-CD3 antibody or an antigen binding fragment as described herein; and
the second binding arm comprises:
a second heavy chain fusion protein comprising, from the N- to the C-terminus, a signal sequence C-a mask C-a linker E-a protease cleavable linker C-a linker F-an IgG heavy chain or an antigen-binding portion thereof; and
a second light chain fusion protein comprising, from the N- to the C-terminus, a signal sequence D-a mask D-a linker G-a protease cleavable linker D-a linker H-an IgG light chain or an antigen-binding portion thereof,
wherein the IgG heavy chain or antigen-binding portion thereof and the IgG light chain or antigen-binding portion thereof of the second binding arm are capable of targeting a CDH17 associated pathway and comprise an anti-CDH17 antibody or an antigen binding fragment as described herein.
   The signal sequences A-D can be the same or different from one another, and the linkers A-H can be the same or different from one another. The signal sequences A-D, shields A-D, protease sequences A-D, and linkers A-H are optional, meaning that some or all of them may be present or absent.

In certain embodiments, the present disclosure provides a bispecific antibody comprising:
a first binding arm comprises:
a first heavy chain fusion protein comprising, from the N- to the C-terminus, a signal sequence A-a mask A-a linker A-a protease sequence A-a linker B-an IgG heavy chain or an antigen-binding portion thereof, and
a first light chain fusion protein comprising, from the N- to the C-terminus, a signal sequence B-a mask B-a linker C-a protease sequence B-a linker D-an IgG light chain or an antigen-binding portion thereof,
wherein the IgG heavy chain or antigen-binding portion thereof and the IgG light chain or antigen-binding portion thereof of the first binding arm are capable of targeting a CD3 associated pathway and comprise an anti-CD3 antibody or an antigen binding fragment as described herein; and
the second binding arm comprises:
a second heavy chain fusion protein comprising, from the N- to the C-terminus, a signal sequence C-a mask C-a linker E-a protease sequence C-a linker F-an IgG heavy chain comprising at least one variable-heavy-chain-only single-domain or an antigen-binding portion thereof,
wherein the at least one variable-heavy-chain-only single-domain or antigen-binding portion thereof is capable of targeting a CDH17 associated pathway and comprises an anti-CDH17 VHO or an antigen binding fragment as described herein.
   The signal sequences A-C can be the same or different from one another, and the linkers A-F can be the same or different from one another. The signal sequences A-C, shields A-C, protease sequences A-C, and linkers A-F are optional, meaning that some or all of them may be present or absent.

In certain embodiments, in a bispecific antibody disclosed herein, the IgG is human IgG1, IgG2, IgG3, or IgG4.

In certain embodiments, in a bispecific antibody disclosed herein, the first binding arm is monovalent, and the second binding arm is monovalent, bivalent, or multivalent.

In certain embodiments, in a bispecific antibody disclosed herein, the second binding arm comprises two or three IgG variable-heavy-chain-only single domains in tandem, wherein the two or three IgG variable-heavy-chain-only single domains are optionally connected via one or more linker sequences.

In certain embodiments, in a bispecific antibody disclosed herein, the first binding arm (anti-CD3 arm) comprises: a heavy chain variable region comprising three Complementarity Determining Regions (CDRs), designated as HCDR1, HCDR2, and HCDR3, wherein the HCDR1, HCDR2, and HCDR3 are selected from: SEQ ID NO: 135, SEQ ID NO: 136, and SEQ ID NO: 137; SEQ ID NO: 135, SEQ ID NO: 138, and SEQ ID NO: 137; and SEQ ID NO: 135, SEQ ID NO: 139, and SEQ ID NO: 137; respectively; and a light chain variable region comprising three CDRs, designated as LCDR1, LCDR2, and LCDR3, wherein the LCDR1, LCDR2, and LCDR3 are selected from: SEQ ID NO: 140, SEQ ID NO: 141, and SEQ ID NO: 142; and SEQ ID NO: 143, SEQ ID NO: 141, and SEQ ID NO: 142; respectively.

In certain embodiments, in a bispecific antibody disclosed herein, the second binding arm (anti-CDH17 arm) comprises: a heavy chain variable region comprising three Complementarity Determining Regions (CDRs), designated as HCDR1, HCDR2, and HCDR3, wherein the HCDR1, HCDR2, and HCDR3 are selected from: SEQ ID NO: 65, SEQ ID NO: 66, and SEQ ID NO: 67; and SEQ ID NO: 68, SEQ ID NO: 69, and SEQ ID NO: 70; respectively; and a light chain variable region comprising three CDRs, designated as LCDR1, LCDR2, and LCDR3, wherein the LCDR1, LCDR2, and LCDR3 are selected from: SEQ ID NO: 62, SEQ ID NO: 63, and SEQ ID NO: 64; and SEQ ID NO: 71, SEQ ID NO: 72, and SEQ ID NO: 73; respectively.

In certain embodiments, in a bispecific antibody disclosed herein, the second binding arm (anti-CDH17 arm) comprises at least one variable-heavy-chain-only single-domain or an antigen-binding portion thereof, wherein the at least one variable-heavy-chain-only single-domain comprises three CDRs (HCDR1, HCDR2, and HCDR3) selected from:
SEQ ID NO: 74, SEQ ID NO: 75, and SEQ ID NO: 76;
SEQ ID NO: 77, SEQ ID NO: 78, and SEQ ID NO: 79;
SEQ ID NO: 80, SEQ ID NO: 81, and SEQ ID NO: 82;
SEQ ID NO: 77, SEQ ID NO: 83, and SEQ ID NO: 84;
SEQ ID NO: 77, SEQ ID NO: 81, and SEQ ID NO: 85;
SEQ ID NO: 86, SEQ ID NO: 87, and SEQ ID NO: 88;
SEQ ID NO: 86, SEQ ID NO: 89, and SEQ ID NO: 90;
SEQ ID NO: 77, SEQ ID NO: 91, and SEQ ID NO: 92;
SEQ ID NO: 93, SEQ ID NO: 75, and SEQ ID NO: 94;
SEQ ID NO: 77, SEQ ID NO: 95, and SEQ ID NO: 96;
SEQ ID NO: 77, SEQ ID NO: 97, and SEQ ID NO: 98;
SEQ ID NO: 77, SEQ ID NO: 99, and SEQ ID NO: 100;
SEQ ID NO: 86, SEQ ID NO: 101, and SEQ ID NO: 102;
SEQ ID NO: 103, SEQ ID NO: 104, and SEQ ID NO: 105;
SEQ ID NO: 86, SEQ ID NO: 106, and SEQ ID NO: 107;
SEQ ID NO: 108, SEQ ID NO: 109, and SEQ ID NO: 110;
SEQ ID NO: 77, SEQ ID NO: 111, and SEQ ID NO: 112;
SEQ ID NO: 77, SEQ ID NO: 113, and SEQ ID NO: 114;
SEQ ID NO: 115, SEQ ID NO: 116, and SEQ ID NO: 117;
SEQ ID NO: 77, SEQ ID NO: 118, and SEQ ID NO: 119;
SEQ ID NO: 120, SEQ ID NO: 78, and SEQ ID NO: 121;
SEQ ID NO: 77, SEQ ID NO: 101, and SEQ ID NO: 122;
SEQ ID NO: 115, SEQ ID NO: 123, and SEQ ID NO: 124;
SEQ ID NO: 115, SEQ ID NO: 125, and SEQ ID NO: 126;
SEQ ID NO: 115, SEQ ID NO: 127, and SEQ ID NO: 128;
SEQ ID NO: 77, SEQ ID NO: 101, and SEQ ID NO: 129;
SEQ ID NO: 130, SEQ ID NO: 131, and SEQ ID NO: 132; and
SEQ ID NO: 115, SEQ ID NO: 133, and SEQ ID NO: 134; respectively.

In certain embodiments, a bispecific antibody disclosed herein is capable of binding a CD3 associated protein and a CDH17 associated protein simultaneously and is capable of inhibiting tumor cell proliferation.

In certain embodiments, in a bispecific antibody disclosed herein, the mask A, mask B, mask C, and mask D are each independently selected from the amino acid sequences set forth in SEQ ID NOs: 47-50. The anti-CD3 arm in a bispecific antibody disclosed herein can also utilize the mask as noted from the amino acid sequences set forth in SEQ ID NO: 51.

In certain embodiments, in a bispecific antibody disclosed herein, the protease cleavable linker A, protease cleavable linker B, protease cleavable linker C, and protease cleavable linker D are each independently selected from amino acid sequences set forth in SEQ ID NOs: 52-59.

In certain embodiments, in a bispecific antibody disclosed herein, the first binding arm (anti-CD3 arm) comprises a heavy chain amino acid sequence selected from SEQ ID NOs: 38-41, an amino acid sequence having at least 85% identity to any one of SEQ ID NOs: 38-41, or an antigen binding portion thereof; a light chain amino acid sequence selected from SEQ ID NOs: 42-43, an amino acid sequence having at least 85% identity to any one of SEQ ID NOs: 42-43, or an antigen binding portion thereof.

In certain embodiments, in a bispecific antibody disclosed herein, the second binding arm (anti-CDH17 arm) comprises a heavy chain amino acid sequence selected from SEQ ID NOs: 3, 4, 5, 7, and 8, an amino acid sequence having at least 85% identity to any one of SEQ ID NOs: 3, 4, 5, 7, and 8, or an antigen binding portion thereof; a light chain amino acid sequence selected from SEQ ID NOs: 2, 6, and 9, amino acid sequence having at least 85% identity to any one of SEQ ID NOs: 2, 6, and 9, or an antigen binding portion thereof.

In certain embodiments, in a bispecific antibody disclosed herein, the first binding arm (anti-CD3 arm) comprises a heavy chain sequence and a light chain amino acid sequence selected from SEQ ID NOs: 38 and 42; SEQ ID NOs: 38 and 43; SEQ ID NOs: 38 and 44; SEQ ID NOs: 39 and 42; SEQ ID NOs: 39 and 43 SEQ ID NOs: 39 and 44; SEQ ID NOs: 40 and 42; SEQ ID NOs: 40 and 43; SEQ ID NOs: 40 and 44; SEQ ID NOs: 41 and 42; SEQ ID NOs: 41 and 43; and SEQ ID NOs: 41 and 44; respectively.

In certain embodiments, in a bispecific antibody disclosed herein, the second binding arm (anti-CDH17 arm) comprises a heavy chain sequence and a light chain amino acid sequence selected from SEQ ID NO: 3 and 2; SEQ ID NO: 4 and 2; SEQ ID NO: 5 and 6; SEQ ID NO: 7 and 9; and SEQ ID NO: 8 and 9; respectively.

In certain embodiments, an antibody or bispecific antibody disclosed herein comprises a modified Fc to extend the half-life of the bispecific antibody, enhance resistance of the bispecific antibody to proteolytic degradation, reduce effector functionality of the bispecific antibody, facilitate generation of the bispecific antibody by Fc heterodimerization, facilitate multimerization of the bispecific antibody, and/or improve manufacturing and drug stability of the bispecific antibody.

In another aspect, the present disclosure provides a conjugate comprising an antibody or bispecific antibody disclosed herein conjugated to a moiety, such as a cytotoxic agent.

In another aspect, the present disclosure provides a composition comprising an antibody or bispecific antibody disclosed herein or a conjugate disclosed herein.

In another aspect, the present disclosure provides a pharmaceutical composition comprising an antibody or bispecific antibody disclosed herein or a conjugate disclosed herein, and a pharmaceutically acceptable carrier.

In another aspect, the present disclosure provides a nucleic acid encoding an anti-CDH17 antibody or an antigen-binding portion thereof.

In another aspect, the present disclosure provides a nucleic acid encoding an anti-CD3 antibody or an antigen-binding portion thereof.

In another aspect, the present disclosure provides a nucleic acid encoding a bispecific antibody, a first heavy chain fusion protein, a first light chain fusion protein, a second heavy chain fusion protein, or a second light chain fusion protein disclosed herein.

In another aspect, the present disclosure provides a recombinant vector, such as expression vector, comprising a nucleic acid disclosed herein.

In another aspect, the present disclosure provides a host cell comprising a recombinant vector such as an expression vector or a nucleic acid disclosed herein.

In another aspect, the present disclosure provides a method for preparing an antibody or bispecific antibody disclosed herein, comprising culturing a host cell disclosed herein, growing the host cell in a host cell culture, providing host cell culture conditions wherein a nucleic acid disclosed herein is expressed, and recovering the antibody or bispecific antibody from the host cell or from the host cell culture. In certain embodiments, the bispecific antibody is obtained using controlled Fab arm exchange.

In another aspect, the present disclosure provides a method for treating or preventing a CDH17 mediated disease or disorder in a subject in need thereof, comprising administering to the subject a pharmaceutically effective amount of an antibody, bispecific antibody, conjugate, or a pharmaceutical composition disclosed herein. In certain embodiments, the treatment or preventions reduces or prevents tumor growth and/or metastasis in the subject. In certain embodiments, the disease or disorder is lung cancer, breast cancer, colorectal cancer, or gastric cancer. In certain embodiments, the method for treating or preventing a disease or disorder, further comprises administering to the subject another therapeutic agent, before, during, or after administering the bispecific antibody or the pharmaceutical composition, wherein the subject suffers from relapsed CDH17 positive cancer.

In another aspect, the present disclosure provides a method for mediating CDH17 in a subject in need thereof, comprising administering to the subject an effective amount of an antibody or bispecific antibody or a pharmaceutical composition disclosed herein.

These and other embodiments of the present disclosure will be described in greater detail herein.

### BRIEF DESCRIPTION OF THE DRAWINGS

FIG. 1 shows mRNA expression profiles of CDH17.

FIG. 2 is a schematic diagram of the regulatory and signaling network of CDH17 in gastric cancer cells.

FIG. 3 shows that interactions between CDH17 and a2b1 integrin can promote cancer and a profile of how an antagonistic anti-CDH17 antibody can control cancer.

FIG. 4 is a schematic exhibition of a proposed signaling pathway modulated by CDH17 in gastric cancer cells.

FIG. 5 is a schematic drawing of a CDH17×CD3 bispecific antibody. FIG. 5A shows a shielded bispecific antibody that comprises two different sets of heavy chain (HC) and light chain (LC) pairing as indicated under the notations of “first arm” and “second arm.” Both arms can be shielded. FIG. 5B shows a bispecific antibody that comprises two different sets of heavy chain (HC) and light chain (LC) pairing without the shielding. FIG. 5C illustrates certain components of the CD3 arm (first arm) open reading frame and the CDH17 binding arm (second arm) open reading frame with the shielding. FIG. 5D illustrates certain components of the CD3 arm (first arm) open reading frame and the CDH17 binding arm (second arm) open reading frame without the shielding.

FIG. 6 is a schematic diagram of a CD3×CDH17 bispecific antibody comprising one or more anti-CDH17 variable-heavy-only (VHO) single domains. FIG. 6A shows two examples of shielded bispecific antibodies: a bispecific antibody that comprises a single VHO in the “second arm,” an arm targeting CDH17 (left panel); and a bispecific antibody that comprises a CDH17 binding arm (second arm) comprising two VHOs fused together (right panel). In both examples, the VHOs are connected with a masking domain. FIG. 6B shows two examples of non-shielded bispecific antibodies: a bispecific antibody that comprises a single VHO in the “second arm,” an arm targeting CDH17 (left panel); and a bispecific antibody that comprises a CDH17 binding arm (second arm) comprising two VHOs fused together. In both examples, the VHOs are not connected to a masking domain. FIG. 6C illustrates certain components of the CD3 arm (first arm) open reading frame and the CDH17 binding arm (second arm) open reading frame with the shielding. FIG. 6D illustrates certain components of the CD3 arm (first arm) open reading frame and the CDH17 binding arm (second arm) open reading frame without the shielding.

FIG. 7 is a schematic representation of protease digestion removal of the masking domains on a CDH17×CD3 bispecific antibody.

FIG. 8A shows reduced SDS-PAGE of CDH17 mAbs. CDH17 v1 represented a monoclonal antibody comprising a heavy chain variable region as set forth in SEQ ID NO: 3 and a light chain variable region as set forth in SEQ ID NO: 2; CDH17 v2 represented a monoclonal antibody comprising a heavy chain variable region as set forth in SEQ ID NO: 4 and a light chain variable region as set forth in SEQ ID NO: 2; CDH17 v3 represented a monoclonal antibody comprising a heavy chain variable region as set forth in SEQ ID NO: 5 and a light chain variable region as set forth in SEQ ID NO: 2; CDH17 v4 represented a monoclonal antibody comprising a heavy chain variable region as set forth in SEQ ID NO: 3 and a light chain variable region as set forth in SEQ ID NO: 6; CDH17 v5 represented a monoclonal antibody comprising a heavy chain variable region as set forth in SEQ ID NO: 4 and a light chain variable region as set forth in SEQ ID NO: 6; and CDH17 v6 represented a monoclonal antibody comprising a heavy chain variable region as set forth in SEQ ID NO: 5 and a light chain variable region as set forth in SEQ ID NO: 6. FIG. 8B shows size exclusion chromatography on a HPLC of CDH17 v1 showing the typical SEC profile of a monodisperse and monomeric human IgG1 monoclonal antibody. FIG. 8C shows size-exclusion chromatography on a HPLC of CDH17 v2 showing the typical SEC profile of a monodisperse and monomeric human IgG1 monoclonal antibody. The SEC chromatograms have y axes for absorbance at 280 nm and the x axes for HPLC retention time in minutes.

FIG. 9 shows SEC characterizations of CD3×CDH17 bispecific antibodies. FIG. 9A shows size exclusion chromatography (SEC) on a HPLC of SP34×CDH17 v1. The SP34 component is based on a monoclonal antibody comprising a heavy chain variable region as set forth in SEQ ID NO: 45 and a light chain variable region as set forth in SEQ ID NO: 46. The CDH17 v1 component is based on a monoclonal antibody comprising a heavy chain variable region as set forth in SEQ ID NO: 3 and a light chain variable region as set forth in SEQ ID NO: 2. The SEC shows that the bispecific antibody represents 97.2% monomer. FIG. 9B shows size exclusion chromatography (SEC) on a HPLC of SP34×CDH17 v2. The SP34 component is based on a monoclonal antibody comprising a heavy chain variable region as set forth in SEQ ID NO: 45 and a light chain variable region as set forth in SEQ ID NO: 46. The CDH17 v2 component is based on a monoclonal antibody comprising a heavy chain variable region as set forth in SEQ ID NO: 4 and a light chain variable region as set forth in SEQ ID NO: 2. The SEC shows that the bispecific antibody represents 97.7% monomer. The chromatograms have y axes for absorbance at 220 nm and the x axes for retention time in minutes.

FIG. 10 demonstrated concentration dependent ELISA binding of anti-CD3 antibodies to recombinant human CD3 delta and epsilon domain protein in an ELISA assay. The y axis is the binding response expressed in optical density at 450 nm absorbance units. The x axis is the concentration of the molecules tested. SP34 had potent binding to human CD3 delta and epsilon domain protein

FIG. 11A shows the binding of anti-CDH17 mAbs to recombinant human CDH17 in ELISA assays. CDH17 v1, CDH17 v2, CDH17 v3, CDH17 v4, CDH17 v5, and CDH17 v6 all have binding to CDH17. FIG. 11B shows the binding of anti-CDH17 v1 and CDH17 v2 mAbs and VHO v366, VHO v364, VHO v363, and VHO v358 to recombinant human CDH17 in ELISA assays. VHO v366 represented a monoclonal antibody comprising a heavy chain variable region as set forth in SEQ ID NO: 13. VHO v364 represented a monoclonal antibody comprising a heavy chain variable region as set forth in SEQ ID NO: 14. VHO v363 represented a monoclonal antibody comprising a heavy chain variable region as set forth in SEQ ID NO: 15. VHO v358 represented a monoclonal antibody comprising a heavy chain variable region as set forth in SEQ ID NO: 19. These aforementioned single domain antibodies bound to CDH17 via ELISA assay.

FIG. 12 demonstrated that the anti-CDH17 single domain Abs bound to (FIG. 12A) CDH17 expressing AGS cells and (FIG. 12B) CDH17 expressing AsPC-1 pancreatic cancer cells by flow cytometry. VHO v349 represented a monoclonal antibody comprising heavy chain variable regions as set forth in SEQ ID NO: 10. VHO v369 represented a monoclonal antibody comprising a heavy chain variable region as set forth in SEQ ID NO: 11. VHO v364 represented a monoclonal antibody comprising a heavy chain variable region as set forth in SEQ ID NO: 14. VHO v347 represented a monoclonal antibody comprising a heavy chain variable region as set forth in SEQ ID NO: 12. VHO v366 represented a monoclonal antibody comprising a heavy chain variable region as set forth in SEQ ID NO: 13. VHO v341 represented a monoclonal antibody comprising a heavy chain variable region as set forth in SEQ ID NO: 16. VHO v370 represented a monoclonal antibody comprising a heavy chain variable region as set forth in SEQ ID NO: 33. VHO v343 represented a monoclonal antibody comprising a heavy chain variable region as set forth in SEQ ID NO: 27. VHO v363 represented a monoclonal antibody comprising a heavy chain variable region as set forth in SEQ ID NO: 15. CDH17-VHO364 was used as a reference binder for these CDH17 ELISA binding experiments.

FIG. 13 demonstrated that the anti-CDH17 single domain Abs bound to (FIG. 13A) CDH17 expressing AGS cells and (FIG. 13B) CDH17 expressing AsPC-1 pancreatic cancer cells by flow cytometry. VHO v352 represented a monoclonal antibody comprising a heavy chain variable region as set forth in SEQ ID NO: 18. VHO v357 represented a monoclonal antibody comprising a heavy chain variable region as set forth in SEQ ID NO: 17. VHO v362 represented a monoclonal antibody comprising a heavy chain variable region as set forth in SEQ ID NO: 20. VHO v351 represented a monoclonal antibody comprising a heavy chain variable region as set forth in SEQ ID NO: 30. VHO v358 represented a monoclonal antibody comprising a heavy chain variable region as set forth in SEQ ID NO: 19. VHO v342 represented a monoclonal antibody comprising a heavy chain variable region as set forth in SEQ ID NO: 26. VHO v345 represented a monoclonal antibody comprising a heavy chain variable region as set forth in SEQ ID NO: 28. VHO v355 represented a monoclonal antibody comprising a heavy chain variable region as set forth in SEQ ID NO: 31. VHO v346 represented a monoclonal antibody comprising a heavy chain variable region as set forth in SEQ ID NO: 29. CDH17-VHO364 was used as a reference binder for these CDH17 ELISA binding experiments.

FIG. 14 demonstrated that the anti-CDH17 single domain Abs bound to (FIG. 14A) CDH17 expressing AGS cells and (FIG. 14B) CDH17 expressing AsPC-1 pancreatic cancer cells by flow cytometry. VHO v365 represented a monoclonal antibody comprising a heavy chain variable region as set forth in SEQ ID NO: 21. VHO v376 represented a monoclonal antibody comprising a heavy chain variable region as set forth in SEQ ID NO: 145. VHO v372 represented a monoclonal antibody comprising a heavy chain variable region as set forth in SEQ ID NO: 23. VHO v374 represented a monoclonal antibody comprising a heavy chain variable region as set forth in SEQ ID NO: 24. VHO v378 represented a monoclonal antibody comprising a heavy chain variable region as set forth in SEQ ID NO: 36. VHO v379 represented a monoclonal antibody comprising a heavy chain variable region as set forth in SEQ ID NO: 25. VHO v380 represented a monoclonal antibody comprising a heavy chain variable region as set forth in SEQ ID NO: 37. VHO v371 represented a monoclonal antibody comprising a heavy chain variable region as set forth in SEQ ID NO: 34. VHO v377 represented a monoclonal antibody comprising a heavy chain variable region as set forth in SEQ ID NO: 35. CDH17-VHO364 was used as a reference binder for these CDH17 ELISA binding experiments.

FIG. 15 demonstrated that the tandem anti-CDH17 single domain Abs bound to CDH17 expressing AsPC-1 pancreatic cancer cells by flow cytometry. The tandem anti-CDH17 single domain antibody CDH17_v364_376 was a human IgG1 Fc antibody fusion comprising an amino-terminal anti-CDH17 heavy chain VHO v364 (SEQ ID NO: 14) fused to VHO v376 (SEQ ID NO: 145) via a (GGGGS)₄ linker on a human IgG1 Fc. CDH17_v349_364 was a human IgG1 Fc antibody fusion comprising an amino-terminal anti-CDH17 heavy chain VHO v349 (SEQ ID NO: 10) fused to VHO v364 (SEQ ID NO: 14) via a (GGGGS)₄ (SEQ ID NO: 144) linker on a human IgG1 Fc. CDH17_v376_364 was a human IgG1 Fc antibody fusion comprising an amino-terminal anti-CDH17 heavy chain VHO v376 fused to VHO v364 via a (GGGGS)₄ linker on a human IgG1 Fc. CDH17_v376_349 was a human IgG1 Fc antibody fusion comprising an amino-terminal anti-CDH17 heavy chain VHO v376 fused to VHO v349 via a (GGGGS)₄ linker on a human IgG1 Fc. CDH17_v349_376 was a human IgG1 Fc antibody fusion comprising an amino-terminal anti-CDH17 heavy chain VHO v349 fused to VHO v376 via a (GGGGS)₄ linker on a human IgG1 Fc.

FIG. 16 demonstrated dose responses of CDH17×CD3 bispecific antibody PBMC T cell activation in the presence of the CDH17 bearing AsPC-1 pancreatic cancer cells. The CD3 arm was based on the SP34 antibody comprising variable heavy chain SEQ ID NO: 45 and variable light chain SEQ ID NO: 46. The CDH17_v1 was based on an antibody comprising variable heavy chain SEQ ID NO: 38 and variable light chain SEQ ID NO: 42. The CDH17_v2 was based on an antibody comprising variable heavy chain SEQ ID NO: 39 and variable light chain SEQ ID NO: 43. FIG. 16A: CD4+ T cell activation with AsPC-1 cells with the y axis being the percent of CD25+ CD69+ T cells. FIG. 16B: CD4+ T cell activation with AsPC-1 cells with the y axis being the percent of CD25− CD69+ T cells. FIG. 16C: CD8+ T cell activation with AsPC-1 cells with the y axis being the percent of CD25+ CD69+ T cells. FIG. 16D: CD4+ T cell activation with AsPC-1 cells with the y axis being the percent of CD25− CD69+ T cells. The x axes for FIG. 16A-D were the concentration of the molecules tested.

FIG. 17 demonstrated dose responses of CDH17×CD3 bispecific antibody directed primary human T cell killing of AsPC-1, a CDH17 bearing pancreatic cancer cell line. The CD3 arm was based on the SP34 antibody comprising variable heavy chain SEQ ID NO: 45 and variable light chain SEQ ID NO: 46. The CDH17_v1 was based on an antibody comprising variable heavy chain SEQ ID NO: 3 and variable light chain SEQ ID NO: 2. The CDH17_v2 was based on an antibody comprising variable heavy chain SEQ ID NO: 4 and variable light chain SEQ ID NO: 2. Potent T cell killing responses were observed. The y axis is the percent cell lysis. The x axis is the concentration of the molecules assessed.

### DETAILED DESCRIPTION OF THE DISCLOSURE

### Definitions

All publications, including but not limited to disclosures and disclosure applications, cited in this specification are herein incorporated by reference as though fully set forth. If certain content of a reference cited herein contradicts or is inconsistent with the present disclosure, the present disclosure controls.

Any one embodiment of the disclosure described herein, including those described only in one section of the specification describing a specific aspect of the disclosure, and those described only in the examples or drawings, can be combined with any other one or more embodiment(s), unless explicitly disclaimed or improper.

It is to be understood that the terminology used herein is for the purpose of describing particular embodiments only and is not intended to be limiting. Unless defined otherwise, all technical and scientific terms used herein have the same meaning as commonly understood by one of ordinary skill in the art to which the disclosure pertains.

Although any methods and materials similar or equivalent to those described herein may be used in the practice for testing of the present disclosure, exemplary materials and methods are described herein. In describing and claiming the present disclosure, the following terminology will be used.

As used in this specification and the appended claims, the singular forms “a,” “an,” and “the” include plural referents unless the content clearly dictates otherwise. Thus, for example, reference to “a cell” includes a combination of two or more cells, and the like.

“Antibodies” is meant in a broad sense and includes immunoglobulin molecules including monoclonal antibodies including murine, human, humanized and chimeric monoclonal antibodies, antibody fragments, bispecific or multi-specific antibodies, dimeric, tetrameric or multimeric antibodies, single chain antibodies, domain antibodies and any other modified configuration of the immunoglobulin molecule that comprises an antigen binding site of the required specificity.

“Full length antibody molecules” are comprised of two heavy chains (HC) and two light chains (LC) inter-connected by disulfide bonds as well as multimers thereof (e.g., IgM). Each heavy chain is comprised of a heavy chain variable region (V_{H}) and a heavy chain constant region (comprised of domains C_{H1}, hinge, C_{H2} and C_{H3}). Each light chain is comprised of a light chain variable region (V_{L}) and a light chain constant region (C_{L}). The V_{H} and the V_{L} regions may be further subdivided into regions of hyper variability, termed complementarity determining regions (CDR), interspersed with framework regions (FR). Each V_{H} and V_{L} is composed of three CDRs and four FR segments, arranged from amino-to-carboxyl-terminus in the following order: FR1, CDR1, FR2, CDR2, FR3, CDR3 and FR4.

“Complementarity determining regions (CDR)” are “antigen binding sites” in an antibody. CDRs may be defined using various terms: (i) Complementarity Determining Regions (CDRs), three in the V_{H} (HCDR1, HCDR2, HCDR3) and three in the V_{L} (LCDR1, LCDR2, LCDR3) are based on sequence variability (Wu and Kabat 1970) (Kabat et al., Sequences of Proteins of Immunological Interest, 5th Ed. Public Health Service, National Institutes of Health, Bethesda, Md., 1991). (ii) “Hypervariable regions,” “HVR,” or “HV,” three in the V_{H} (H1, H2, H3) and three in the V_{L} (L1, L2, L3) refer to the regions of an antibody variable domains which are hypervariable in structure as defined by Chothia and Lesk (Chothia and Lesk 1987). The International ImMunoGeneTics (IMGT) database (http://www_imgt_org) provides a standardized numbering and definition of antigen-binding sites. The correspondence between CDRs, HVs, and IMGT delineations are described (Lefranc, Pommie et al. 2003). The term “CDR,” “HCDR1,” “HCDR2,” “HCDR3,” “LCDR1,” “LCDR2” and “LCDR3” as used herein includes CDRs defined by any of the methods described supra, Kabat, Chothia or IMGT, unless otherwise explicitly stated in the specification.

Immunoglobulins may be assigned to five major classes, IgA, IgD, IgE, IgG and IgM, depending on the heavy chain constant region amino acid sequence. IgA and IgG are further sub-classified as the isotypes IgA₁, IgA₂, IgG₁, IgG₂, IgG₃ and IgG₄. Antibody light chains of any vertebrate species may be assigned to one of two clearly distinct types, namely kappa (κ) and lambda (λ), based on the amino acid sequences of their constant regions.

“Antibody fragment,” “antigen binding fragment,” or “antigen binding portion,” refers to a portion of an immunoglobulin molecule that retains the heavy chain and/or the light chain antigen binding site, such as heavy chain complementarity determining regions (HCDR) 1, 2 and 3, light chain complementarity determining regions (LCDR) 1, 2 and 3, a heavy chain variable region (V_{H}), or a light chain variable region (V_{L}). Antibody fragments include well known F_{ab}, F(_{ab′})2, F_{d} and Fᵥ fragments as well as domain antibodies (dAb) consisting of one V_{H} domain. V_{H} and V_{L} domains may be linked together via a synthetic linker to form various types of single chain antibody designs where the V_{H}/V_{L} domains may pair intramolecularly, or intermolecularly in those cases when the V_{H} and V_{L} domains are expressed by separate single chain antibody constructs, to form a monovalent antigen binding site, such as single chain Fv (scFv) or diabody; described for example in Int. Patent Publ. Nos. WO1998/44001, WO1988/01649, WO1994/13804, and WO1992/01047.

“Antibody mimetic” refers to an engineered antibody protein that exhibits specific binding to a target. For example, antibody mimetic can be an Affibody, a DARPin, an Anticalin, an Avimer, a Versa body, or a Duocalin.

“Monoclonal antibody” refers to an antibody population with single amino acid composition in each heavy and each light chain, except for possible well-known alterations such as removal of C-terminal lysine from the antibody heavy chain. Monoclonal antibodies typically bind one antigenic epitope, except that, e.g., bispecific monoclonal antibodies bind to two distinct antigenic epitopes. Monoclonal antibodies may have heterogeneous glycosylation within the antibody population. Monoclonal antibody may be monospecific or multi-specific, or monovalent, bivalent or multivalent. A bispecific antibody is included in the term monoclonal antibody.

“Isolated antibody” refers to an antibody or antibody fragment that is substantially free of other antibodies having different antigenic specificities. “Isolated antibody” encompasses antibodies that are isolated to a higher purity, such as antibodies that are at least 80%, such as 80%, 81%, 82%, 83%, 84%, 85%, 86%, 87%, 88%, 89%, 90%, 91%, 92%, 93%, 94%, 95%, 96%, 97%, 98%, 99%, or 100% pure.

“Humanized antibody” refers to an antibody in which the antigen binding sites are derived from non-human species and the variable region frameworks are derived from human immunoglobulin sequences. Humanized antibody may include substitutions in the framework so that the framework may not be an exact copy of expressed human immunoglobulin or human immunoglobulin germline gene sequences.

“Human antibody” refers to an antibody having heavy and light chain variable regions in which both the framework and the antigen binding site are derived from sequences of human origin and is optimized to have minimal immune response when administered to a human subject. If the antibody contains a constant region or a portion of the constant region, the constant region also is derived from sequences of human origin.

“Anti-target” refers to an antibody or antibody domain (also referred to as an antigen-binding portion or fragment of an antibody) that can bind to the specified target molecule such as CDH17 (i.e., anti-CDH17 is an antibody or antibody domain that can bind to CDH17). The style “CDH17” refers to the CDH17 protein or CDH17 gene product. The style “CDH17” refers to the CDH17 gene.

“CD3×CDH17” refers to a bispecific antibody or antibody fragments that can bind to CD3 and CDH17. The process of making bispecific antibodies requires recombinant modifications to either of parental mAb amino acid sequences. Although the amino acid sequences of the CH1, CL, and Fc domains of each parental mAb may not be the same, there is no significant difference in the binding between the CD3×CDH17 and CDH17×CD3 bispecific antibodies. The notations of “first arm” (or “first binding arm”) and “second arm” (or “second binding arm”) herein are arbitrary. For example, in a bispecific antibody disclosed herein, a first arm could target CD3, and a second arm could target CDH17; or a first arm could target CDH17, and a second arm could target CD3.

The numbering of amino acid residues in the antibody constant region throughout the specification is according to the EU index as described in Kabat et al., Sequences of Proteins of Immunological Interest, 5th Ed. Public Health Service, National Institutes of Health, Bethesda, MD. (1991), unless otherwise explicitly stated.

Conventional one and three-letter amino acid codes are used herein as shown in Table 1.

TABLE 1

| | Three-letter | |
|---|---|---|
| Amino acid | code | One-letter code |
| Alanine | Ala | A |
| Arginine | Arg | R |
| Asparagine | Asn | N |
| Aspartate | Asp | D |
| Cysteine | Cys | C |
| Glutamate | Gln | E |
| Glutamine | Glu | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

The polypeptides, nucleic acids, fusion proteins, and other compositions provided herein may encompass polypeptides, nucleic acids, fusion proteins, and the like that have a recited percent identity to an amino acid sequence or DNA sequence provided herein. The term “identity” refers to a relationship between the sequences of two or more polypeptide molecules or two or more nucleic acid molecules, as determined by aligning and comparing the sequences. “Percent identity,” “percent homology,” “sequence identity,” or “sequence homology” and the like mean the percent of identical residues between the amino acids or nucleotides in the compared molecules and is calculated based on the size of the smallest of the molecules being compared. For example, a sequence A that is “at least 85% identity” to a sequence B means that sequence A comprises at least 85%, e.g., at least 86%, 87%, 88%, 89%, 90%, 91%, 92%, 93%, 94%, 95%, 96%, 97%, 98%, or 99%, identical residues to those of sequence B. For these calculations, gaps in alignments (if any) are preferably addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al., 1988, SIAM J. Applied Math. 48:1073. In calculating percent identity, the sequences being compared are typically aligned in a way that gives the largest match between the sequences.

The constant region sequences of the mammalian IgG heavy chain are designated in sequence as C_{H1}-hinge-C_{H2}-C_{H3}. The “hinge,” “hinge region” or “hinge domain” of an IgG is generally defined as including Glu216 and terminating at Pro230 of human IgG₁ according to the EU Index but functionally, the flexible portion of the chain may be considered to include additional residues termed the upper and lower hinge regions, such as from Glu216 to Gly237 and the lower hinge has been referred to as residues 233 to 239 of the F_{c} region where F_{c}γR binding was generally attributed. Hinge regions of other IgG isotypes may be aligned with the IgG₁ sequence by placing the first and last cysteine residues forming inter-heavy chain S—S bonds. Although boundaries may vary slightly, as numbered according to the EU Index, the C_{H1} domain is adjacent to the V_{H} domain and amino terminal to the hinge region of an immunoglobulin heavy chain molecule and includes the first (most amino terminal) constant region of an immunoglobulin heavy chain, e.g., from about EU positions 118-215. The F_{c} domain extends from amino acid 231 to amino acid 447; the C_{H2} domain is from about Ala231 to Lys340 or Gly341 and the C_{H3} from about Gly341 or Gln342 to Lys447. The residues of the IgG heavy chain constant region of the C_{H1} region terminate at Lys. The F_{c} domain containing molecule comprises at least the C_{H2} and the C_{H3} domains of an antibody constant region, and therefore comprises at least a region from about Ala231 to Lys447 of IgG heavy chain constant region. The F_{c} domain containing molecule may optionally comprise at least portion of the hinge region.

“Epitope” refers to a portion of an antigen (e.g., CD3 or CDH17) to which an antibody specifically binds. Epitopes typically consist of chemically active (such as polar, non-polar, or hydrophobic) surface groupings of moieties such as amino acids or polysaccharide side chains and may have specific three-dimensional structural characteristics, as well as specific charge characteristics. An epitope may be composed of contiguous and/or discontiguous amino acids that form a conformational spatial unit. For a discontiguous epitope, amino acids from differing portions of the linear sequence of the antigen come in close proximity in 3-dimensional space through the folding of the protein molecule. Antibody “epitope” depends on the methodology used to identify the epitope.

A “leader sequence” (also referred to as “signal peptide” or “signal sequence”) as used herein includes any signal peptide that can be processed by a mammalian cell, including the human B2M leader. Such sequences are well-known in the art.

A “cleavable linker” (also referred to as “protease sequence” or “protease sequence cleavable linker”) is a peptide substrate cleavable by an enzyme. Operatively, the cleavable linker, upon being cleaved by the enzyme, allows for activation of the present shielded antibody with a masking domain. Preferably, the cleavable linker is selected so that activation occurs at the desired site of action, which can be a site in or near the target cells (e.g., carcinoma cells) or tissues. For example, the cleavable linker is a peptide substrate specific for an enzyme that is specifically or highly expressed in the site of action, such that the cleavage rate of the cleavable linker in the target site is greater than that in sites other than the target site.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. The terms also include polypeptides that have co-translational (e.g., signal peptide cleavage) and post-translational modifications of the polypeptide, such as, for example, disulfide-bond formation, glycosylation, acetylation, phosphorylation, proteolytic cleavage, and the like.

Furthermore, as used herein, a “polypeptide” refers to a protein that includes modifications, such as deletions, additions, and substitutions (generally conservative in nature as would be known to a person in the art) to the native sequence, as long as the protein maintains the desired activity. These modifications can be deliberate, as through site-directed mutagenesis, or can be accidental, such as through mutations of hosts that produce the proteins, or errors due to PCR amplification or other recombinant DNA methods.

The term “masking domain” (also referred to as “shield,” “shielding domain,” or “mask”) in this disclosure refers to a protein domain that can be fused to an antibody and mask the antibody in binding to its antigen. The shielding domain can mask the antibody from recognizing its target epitope, so the antibody is kept as an inactive shielded antibody form. Upon the removal of the shielding domain, the variable domains of the antibody are exposed and can bind and exert actions to its target.

The term “recombinant,” as used herein to describe a nucleic acid molecule, means a polynucleotide of genomic, cDNA, viral, semisynthetic, and/or synthetic origin, which, by virtue of its origin or manipulation, is not associated with all or a portion of the polynucleotide sequences with which it is associated in nature. The term “recombinant,” as used with respect to a protein or polypeptide, refers to a polypeptide produced by expression from a recombinant polynucleotide. The term “recombinant,” as used with respect to a host cell or a virus, refers to a host cell or virus into which a recombinant polynucleotide has been introduced. Recombinant is also used herein with reference to a material (e.g., a cell, a nucleic acid, a protein, or a vector) that the material has been modified by the introduction of a heterologous material (e.g., a cell, a nucleic acid, a protein, or a vector).

The terms “polynucleotide,” “oligonucleotide,” “nucleic acid” and “nucleic acid molecule” are used interchangeably herein to include a polymeric form of nucleotides, either ribonucleotides or deoxyribonucleotides. This term refers only to the primary structure of the molecule.

“Vector” refers to a polynucleotide capable of being duplicated within a biological system or that can be moved between such systems. Vector polynucleotides typically contain elements, such as origins of replication, polyadenylation signal or selection markers, that function to facilitate the duplication or maintenance of these polynucleotides in a biological system, such as a cell, virus, animal, plant, and reconstituted biological systems utilizing biological components capable of duplicating a vector. The vector polynucleotide may be DNA or RNA molecules, cDNA, or a hybrid of these, single stranded or double stranded.

“Expression vector” refers to a vector that can be utilized in a biological system or in a reconstituted biological system to direct the translation of a polypeptide encoded by a polynucleotide sequence present in the expression vector.

As used herein, the term “heterologous” used in reference to nucleic acid sequences, proteins or polypeptides, means that these molecules are not naturally occurring in the cell from which the heterologous nucleic acid sequence, protein or polypeptide was derived. For example, the nucleic acid sequence coding for a human polypeptide that is inserted into a cell that is not a human cell is a heterologous nucleic acid sequence in that particular context. Whereas heterologous nucleic acids may be derived from different organism or animal species, such nucleic acid need not be derived from separate organism species to be heterologous. For example, in some instances, a synthetic nucleic acid sequence or a polypeptide encoded therefrom may be heterologous to a cell into which it is introduced in that the cell did not previously contain the synthetic nucleic acid. As such, a synthetic nucleic acid sequence or a polypeptide encoded therefrom may be considered heterologous to a human cell, e.g., even if one or more components of the synthetic nucleic acid sequence or a polypeptide encoded therefrom was originally derived from a human cell.

A “host cell,” as used herein, denotes a cell of any type that is capable of being transformed with a nucleic acid or vector of the disclosure so as to produce a polypeptide encoded thereby. For example, the host cell can be an in vivo or in vitro eukaryotic cell or a cell from a multicellular organism (e.g., a cell line) cultured as a unicellular entity, which eukaryotic cells can be, or have been, used as recipients for a nucleic acid (e.g., an expression vector that comprises a nucleotide sequence encoding a multimeric polypeptide of the present disclosure), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector. For example, a genetically modified eukaryotic host cell is genetically modified by virtue of introduction into a suitable eukaryotic host cell a heterologous nucleic acid, e.g., an exogenous nucleic acid that is foreign to the eukaryotic host cell, or a recombinant nucleic acid that is not normally found in the eukaryotic host cell.

“Specific binding” or “specifically binds” or “binds” refer to an antibody binding to a specific antigen with greater affinity than for other antigens. Typically, the antibody “specifically binds” when the equilibrium dissociation constant (K_{D}) for binding is about 1×10⁻⁸ M or less, for example about 1×10⁻⁹ M or less, about 1×10⁻¹⁰ M or less, about 1×10⁻¹¹ M or less, or about 1×10⁻¹² M or less, typically with the K_{D} that is at least one hundred-fold less than its K_{D} for binding to a non-specific antigen (e.g., BSA, casein). The K_{D} may be measured using standard procedures.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, murines (e.g., rats, mice), lagomorphs (e.g., rabbits), non-human primates, humans, canines, felines, ungulates (e.g., equines, bovines, ovines, porcines, caprines), etc.

A “therapeutically effective amount,” “pharmaceutically effective amount,” “effective amount,” or “efficacious amount” refers to the amount of an agent, or combined amounts of two agents, that, when administered to a mammal or other subject for treating a disease, is sufficient to affect such treatment for the disease. The “therapeutically effective amount” will vary depending on the agent(s), the disease and its severity and the age, weight, etc., of the subject to be treated.

Before the present disclosure is further described, it is to be understood that this disclosure is not limited to particular embodiments described, as such may, of course, vary. It is also to be understood that the terminology used herein is for the purpose of describing particular embodiments only and is not intended to be limiting.

The following examples described the disclosure in further details which are not intended to limit the scope of protection for the disclosure.

### CDH17 and Anti-CDH17 Antibodies

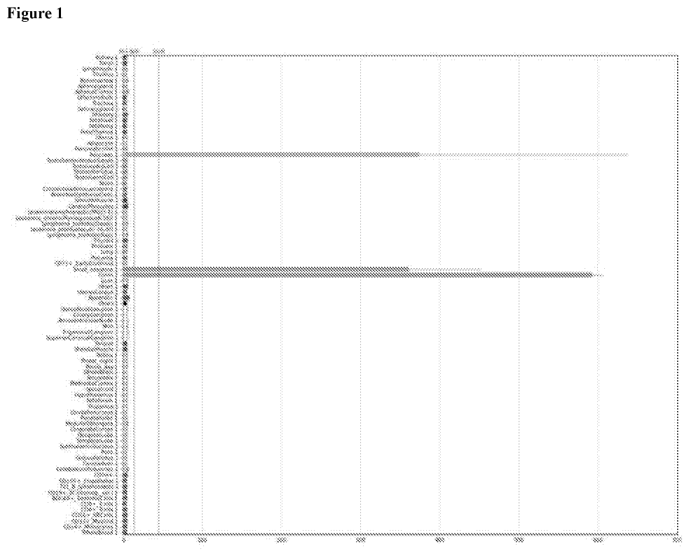

FIG. 1 shows mRNA expression profiles of CDH17. As shown in FIG. 1, CDH17 expression is higher in pancreas, small intestine, and colon tissues. This figure is generated using the BioGPS software (Su, Wiltshire et al. 2004, Wu, Jin et al. 2016).

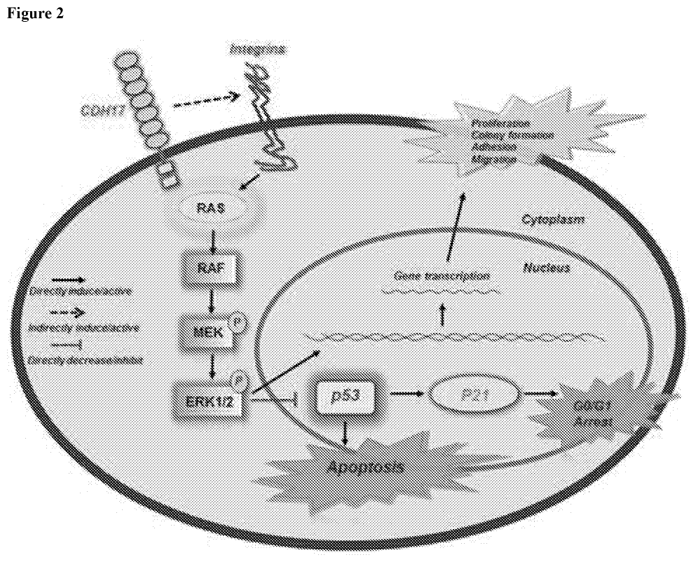

FIG. 2 is a schematic diagram of the regulatory and signaling network of CDH17 in GC. This schematic diagram demonstrates the inducing effect of CDH17 on the Ras/Raf/MEK/ERK signaling pathway and illustrates the hypothetic involvement of integrins in Gastric cancer (Lin, Zhang et al. 2014). CDH17 indirectly affects integrins to stabilize their structure and activity. The up-regulation of cadherin-integrin signaling activates the Ras/Raf/MEK/ERK pathway. The activation of ERK regulates various nuclear and cytoplasmic substrates, including p53 and p21, which involve in diverse cellular responses, such as cell proliferation, migration, adhesion, colony formation, cell-cycle, and apoptosis.

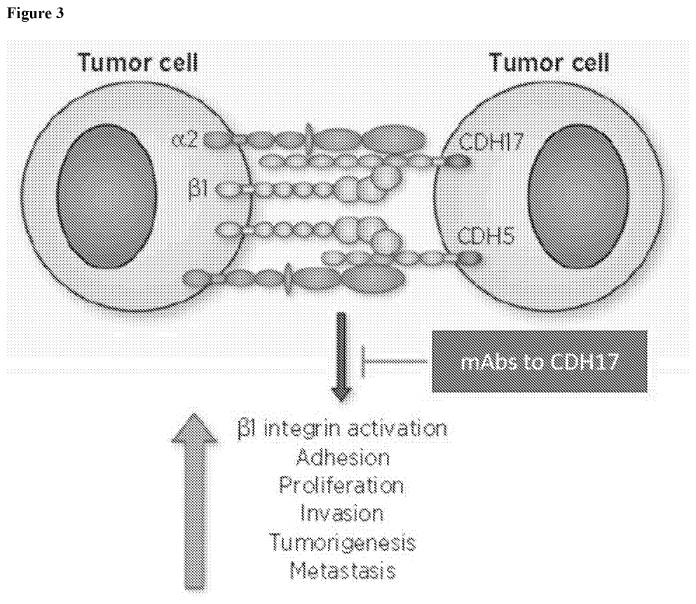

As shown in FIG. 3, interactions between CDH17 and a2ß1 integrin can promote cancer. The RGD-containing domain of CDH17 or CDH5 (also known as VE-cadherin) is bound by integrin a2ß1 on an adjacent tumor cell (adapted from Marshall et al. (Marshall 2018). This results in enhanced integrin activation, adhesion, proliferation, invasion, tumorigenesis, and metastasis.

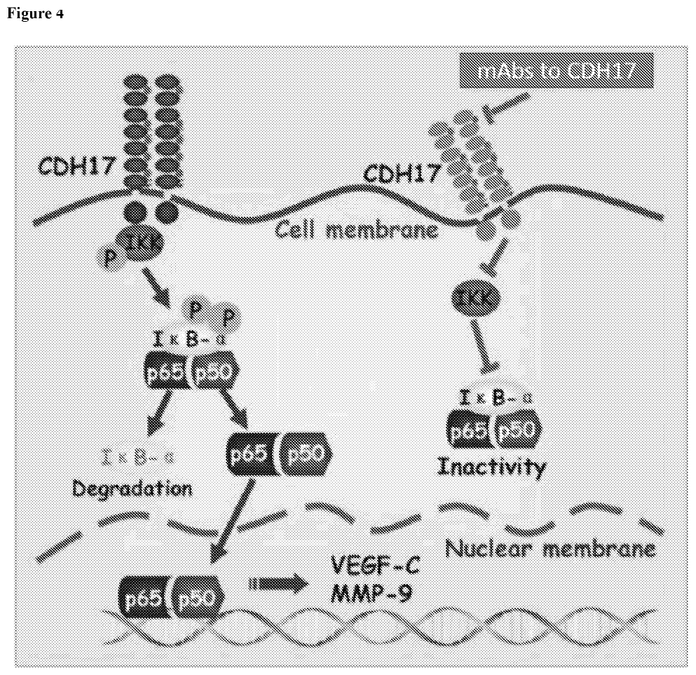

FIG. 4 is a schematic exhibition of a proposed signaling pathway modulated by CDH17 in gastric cancer cells. Coupling CDH17 activates the IKK complex, which subsequently phosphorylates the IκB-α (adapted from Li et al., 2017 (Li, Yang et al. 2017). Then the phosphorylated IκB-α undergoes proteasome-dependent decomposition, which releases the heterodimers of p65/p50 into cytoplasm to be transferred into the nucleus. Finally, the p65 binds to its responsive gene and promotes the transcription of downstream proteins including MMP-9. On the other hand, inhibition of CDH17 can attenuate the activation of NFκB in gastric cancer cells, leading to a concomitant reduction in downstream proteins.

The primary amino acid sequence of human Cadherin-17 is set forth in SEQ NO: 1 in Table 2.

TABLE 2

| SEQ | | |
|---|---|---|
| ID | | |
| NO: | Description | Amino Acid Sequence |
| 1 | Human | MILQAHLHSLCLLMLYLATGYGQEGKFSGPLKPMTFSIYEGQEPSQIIFQFKANPPAVT |
| | Cadherin- | FELTGETDNIFVIEREGLLYYNRALDRETRSTHNLQVAALDANGIIVEGPVPITIKVKDI |
| | 17, | NDNRPTFLQSKYEGSVRQNSRPGKPFLYVNATDLDDPATPNGQLYYQIVIQLPMINN |
| | Intestinal | VMYFQINNKTGAISLTREGSQELNPAKNPSYNLVISVKDMGGQSENSFSDTTSVDIIVT |
| | peptide- | ENIWKAPKPVEMVENSTDPHPIKITQVRWNDPGAQYSLVDKEKLPRFPFSIDQEGDIY |
| | associated | VTQPLDREEKDAYVFYAVAKDEYGKPLSYPLEIHVKVKDINDNPPTCPSPVTVFEVQ |
| | transporter | ENERLGNSIGTLTAHDRDEENTANSFLNYRIVEQTPKLPMDGLFLIQTYAGMLQLAK |
| | HPT-1; | QSLKKQDTPQYNLTIEVSDKDFKTLCFVQINVIDINDQIPIFEKSDYGNLTLAEDTNIGS |
| | Liver- | TILTIQATDADEPFTGSSKILYHIIKGDSEGRLGVDTDPHTNTGYVIIKKPLDFETAAVS |
| | intestine | NIVFKAENPEPLVFGVKYNASSFAKFTLIVTDVNEAPQFSQHVFQAKVSEDVAIGTKV |
| | cadherin | GNVTAKDPEGLDISYSLRGDTRGWLKIDHVTGEIFSVAPLDREAGSPYRVQVVATEV |
| | | GGSSLSSVSEFHLILMDVNDNPPRLAKDYTGLFFCHPLSAPGSLIFEATDDDQHLFRGP |
| | | HFTFSLGSGSLQNDWEVSKINGTHARLSTRHTEFEEREYVVLIRINDGGRPPLEGIVSL |
| | | PVTFCSCVEGSCFRPAGHQTGIPTVGMAVGILLTTLLVIGIILAVVFIRIKKDKGKDNV |
| | | ESAQASEVKPLRS |

The present disclosure provides anti-CDH17 antibodies and antigen-binding portions thereof. As non-limiting examples, the disclosure provides the anti-CDH17 heavy and light chain variable region amino acid sequences set forth as SEQ ID NOs: 2-9 in Table 3, and the variable heavy only (VHO) single domain antibodies set forth as SEQ ID NOs: 10-37 in Table 3.

In some embodiments, the present disclosure provides an anti-CDH17 antibody or an antigen-binding portion thereof comprising a heavy chain variable region comprising three Complementarity Determining Regions (CDRs), designated as HCDR1, HCDR2, and HCDR3, wherein the HCDR1, HCDR2, and HCDR3 are selected from:
SEQ ID NO: 65, SEQ ID NO: 66, and SEQ ID NO: 67; and
SEQ ID NO: 68, SEQ ID NO: 69, and SEQ ID NO: 70; respectively.

In some embodiments, the present disclosure provides an anti-CDH17 antibody or an antigen-binding portion thereof, comprising a light chain variable region comprising three CDRs, designated as LCDR1, LCDR2, and LCDR3, wherein the LCDR1, LCDR2, and LCDR3 are selected from:
SEQ ID NO: 62, SEQ ID NO: 63, and SEQ ID NO: 64; and
SEQ ID NO: 71, SEQ ID NO: 72, and SEQ ID NO: 73; respectively.

In some embodiments, the present disclosure provides an anti-CDH17 antibody or an antigen-binding portion thereof comprising a heavy chain variable region comprising three Complementarity Determining Regions (CDRs), designated as HCDR1, HCDR2, and HCDR3, wherein the HCDR1, HCDR2, and HCDR3 are selected from: SEQ ID NO: 65, SEQ ID NO: 66, and SEQ ID NO: 67; and SEQ ID NO: 68, SEQ ID NO: 69, and SEQ ID NO: 70; respectively; and a light chain variable region comprising three CDRs, designated as LCDR1, LCDR2, and LCDR3, wherein the LCDR1, LCDR2, and LCDR3 are selected from: SEQ ID NO: 62, SEQ ID NO: 63, and SEQ ID NO: 64; and SEQ ID NO: 71, SEQ ID NO: 72, and SEQ ID NO: 73; respectively.

In some embodiments, the disclosure provides an anti-CDH17 antibody or an antigen binding fragment thereof, comprising a heavy chain sequence comprising an amino acid sequence with at least 85% (e.g., 85%, 90%, 95%, 96%, 97%, 98%, 99%, or 100%) identity to any one of SEQ ID NOs: 3 and 5, or an antigen-binding portion thereof, and a light chain sequence comprising an amino acid sequence with at least 85% (e.g., 85%, 90%, 95%, 96%, 97%, 98%, 99%, or 100%) identity to any one of SEQ ID NOs: 2 and 6, or an antigen-binding portion thereof.

In some embodiments, the disclosure provides an anti-CDH17 antibody or an antigen binding fragment thereof, comprising a heavy chain sequence and a light chain sequence comprising: SEQ ID NOs: 3 and 2; SEQ ID NOs: 4 and 2; SEQ ID NOs: 5 and 6; SEQ ID NOs: 7 and 9; or SEQ ID NOs: 8 and 9; respectively.

In some embodiments, the present disclosure provides an anti-CDH17 antibody or an antigen-binding portion thereof, comprising at least one variable-heavy-chain-only single-domain or an antigen-binding portion thereof, wherein the at least one variable-heavy-chain-only single-domain comprises HCDR1, HCDR2, and HCDR3 selected from:
SEQ ID NO: 74, SEQ ID NO: 75, and SEQ ID NO: 76;
SEQ ID NO: 77, SEQ ID NO: 78, and SEQ ID NO: 79;
SEQ ID NO: 80, SEQ ID NO: 81, and SEQ ID NO: 82;
SEQ ID NO: 77, SEQ ID NO: 83, and SEQ ID NO: 84;
SEQ ID NO: 77, SEQ ID NO: 81, and SEQ ID NO: 85;
SEQ ID NO: 86, SEQ ID NO: 87, and SEQ ID NO: 88;
SEQ ID NO: 86, SEQ ID NO: 89, and SEQ ID NO: 90;
SEQ ID NO: 77, SEQ ID NO: 91, and SEQ ID NO: 92;
SEQ ID NO: 93, SEQ ID NO: 75, and SEQ ID NO: 94;
SEQ ID NO: 77, SEQ ID NO: 95, and SEQ ID NO: 96;
SEQ ID NO: 77, SEQ ID NO: 97, and SEQ ID NO: 98;
SEQ ID NO: 77, SEQ ID NO: 99, and SEQ ID NO: 100;
SEQ ID NO: 86, SEQ ID NO: 101, and SEQ ID NO: 102;
SEQ ID NO: 103, SEQ ID NO: 104, and SEQ ID NO: 105;
SEQ ID NO: 86, SEQ ID NO: 106, and SEQ ID NO: 107;
SEQ ID NO: 108, SEQ ID NO: 109, and SEQ ID NO: 110;
SEQ ID NO: 77, SEQ ID NO: 111, and SEQ ID NO: 112;
SEQ ID NO: 77, SEQ ID NO: 113, and SEQ ID NO: 114;
SEQ ID NO: 115, SEQ ID NO: 116, and SEQ ID NO: 117;
SEQ ID NO: 77, SEQ ID NO: 118, and SEQ ID NO: 119;
SEQ ID NO: 120, SEQ ID NO: 78, and SEQ ID NO: 121;
SEQ ID NO: 77, SEQ ID NO: 101, and SEQ ID NO: 122;
SEQ ID NO: 115, SEQ ID NO: 123, and SEQ ID NO: 124;
SEQ ID NO: 115, SEQ ID NO: 125, and SEQ ID NO: 126;
SEQ ID NO: 115, SEQ ID NO: 127, and SEQ ID NO: 128;
SEQ ID NO: 77, SEQ ID NO: 101, and SEQ ID NO: 129;
SEQ ID NO: 130, SEQ ID NO: 131, and SEQ ID NO: 132; and
SEQ ID NO: 115, SEQ ID NO: 133, and SEQ ID NO: 134; respectively.

In some embodiments, the present disclosure provides an anti-CDH17 antibody or an antigen-binding portion thereof, comprising at least one variable-heavy-chain-only single-domain or an antigen-binding portion thereof, wherein the at least one variable-heavy-chain-only (VHO) single-domain comprises an amino acid sequence with at least 85% (e.g., 85%, 90%, 95%, 96%, 97%, 98%, 99%, or 100%) identity to any one of SEQ ID NOs: 10-37 or an antigen-binding portion thereof.

The anti-CDH17 antibodies of the present disclosure encompass full length antibody comprising two heavy chains and two light chains. The antibodies can be human or humanized chimeric antibodies. Humanized antibodies, as used herein, include chimeric antibodies and CDR-grafted antibodies. Chimeric antibodies are antibodies that include a non-human antibody variable region linked to a human constant region. CDR-grafted antibodies are antibodies that include the CDRs from a non-human “donor” antibody linked to the framework region from a human “recipient” antibody. Exemplary human or humanized antibodies include IgG, IgM, IgE, IgA, and IgD antibodies. The present antibodies can be of any class (IgG, IgM, IgE, IgA, IgD, etc.) or isotype. For example, a human antibody can comprise an IgG Fc domain, such as at least one of isotypes, IgG1, IgG2, IgG3, or IgG4.

In some embodiments, the anti-CDH17 antibody or antigen-binding portion thereof is selected from the group consisting of a whole antibody, an antibody fragment, a human antibody, humanized antibody, chimeric antibody, a single chain antibody, a conjugate, an antibody mimetic, and a defucosylated antibody. In further examples, the anti-CDH17 antibody fragment is selected from the group consisting of a UniBody, a variable heavy only single domain antibody, and a Nanobody. For example, the anti-CDH17 antibody fragment is a Nanobody as noted in SEQ ID NOs: 10-37. In some examples, the anti-CDH17 antibody fragment is selected from the group consisting of a single domain VHH, a single domain VHO, an Affibody, a DARPin, an Anticalin, an Avimer, a Versa body, and a Duocalin.

### Anti-CD3 Antibodies

The present disclosure provides anti-CD3 antibodies and antigen-binding portions thereof. As non-limiting examples, the disclosure provides anti-CD3 amino acid sequences set forth as SEQ ID NOs: 38-44 in Table 4.

In some embodiments, the present disclosure provides an anti-CD3 antibody or an antigen-binding portion thereof comprising a heavy chain variable region comprising three Complementarity Determining Regions (CDRs), designated as HCDR1, HCDR2, and HCDR3, wherein the HCDR1, HCDR2, and HCDR3 are selected from:
SEQ ID NO: 135, SEQ ID NO: 136, and SEQ ID NO: 137;
SEQ ID NO: 135, SEQ ID NO: 138, and SEQ ID NO: 137; and
SEQ ID NO: 135, SEQ ID NO: 139, and SEQ ID NO: 137; respectively

In some embodiments, the present disclosure provides an anti-CD3 antibody or an antigen-binding portion thereof, comprising a light chain variable region comprising three CDRs, designated as LCDR1, LCDR2, and LCDR3, wherein the LCDR1, LCDR2, and LCDR3 are selected from:
SEQ ID NO: 140, SEQ ID NO: 141, and SEQ ID NO: 142; and
SEQ ID NO: 143, SEQ ID NO: 141, and SEQ ID NO: 142; respectively

In some embodiments, the present disclosure provides an anti-CD3 antibody or an antigen-binding portion thereof comprising a heavy chain variable region comprising three Complementarity Determining Regions (CDRs), designated as HCDR1, HCDR2, and HCDR3, wherein the HCDR1, HCDR2, and HCDR3 are selected from:
SEQ ID NO: 135, SEQ ID NO: 136, and SEQ ID NO: 137;
SEQ ID NO: 135, SEQ ID NO: 138, and SEQ ID NO: 137; and
SEQ ID NO: 135, SEQ ID NO: 139, and SEQ ID NO: 137; respectively; and a light chain variable region comprising three CDRs, designated as LCDR1, LCDR2, and LCDR3, wherein the LCDR1, LCDR2, and LCDR3 are selected from:
SEQ ID NO: 140, SEQ ID NO: 141, and SEQ ID NO: 142; and SEQ ID NO: 143, SEQ ID NO: 141, and SEQ ID NO: 142; respectively

In some embodiments, the disclosure provides an anti-CD3 antibody or an antigen binding fragment thereof comprising a heavy chain sequence comprising an amino acid sequence with at least 85% (e.g., 85%, 90%, 95%, 96%, 97%, 98%, 99%, or 100%) identity to any one of SEQ ID NOs: 39-41 or an antigen-binding portion thereof, and a light chain sequence comprising an amino acid sequence with at least 85% (e.g., 85%, 90%, 95%, 96%, 97%, 98%, 99%, or 100%) identity to any one of SEQ ID NOs: 43-44 or an antigen-binding portion thereof.

In some embodiments, the disclosure provides an anti-CD3 antibody or an antigen binding fragment thereof, comprising a heavy chain sequence and a light chain sequence comprising: SEQ ID NOs: 38 and 42; SEQ ID NOs: 38 and 43; SEQ ID NOs: 38 and 44; SEQ ID NOs: 39 and 42; SEQ ID NOs: 39 and 43; SEQ ID NOs: 39 and 44; SEQ ID NOs: 40 and 42; SEQ ID NOs: 40 and 43; SEQ ID NOs: 40 and 44; SEQ ID NOs: 41 and 42; SEQ ID NOs: 41 and 43; and SEQ ID NOs: 41 and 44; respectively.

The anti-CD3 antibodies of the present disclosure encompass full length antibody comprising two heavy chains and two light chains. The antibodies can be human or humanized antibodies.

In some embodiments, the anti-CD3 antibody or an antigen-binding portion thereof is selected from the group consisting of a whole antibody, an antibody fragment, a human antibody, humanized antibody, a single chain antibody, a conjugate, an antibody mimetic, and a defucosylated antibody. In further examples, the anti-CD3 antibody fragment is selected from the group consisting of a UniBody, a variable heavy only single domain antibody, and a Nanobody. In some examples, the anti-CD3 antibody fragment is selected from the group consisting of a single domain VHH, a single domain VHO, an Affibody, a DARPin, an Anticalin, an Avimer, a Versa body, and a Duocalin.

### Bispecific CD3×CDH17 Antibodies

In some embodiments, the present disclosure provides a bispecific antibody that simultaneously targets human CD3 and CDH17. In some embodiments, the present disclosure provides a CD3×CDH17 bispecific antibody without shielding domains. In some embodiments, the present disclosure provides a CD3×CDH17 bispecific antibody that has shielding domains that are removed by proteases and other in situ specific enzymes, which are found in the tumor microenvironment. Presence of shielding domains can reduce systemic toxicity of the CD3×CDH17 bispecific antibody. The CD3 and CDH17 targets have differential expression levels in pathological sites and normal tissues. The shielded CD3×CDH17 bispecific antibody remains inactive in normal tissue due to the inhibitory effects of the masking domain on the binding domains, e.g., the CDRs in the anti-CD3 and anti-CDH17 binding arms. The masking domains are cleaved off by proteases in disease sites and the shielded CD3×CDH17 bispecific antibody is converted to an active CD3×CDH17 bispecific antibody.

In some embodiments, the present disclosure provides a bispecific antibody that targets and binds to human CDH17 and CD3 simultaneously, has high affinity, and is capable of effectively blocking CDH17 proteins at the protein level. The bispecific antibody binds both CD3 and CDH17 proteins and binds to one protein without affecting the binding of the other protein, that is, having the ability to bind CD3 and CDH17 simultaneously. The bispecific antibody fills the gap that there is no antibody which simultaneously targets CD3 and CDH17. The bispecific antibody inhibits the proliferation of vascular endothelial cells, human lung cancer cells, human breast cancer cells, human pancreatic cancer cells, and human gastric cancer cells.

In some embodiments, the present disclosure provides a bispecific antibody comprising:
a first binding arm comprising:
a first heavy chain fusion protein comprising, from the N- to the C-terminus, a mask A, a protease cleavable linker A, and an IgG heavy chain or an antigen-binding portion thereof, and
a first light chain fusion protein comprising, from the N- to the C-terminus, a mask B, a protease cleavable linker B, and an IgG light chain or an antigen-binding portion thereof,
wherein the IgG heavy chain or antigen-binding portion thereof and the IgG light chain or antigen-binding portion thereof of the first binding arm are capable of targeting a CD3 associated pathway; and
a second binding arm comprising:
a second heavy chain fusion protein comprising, from the N- to the C-terminus, a mask C, a protease cleavable linker C, and an IgG heavy chain or an antigen-binding portion thereof; and
a second light chain fusion protein comprising, from the N- to the C-terminus, a mask D, a protease cleavable linker D, and an IgG light chain or an antigen-binding portion thereof,
wherein the IgG heavy chain or antigen-binding portion thereof and the IgG light chain or antigen-binding portion thereof of the second binding arm are capable of targeting a CDH17 associated pathway.
   The masks A-D can be the same or different from one another, and the protease cleavable linkers A-D are the same or different from one another.

In some embodiments, in a bispecific antibody disclosed herein,
the first binding arm comprises:
a first heavy chain fusion protein comprising, from the N- to the C-terminus, a signal sequence A-a mask A-a linker A-a protease cleavable linker A-a linker B-an IgG heavy chain or an antigen-binding portion thereof, and
a first light chain fusion protein comprising, from the N- to the C-terminus, a signal sequence B-a mask B-a linker C-a protease cleavable linker B-a linker D-an IgG light chain or an antigen-binding portion thereof,
wherein the IgG heavy chain or antigen-binding portion thereof and the IgG light chain or antigen-binding portion thereof of the first binding arm are capable of targeting a CD3 associated pathway; and
the second binding arm comprises:
a second heavy chain fusion protein comprising, from the N- to the C-terminus, a signal sequence C-a mask C-a linker E-a protease cleavable linker C-a linker F-an IgG heavy chain or an antigen-binding portion thereof; and
a second light chain fusion protein comprising, from the N- to the C-terminus, a signal sequence D-a mask D-a linker G-a protease cleavable linker D-a linker H-an IgG light chain or an antigen-binding portion thereof,
wherein the IgG heavy chain or antigen-binding portion thereof and the IgG light chain or antigen-binding portion thereof of the second binding arm are capable of targeting a CDH17 associated pathway.
   The signal sequences A-D can be the same or different from one another, and the linkers A-H are the same or different from one another.

In some embodiments, the bispecific antibody consists of two sets of light chain fusions and two sets of heavy chain fusions. In some embodiments, the bispecific antibody comprises a human IgG₁ heavy chain fusion comprising from the N- to the C-terminus: signal sequence A-shield A-linker A-protease sequence A-linker B-IgG₁ heavy chain; and a human IgG1 light chain fusion comprising amino acid sequences from the N- to the C-terminus, signal sequence B-shield B-linker B-protease sequence B-linker C-IgG1 light chain. Signal sequence A can be the same or different from Signal sequence B. Shield A can be the same or different from shield B. Linkers A, B, and C can be the same or different from one another. Protease sequence B can be same or different from protease sequence A.

The present disclosure provides a method for making a bispecific antibody disclosed herein, for example, using well established point mutations in the CH1, CH2, and CH3 domains via controlled Fab arm exchange or via co-expression. In some embodiment, all constructs for making the bispecific antibody are symmetric so that there is no preference for the selection of point mutations of the respective parental antibodies.

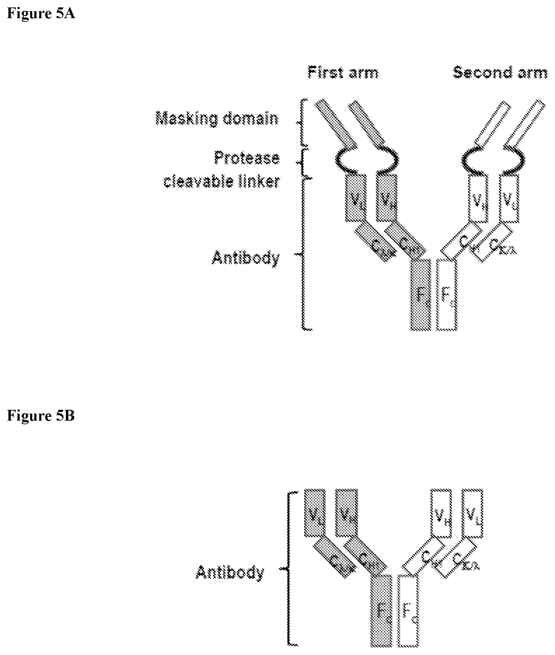

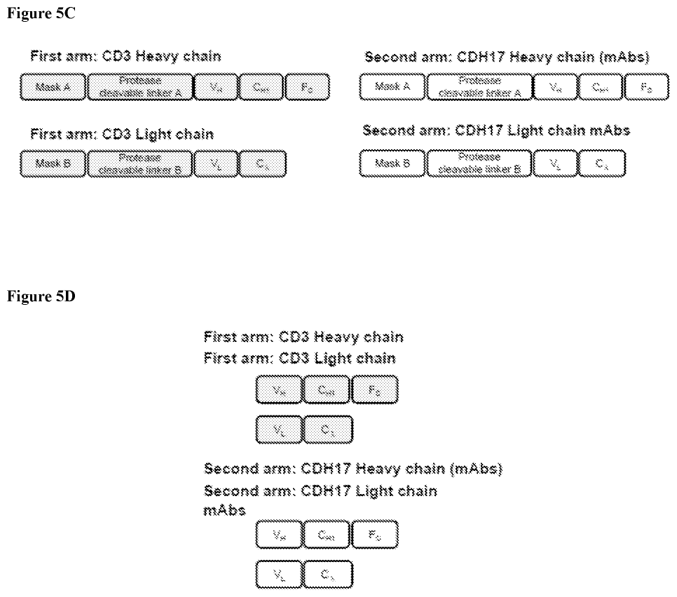

FIGS. 5-7 illustrate several formats of a bispecific antibody disclosed herein, with or without masking domains. FIG. 5A-5D illustrate the structure of the light chains and the heavy chains for the respective parental antibodies for making a bispecific antibody disclosed herein. As shown in FIG. 5A-5D, a shielded CD3×CDH17 bispecific antibody has two different sets of heavy chain and light chain pairing, which are indicated as “first arm” and “second arm.” A light chain fusion protein comprises from the N-terminus to the C-terminus: a mask domain B, a protease cleavable linker B, a light chain variable region VL, and a constant light chain CL. A heavy chain fusion protein comprises from the N-terminus to the C-terminus: a mask domain A, a protease cleavable linker A, a heavy chain variable region VH, a CH1 domain, and an Fc region. Different linkers can be put between the Mask A and protease cleavable linker A, protease cleavable linker A and VH, as well as CH1 and Fc domain. Different linkers can be placed between the Mask B and protease cleavable linker B, and protease cleavable linker B and VL. The heavy chain fusion protein and light chain fusion protein can have the same or different complementary mask domains and protease cleavable linkers.

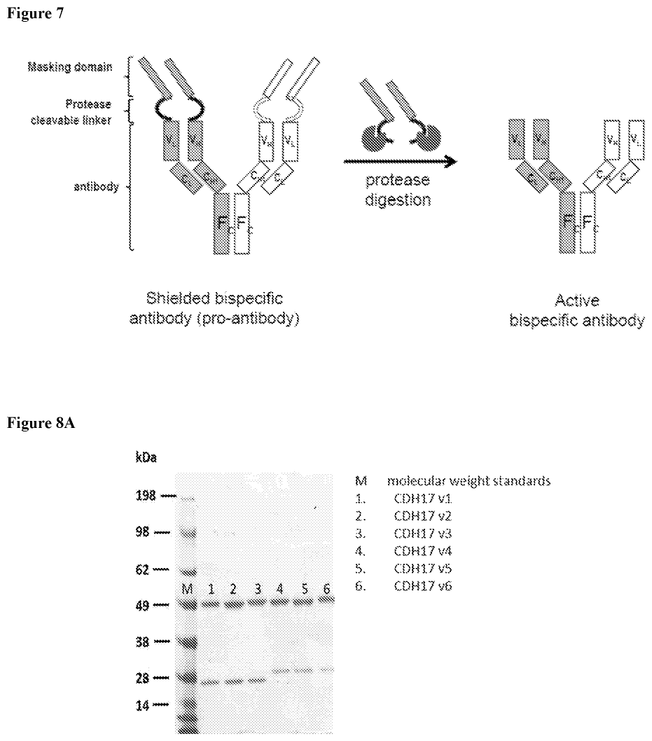

FIG. 7 is a schematic representation of removal of the masking domains from a CD3×CDH17 bispecific antibody. Proteases that are in higher concentrations in a tumor microenvironment can cut along the protease cleavable linkers to convert a shielded bispecific antibody to an active bispecific antibody.

Long term administration of anti-CD3 or anti-CDH17 biologics drugs pose a great risk factor for patients. In some embodiments, the present disclosure provides a combination of shields for a bispecific antibody that can block the Fab arm engagement to their respective epitopes. The shields can be fused to the heavy chain and/or the light chain domains. For example, the shields can block the Fab CDR regions from binding to the antigen via steric hindrance. The conversion of anti-CD3 and/or anti-CDH17 antibody arms into a shielded arm with the masking domains may increase the safety profile and therapeutic window of the respective arms in the bispecific antibody. In some embodiments, the present disclosure provides a shield or cap that masks CDH17 binding, wherein the shield or cap comprises an amino acid sequence selected from SEQ ID NOs: 47-50. In some embodiments, the present disclosure provides a shield or cap that masks CD3 binding, wherein the shield or cap comprises an amino acid sequence set forth in SEQ ID NO: 51. In some embodiments, the present disclosure provides a bispecific antibody comprising a protease substrate linker selected from SEQ ID NOs: 52-59.

In some embodiments of the bispecific antibody disclosed herein, the binding arm that targets CD3 has a binding valency of one. In some embodiments, the present disclosure provides a CD3×CDH17 bispecific antibody that preferably has a monovalent CD3-binding (i.e., one Fab arm binding to the epitope of CD3) arm. Bivalent CD3 binding is linked to excess of activation-induced cell death in effector cells that would limit the efficacy of T cell redirection. In addition, a bivalent CD3 redirection molecule can result in tumor antigen-independent immune effector cell activation that can increase systemic toxicity in the patient. Bivalent anti-CD3 agents can increase avidity in binding to CD3 and could lead to increase in cytokine release syndrome from off-tumor CD3 positive cells. The use of a high affinity of anti-CD3 antibody Fab arm can also increase toxicity. High affinity variants of anti-CD3 Fab or scFv are typically poorly tolerated in cynomolgus monkeys because of resulting extensive cytokine release. High affinity for CD3 could also shift a bispecific antibody biodistribution from tumors to CD3 rich tissue that leads to increased risk of cytokine release syndrome. Thus, it is highly desirable for the binding affinity of the anti-CD3 arm to be substantially lower than that of anti-CDH17 arm.

In some embodiments, the binding arm targeting a CDH17 associated pathway in bispecific antibody can be monovalent, bivalent, trivalent, tetravalent, etc. In some embodiments, the present disclosure provides a CD3×CDH17 bispecific antibody with CDH17 binding valency of 1-4, e.g., 1 or 2.

In some embodiments, the present disclosure provides a bispecific antibody comprising an arm targeting CD3, which comprises an amino acid sequence selected from SEQ ID NOs: 10-16, and comprising an arm targeting CDH17, which comprises an amino acid sequence selected from SEQ ID NOs: 2-9. Such bispecific antibodies are made by recombinant molecular biology techniques.

### Leader Sequences

In certain embodiments, a leader peptide is incorporated to drive the secretion of a shielded CD3×CDH17 bispecific antibody described herein into the cell culture supernatant as a secreted respective parental antibody protein. Any leader peptide for any known secreted proteins/peptides can be used.

As used herein, a “leader peptide,” “lead peptide,” or “signal peptide” includes a short peptide, usually 16-30 amino acids in length, that is present at the N-terminus of most of newly synthesized proteins that are destined towards the secretory pathway. Although lead peptides are extremely heterogeneous in sequence, and many prokaryotic and eukaryotic lead peptides are functionally interchangeable even between different species, the efficiency of protein secretion may be strongly determined by the sequence of the lead/signal peptide.

In certain embodiments, the leader peptide is from a protein residing either inside certain organelles (such as the endoplasmic reticulum, Golgi, or endosomes), secreted from the cell, or inserted into most cellular membranes.

In certain embodiments, the leader peptide is from a eukaryotic protein.

In certain embodiments, the leader peptide is from a secreted protein, e.g., a protein secreted outside a cell.

In certain embodiments, the leader peptide is from a transmembrane protein.

In certain embodiments, the leader peptide contains a stretch of amino acids that is recognized and cleaved by a signal peptidase.

In certain embodiments, the leader peptide does not contain a cleavage recognition sequence of a signal peptidase.

In certain embodiments, the leader peptide is a signal peptide for tissue plasminogen activator (tPA), herpes simplex virus glycoprotein D (HSV gD), a growth hormone, a cytokine, a lipoprotein export signal, CD2, CD3δ, CD3ε, CD3γ, CD3ζ, CD4, CD8α, CD19, CD28, 4-1BB or GM-CSFR, or S. cerevisiae mating factor α-1 signal peptide.

In some embodiments, a leader sequence as described herein may be a mammalian CD4 or CD8 leader sequence, including but not limited to, e.g., a human CD4 or CD8 leader sequence, a non-human primate CD4 or CD8 leader sequence, a rodent CD4 or CD8 leader sequence, and the like. In some embodiments, a CD4 or CD8 leader comprises an amino acid sequence having at least 75%, at least 80%, at least 85%, at least 90%, at least 95%, at least 98%, at least 99%, or 100%, amino acid sequence identity with the human CD4 or CD8 leader sequences.

### Anti-CD3 and Anti-CDH17 Antibodies and Fragments Applicable for Shielded CD3×CDH17 Bispecific Antibody Design

In some embodiments, the present disclosure provides anti-CD3 and anti-CDH17 antibodies and antigen-binding fragments thereof for CD3×CDH17 bispecific antibody designs. The fabs of the CD3 and CDH17 antibodies or antigen-binding fragments thereof can be attached to masking domains via protease-cleavage linker sequences to make shielded CD3×CDH17 bispecific antibodies disclosed herein.

The antibodies and fragments applicable for a shielded CD3×CDH17 bispecific antibody design of the present disclosure encompass full length antibodies comprising two heavy chains and two light chains. The antibodies can be human or humanized antibodies.

In some embodiments, the anti-Cadherin-17 or anti-CD3 antibody of the present disclosure is selected from the group consisting of a whole antibody, an antibody fragment, a humanized or human antibody, a single chain antibody, a conjugate, an antibody mimetic, and a defucosylated antibody. The anti-Cadherin-17 or anti-CD3 antibody fragment may be selected from the group consisting of a UniBody, a domain antibody, and a Nanobody. In some embodiments, the anti-Cadherin-17 or anti-CD3 antibody of the present disclosure is selected from the group consisting of a single domain VHH, a single domain VHO, an Affibody, a DARPin, an Anticalin, an Avimer, a Versabody, and a Duocalin.

Human antibody heavy and light chain sequences are provided that form the CDR binding regions in a bispecific antibody that bind to CD3 and CDH17, respectively. As non-limiting examples, the disclosure provides for heavy and light chain variable region amino acid sequences of anti-CDH17 antibodies set forth as SEQ ID NOs: 2-9 and the variable heavy only (VHO) single domain antibodies set forth as SEQ ID NOs: 10-37 in Table 3. As non-limiting examples, the disclosure provides for amino acid sequences of anti-CD3 antibodies set forth as SEQ ID NOs: 38-44 in Table 4.

TABLE 3

| Description | Amino Acid Sequence | CDRs |
|---|---|---|
| CDH17 LC1 | DIVMSQSPSSLAVSVGEKVTMSCKSSQSL | CDR1: |
| | LHSSNQKNYLAWYQQKPGQSPKVLIYW | KSSQSLLHSSNQKNY |
| | ASTRESGVPDRFTGSGSGTDFTLTITSVKS | LA |
| | EDLAVYYCQQYYSYPWTFGGGTRLEIK | (SEQ ID NO: 62) |
| | (SEQ ID NO: 2) | CDR2: |
| | | WASTRES |
| | | (SEQ ID NO: 63) |
| | | CDR3: |
| | | QQYYSYPWT |
| | | (SEQ ID NO: 64) |
| CDH17 HC1 | EVQLQQSVAELVKPGASVKMSCKVSGY | CDR1: |
| | TLTDHTIHWMKQRPEQGLEWIGYIYPRD | KVSGYTLTDHTIH |
| | GITGYNEKFKGKATLTADTSSSTAYMQL | (SEQ ID NO: 65) |
| | NSLTSEDSAVYFCARWGYSYRNYAYYY | CDR2: |
| | DYWGQGTTLTVSS (SEQ ID NO: 3) | GYNEKFK |
| | | (SEQ ID NO: 66) |
| | | CDR3: |
| | | ARWGYSYRNYAYY |
| | | YDY |
| | | (SEQ ID NO: 67) |
| CDH17 H2 HC0 | QVQLVQSGAEVKKPGSSVKVSCKVSGYT | |
| | FTDHTIHWMKQRPGQGLEWIGYIYPRDVI | |
| | TQYNEKFKGKVTLTADKSTSTAYMELSS | |
| | LRSEDTAVYLCARWGYFYGSRSYYFDY | |
| | WGQGTTVTVSS (SEQ ID NO: 4) | |
| CDH17 E2 HC | QVQLQQSDAELVKPGASVKISCKVSGYT | CDR1: |
| | FTDHTIHWMKQRPEQGLEWIGYIYPRDV | KVSGYTFTDHTIH |
| | ITQYNEKFKGKATLTADKSSSTAYMQLN | (SEQ ID NO: 68) |
| | SLTSEDSAVYLCARWGYFYGSRSYYFD | CDR2: |
| | YWGQGTTLTVSS (SEQ ID NO: 5) | QYNEKFK |
| | | (SEQ ID NO: 69) |
| | | CDR3: |
| | | ARWGYFYGSRSYYF |
| | | DY |
| | | (SEQ ID NO: 70) |
| CDH17 E2 LC | DIVMTQSQKFMSTTVGDRVNITCKASQN | CDR1: |
| | VGTAVAWYQQKPGQSPKLLIYSASNRYT | KASQNVGTAVA |
| | GVPDRFTGSGSGTDFTLTITNMQSKDLA | (SEQ ID NO: 71) |
| | DYFCQQYSSYPWTFGGGTKLEIK (SEQ | CDR2: |
| | ID NO: 6) | GVPDRFT |
| | | (SEQ ID NO: 72) |
| | | CDR3: |
| | | QQYSSYPWT |
| | | (SEQ ID NO: 73) |
| CDH17 M1 HC | EVQLQQSVAELVKPGASVKMSCKVSGYT | |
| | LTDHTIHMKQRPEQGLEWIGYIYPRDGIT | |
| | GYNEKFKGKATLTADTSSSTAYMQLNSL | |
| | TSEDSAVYFCARWGYSYRNYAYYYDYW | |
| | GQGTTLTVSS (SEQ ID NO: 7) | |
| CDH17 M3 HC | EVQLVESGGGLVKPGGSLKLSCAASGFSF | |
| | SDYYMYWVRQAPEKRLEWVASISFDGT | |
| | YTYYTDRVKGRFTISRDNAKNNLYLQMS | |
| | SLKSEDTAMYYCARDRPAWFPYWGQGT | |
| | LVTVSA (SEQ ID NO: 8) | |
| CDH17 M3 LC | DVLMTQIPLSLTVSLGDQASISCRSSQSIV | |
| | HSNGNTYLEWYLQRPGPSPKLLIYKVSN | |
| | RFSGVPDRFSGSGSGSGTDFTLKISRVEAE | |
| | DLGVYYCFQGSHVPLTFGAGTKLELK | |
| | (SEQ ID NO: 9) | |
| TS00349 | EVQLLESGGGLVQPGGSLRLSCAASGFTF | CDR1: |
| | SDYWMHWVRQAPGKGLEWVSAIWSSG | DYWMH |
| | GSTYYADSVKGRFTISRDNSKNTLYLQM | (SEQ ID NO: 74) |
| | NSLRAEDTAVYYCAKSPTLYDDFDYWG | CDR2: |
| | QGTLVTVSS (SEQ ID NO: 10) | AIWSSGGSTYYADSV |
| | | KG |
| | | (SEQ ID NO: 75) |
| | | CDR3: |
| | | SPTLYDDFDY |
| | | (SEQ ID NO: 76) |
| TS00369 | EVQLLESGGGLVQPGGSLRLSCAASGFTF | CDR1: |
| | SDYWMSWVRQAPGKGLEWVSNISGSGS | DYWMS |
| | STYYADSVKGRFTISRDNSKNTLYLQMN | (SEQ ID NO: 77) |
| | SLRAEDTAVYYCAKTSQWLARERSSEF | CDR2: |
| | DYWGQGTLVTVSS (SEQ ID NO: 11) | NISGSGSSTYYADSV |
| | | KG |
| | | (SEQ ID NO: 78) |
| | | CDR3: |
| | | TSQWLARERSSEFDY |
| | | (SEQ ID NO: 79) |
| TS00347 | EVQLLESGGGLVQPGGSLRLSCAASGFTF | CDR1: |
| | STYAMSWVRQAPGKGLEWVSNISGSGG | TYAMS |
| | STYYADSVKGRFTISRDNSKNTLYLQMN | (SEQ ID NO: 80) |
| | SLRAEDTAVYYCAKEQQWVPGFGFDY | CDR2: |
| | WGQGTLVTVSS (SEQ ID NO: 12) | NISGSGGSTYYADSV |
| | | KG |
| | | (SEQ ID NO: 81) |
| | | CDR3: |
| | | EQQWVPGFGFDY |
| | | (SEQ ID NO: 82) |
| TS00366 | EVQLLESGGGLVQPGGSLRLSCAASGFTF | CDR1: |
| | SDYWMSWVRQAPGKGLEWVSNINGDG | DYWMS |
| | GSTYYADSVKGRFTISRDNSKNTLYLQM | (SEQ ID NO: 77) |
| | NSLRAEDTAVYYCAKTSTFYKQDKFDY | CDR2: |
| | WGQGTLVTVSS (SEQ ID NO: 13) | NINGDGGSTYYADS |
| | | VKG |
| | | (SEQ ID NO: 83) |
| | | CDR3: |
| | | TSTFYKQDKFDY |
| | | (SEQ ID NO: 84) |
| TS00364 | EVQLLESGGGLVQPGGSLRLSCAASGFTF | CDR1: |
| | SDYWMSWVRQAPGKGLEWVSNISGSG | DYWMS |
| | GSTYYADSVKGRFTISRDNSKNTLYLQM | (SEQ ID NO: 77) |
| | NSLRAEDTAVYYCAKTSDWLLHDADLH | CDR2: |
| | FDYWGQGTLVTVSS (SEQ ID NO: 14) | NISGSGGSTYYADSV |
| | | KG |
| | | (SEQ ID NO: 81) |
| | | CDR3: |
| | | TSDWLLHDADLHFD |
| | | Y |
| | | (SEQ ID NO: 85) |
| TS00363 | EVQLLESGGGLVQPGGSLRLSCAASGFTF | CDR1: |
| | STYWMSWVRQAPGKGLEWVSNISQDG | TYWMS |
| | GSTYYADSVKGRFTISRDNSKNTLYLQM | (SEQ ID NO: 86) |
| | NSLRAEDTAVYYCAKTTDWLQDKVDE | CDR2: |
| | AFDYWGQGTLVTVSS (SEQ ID NO: 15) | NISQDGGSTYYADSV |
| | | KG |
| | | (SEQ ID NO: 87) |
| | | CDR3: |
| | | TTDWLQDKVDEAFD |
| | | Y |
| | | (SEQ ID NO: 88) |
| TS00341 | EVQLLESGGGLVQPGGSLRLSCAASGFTF | CDR1: |
| | STYWMSWVRQAPGKGLEWVSVINQDG | TYWMS |
| | SSTYYADSVKGRFTISRDNSKNTLYLQM | (SEQ ID NO: 86) |
| | NSLRAEDTAVYYCAKDELKSEWDFDY | CDR2: |
| | WGQGTLVTVSS (SEQ ID NO: 16) | VINQDGSSTYYADSV |
| | | KG |
| | | (SEQ ID NO: 89) |
| | | CDR3: |
| | | DELKSEWDFDY |
| | | (SEQ ID NO: 90) |
| TS00357 | EVQLLESGGGLVQPGGSLRLSCAASGFTF | CDR1: |
| | SDYWMSWVRQAPGKGLEWVSNISSSGS | DYWMS |
| | STYYADSVKGRFTISRDNSKNTLYLQMN | (SEQ ID NO: 77) |
| | SLRAEDTAVYYCAKTSDWLSGKIDEHF | CDR2: |
| | DYWGQGTLVTVSS (SEQ ID NO: 17) | NISSSGSSTYYADSV |
| | | KG |
| | | (SEQ ID NO: 91) |
| | | CDR3: |
| | | TSDWLSGKIDEHFDY |
| | | (SEQ ID NO: 92) |
| TS00352 | EVQLLESGGGLVQPGGSLRLSCAASGFTF | CDR1: |
| | SSYWMHWVRQAPGKGLEWVSAIWSSG | SYWMH |
| | GSTYYADSVKGRFTISRDNSKNTLYLQM | (SEQ ID NO: 93) |
| | NSLRAEDTAVYYCAKSKSLHEDFDYWG | CDR2: |
| | QGTLVTVSS (SEQ ID NO: 18) | AIWSSGGSTYYADSV |
| | | KG |
| | | (SEQ ID NO: 75) |
| | | CDR3: |
| | | SKSLHEDFDY |
| | | (SEQ ID NO: 94) |
| TS00358 | EVQLLESGGGLVQPGGSLRLSCAASGFTF | CDR1: |
| | SDYWMSWVRQAPGKGLEWVSNISGDG | DYWMS |
| | GSTYYADSVKGRFTISRDNSKNTLYLQM | (SEQ ID NO: 77) |
| | NSLRAEDTAVYYCAKTTDLLQGAFDYW | CDR2: |
| | GQGTLVTVSS (SEQ ID NO: 19) | NISGDGGSTYYADSV |
| | | KG |
| | | (SEQ ID NO: 95) |
| | | CDR3: |
| | | TTDLLQGAFDY |
| | | (SEQ ID NO: 96) |
| TS00362 | EVQLLESGGGLVQPGGSLRLSCAASGFTF | CDR1: |
| | SDYWMSWVRQAPGKGLEWVSNISSDGG | DYWMS |
| | STYYADSVKGRFTISRDNSKNTLYLQMN | (SEQ ID NO: 77) |
| | SLRAEDTAVYYCAKTTTWLLSQHDKTS | CDR2: |
| | FDYWGQGTLVTVSS (SEQ ID NO: 20) | NISSDGGSTYYADSV |
| | | KG |
| | | (SEQ ID NO: 97) |
| | | CDR3: |
| | | TTTWLLSQHDKTSFD |
| | | Y |
| | | (SEQ ID NO: 98) |
| TS00365 | EVQLLESGGGLVQPGGSLRLSCAASGFTF | CDR1: |
| | SDYWMSWVRQAPGKGLEWVSNINQDG | DYWMS |
| | GSTYYADSVKGRFTISRDNSKNTLYLQM | (SEQ ID NO: 77) |
| | NSLRAEDTAVYYCAKTSDWLLVQFDY | CDR2: |
| | WGQGTLVTVSS (SEQ ID NO: 21) | (SEQ ID NO: 99) |
| | | CDR3: |
| | | TSDWLLVQFDY |
| | | (SEQ ID NO: 100) |
| TS00344 | EVQLLESGGGLVQPGGSLRLSCAASGFTF | CDR1: |
| | STYWMSWVRQAPGKGLEWVSAISGDGS | TYWMS |
| | STYYADSVKGRFTISRDNSKNTLYLQMNS | (SEQ ID NO: 86) |
| | LRAEDTAVYYCAKTWKSPKFDYWGQGT | CDR2: |
| | LVTVSS (SEQ ID NO: 22) | AISGDGSSTYYADSV |
| | | KG |
| | | (SEQ ID NO: 101) |
| | | CDR3: |
| | | TWKSPKFDY |
| | | (SEQ ID NO: 102) |
| TS00372 | EVQLLESGGGLVQPGGSLRLSCAASGFTF | CDR1: |
| | SSYWMSWVRQAPGKGLEWVSGINYSGS | SYWMS |
| | STYYADSVKGRFTISRDNSKNTLYLQMN | (SEQ ID NO: 103) |
| | SLRAEDTAVYYCAKWSWLGSDKADFD | CDR2: |
| | YWGQGTLVTVSS (SEQ ID NO: 23) | GINYSGSSTYYADSV |
| | | KG |
| | | (SEQ ID NO: 104) |
| | | CDR3: |
| | | WSWLGSDKADFDY |
| | | (SEQ ID NO: 105) |
| TS00374 | EVQLLESGGGLVQPGGSLRLSCAASGFTF | CDR1: |
| | STYWMSWVRQAPGKGLEWVSGINQDG | TYWMS |
| | SSTYYADSVKGRFTISRDNSKNTLYLQM | (SEQ ID NO: 86) |
| | NSLRAEDTAVYYCAKWHWAGSSQQFD | CDR2: |
| | YWGQGTLVTVSS (SEQ ID NO: 24) | GINQDGSSTYYADSV |
| | | KG |
| | | (SEQ ID NO: 106) |
| | | CDR3: |
| | | WHWAGSSQQFDY |
| | | (SEQ ID NO: 107) |
| TS00379 | EVQLLESGGGLVQPGGSLRLSCAASGFTF | CDR1: |
| | SSYGMSWVRQAPGKGLEWVSNINQSGS | SYGMS |
| | STYYADSVKGRFTISRDNSKNTLYLQMN | (SEQ ID NO: 108) |
| | SLRAEDTAVYYCAKHSHWAHRVLQSFD | CDR2: |
| | YWGQGTLVTVSS (SEQ ID NO: 25) | NINQSGSSTYYADSV |
| | | KG |
| | | (SEQ ID NO: 109) |
| | | CDR3: |
| | | HSHWAHRVLQSFDY |
| | | (SEQ ID NO: 110) |
| TS00342 | EVQLLESGGGLVQPGGSLRLSCAASGFTF | CDR1: |
| | SDYWMSWVRQAPGKGLEWVSNISQSG | DYWMS |
| | GSTYYADSVKGRFTISRDNSKNTLYLQM | (SEQ ID NO: 77) |
| | NSLRAEDTAVYYCAKDTPGPRWDFDY | CDR2: |
| | WGQGTLVTVSS (SEQ ID NO: 26) | NISQSGGSTYYADSV |
| | | KG |
| | | (SEQ ID NO: 111) |
| | | CDR3: |
| | | DTPGPRWDFDY |
| | | (SEQ ID NO: 112) |
| TS00343 | EVQLLESGGGLVQPGGSLRLSCAASGFTF | CDR1: |
| | SDYWMSWVRQAPGKGLEWVSAIKGDG | DYWMS |
| | SSTYYADSVKGRFTISRDNSKNTLYLQM | (SEQ ID NO: 77) |
| | NSLRAEDTAVYYCAKEEWRPYSFDYWG | CDR2: |
| | QGTLVTVSS (SEQ ID NO: 27) | AIKGDGSSTYYADSV |
| | | KG |
| | | (SEQ ID NO: 113) |
| | | CDR3: |
| | | EEWRPYSFDY |
| | | (SEQ ID NO: 114) |
| TS00345 | EVQLLESGGGLVQPGGSLRLSCAASGFTF | CDR1: |
| | SNYWMSWVRQAPGKGLEWVSAIKQDG | NYWMS |
| | SSTYYADSVKGRFTISRDNSKNTLYLQM | (SEQ ID NO: 115) |
| | NSLRAEDTAVYYCAKEEWRDYDFDYW | CDR2: |
| | GQGTLVTVSS (SEQ ID NO: 28) | AIKQDGSSTYYADSV |
| | | KG |
| | | (SEQ ID NO: 116) |
| | | CDR3: |
| | | EEWRDYDFDY |
| | | (SEQ ID NO: 117) |
| TS00346 | EVQLLESGGGLVQPGGSLRLSCAASGFTF | CDR1: |
| | SDYWMSWVRQAPGKGLEWVSAIKQDG | DYWMS |
| | GSTYYADSVKGRFTISRDNSKNTLYLQM | (SEQ ID NO: 77) |
| | NSLRAEDTAVYYCAKETWEAHWDFDY | CDR2: |
| | WGQGTLVTVSS (SEQ ID NO: 29) | AIKQDGGSTYYADS |
| | | VKG |
| | | (SEQ ID NO: 118) |
| | | CDR3: |
| | | ETWEAHWDFDY |
| | | (SEQ ID NO: 119) |
| TS00351 | EVQLLESGGGLVQPGGSLRLSCAASGFTF | CDR1: |
| | SSYAMSWVRQAPGKGLEWVSNISGSGSS | SYAMS |
| | TYYADSVKGRFTISRDNSKNTLYLQMNS | (SEQ ID NO: 120) |
| | LRAEDTAVYYCAKESQHSPGWGFDYW | CDR2: |
| | GQGTLVTVSS (SEQ ID NO: 30) | NISGSGSSTYYADSV |
| | | KG |
| | | (SEQ ID NO: 78) |
| | | CDR3: |
| | | ESQHSPGWGFDY |
| | | (SEQ ID NO: 121) |
| TS00355 | EVQLLESGGGLVQPGGSLRLSCAASGFTF | CDR1: |
| | SDYWMSWVRQAPGKGLEWVSAISGDGS | DYWMS |
| | STYYADSVKGRFTISRDNSKNTLYLQMN | (SEQ ID NO: 77) |
| | SLRAEDTAVYYCAKETWGTYDFDYWG | CDR2: |
| | QGTLVTVSS (SEQ ID NO: 31) | AISGDGSSTYYADSV |
| | | KG |
| | | (SEQ ID NO: 101) |
| | | CDR3: |
| | | ETWGTYDFDY |
| | | (SEQ ID NO: 122) |
| TS00361 | EVQLLESGGGLVQPGGSLRLSCAASGFTF | CDR1: |
| | SNYWMSWVRQAPGKGLEWVSNINGDG | NYWMS |
| | SSTYYADSVKGRFTISRDNSKNTLYLQM | (SEQ ID NO: 115) |
| | NSLRAEDTAVYYCAKTTSWLSRQDPDF | CDR2: |
| | DYWGQGTLVTVSS (SEQ ID NO: 32) | NINGDGSSTYYADSV |
| | | KG |
| | | (SEQ ID NO: 123) |
| | | CDR3: |
| | | TTSWLSRQDPDFDY |
| | | (SEQ ID NO: 124) |
| TS00370 | EVQLLESGGGLVQPGGSLRLSCAASGFTF | CDR1: |
| | SNYWMSWVRQAPGKGLEWVSNISSDGG | NYWMS |
| | STYYADSVKGRFTISRDNSKNTLYLQMN | (SEQ ID NO: 115) |
| | SLRAEDTAVYYCAKTTTWLTYRQGHG | CDR2: |
| | FDYWGQGTLVTVSS (SEQ ID NO: 33) | NISSDGGSTYYADSV |
| | | KG |
| | | (SEQ ID NO: 125) |
| | | CDR3: |
| | | TTTWLTYRQGHGFD |
| | | Y |
| | | (SEQ ID NO: 126) |
| TS00371 | EVQLLESGGGLVQPGGSLRLSCAASGFTF | CDR1: |
| | SNYWMSWVRQAPGKGLEWVSGINGSG | NYWMS |
| | GSTYYADSVKGRFTISRDNSKNTLYLQM | (SEQ ID NO: 115) |
| | NSLRAEDTAVYYCAKWHWYGQEKKAF | CDR2: |
| | DYWGQGTLVTVSS (SEQ ID NO: 34) | GINGSGGSTYYADSV |
| | | KG |
| | | (SEQ ID NO: 127) |
| | | CDR3: |
| | | WHWYGQEKKAFDY |
| | | (SEQ ID NO: 128) |
| TS00377 | EVQLLESGGGLVQPGGSLRLSCAASGFTF | CDR1: |
| | SDYWMSWVRQAPGKGLEWVSAISGDGS | DYWMS |
| | STYYADSVKGRFTISRDNSKNTLYLQMN | (SEQ ID NO: 77) |
| | SLRAEDTAVYYCAKEDWRPGFDYWGQ | CDR2: |
| | GTLVTVSS (SEQ ID NO: 35) | AISGDGSSTYYADSV |
| | | KG |
| | | (SEQ ID NO: 101) |
| | | CDR3: |
| | | EDWRPGFDY |
| | | (SEQ ID NO: 129) |
| TS00378 | EVQLLESGGGLVQPGGSLRLSCAASGFTF | CDR1: |
| | SNYAMSWVRQAPGKGLEWVSAISGSGG | NYAMS |
| | STYYADSVKGRFTISRDNSKNTLYLQMN | (SEQ ID NO: 130) |
| | SLRAEDTAVYYCAKFPFSHDPFDYWGQ | CDR2: |
| | GTLVTVSS (SEQ ID NO: 36) | AISGSGGSTYYADSV |
| | | KG |
| | | (SEQ ID NO: 131) |
| | | CDR3: |
| | | FPFSHDPFDY |
| | | (SEQ ID NO: 132) |
| TS00380 | EVQLLESGGGLVQPGGSLRLSCAASGFTF | CDR1: |
| | SNYWMSWVRQAPGKGLEWVSGISQSGS | NYWMS |
| | STYYADSVKGRFTISRDNSKNTLYLQMN | (SEQ ID NO: 115) |
| | SLRAEDTAVYYCAKWQFASWRGDKDF | CDR2: |
| | DYWGQGTLVTVSS (SEQ ID NO: 37) | GISQSGSSTYYADSV |
| | | KG |
| | | (SEQ ID NO: 133) |
| | | CDR3: |
| | | WQFASWRGDKDFD |
| | | Y |
| | | (SEQ ID NO: 134) |
| TS00376 | EVQLLESGGGLVQPGGSLRLSCAASGFTF | CDR1: DYWMS (SEQ |
| | SDYWMSWVRQAPGKGLEWVSGINSSG | ID NO: 77) |
| | GSTYYADSVKGRFTISRDNSKNTLYLQM | CDR2: |
| | NSLRAEDTAVYYCAKWQFVTQPGHKA | GINSSGGSTYYADS |
| | FDYWGQGTLVTVSS (SEQ ID NO: 145) | VKG |
| | | (SEQ ID NO: 146) |
| | | CDR3: |
| | | WQFVTQPGHKAFD |
| | | Y |
| | | (SEQ ID NO: 147) |

TABLE 4

| Description | Amino Acid Sequence | CDRs |
|---|---|---|
| Cris-7-VH0 | QVQLQQSGAELARPGASVKMSCKASGYT | |
| | FTRSTMHWVKQRPGQGLEWIGYINPSSA | |
| | YTNYNQKFKDKATLTADKSSSTAYMQLS | |
| | SLTSEDSAVYYCASPQVHYDYNGFPYWG | |
| | QGTLVTVSA (SEQ ID NO: 38) | |
| Cris-7-VH1 | QVQLVQSGAEVKKPGSSVKVSCKASGYT | CDR1: |
| | FTRSTMHWVRQAPGQGLEWIGYINPSSA | RSTMH |
| | YTNYAQKFQGRATLTADESTSTAYMELS | (SEQ ID NO: 135) |
| | SLRSEDTAVYYCASPQVHYDYNGFPYW | CDR2: |
| | GQGTLVTVSS (SEQ ID NO: 39) | YINPSSAYTNYAQKF |
| | | QG |
| | | (SEQ ID NO: 136) |
| | | CDR3: |
| | | PQVHYDYNGFPY |
| | | (SEQ ID NO: 137) |
| Cris-7-VH2 | EVQLVESGGGLVQPGRSLRLSCTASGYTF | CDR1: |
| | TRSTMHWVRQAPGKGLEWVGYINPSSA | RSTMH |
| | YTNYAASFKGRATLSADGSKSIAYLQMN | (SEQ ID NO: 135) |
| | SLKTEDTAVYYCASPQVHYDYNGFPYW | CDR2: |
| | GQGTLVTVSS (SEQ ID NO: 40) | YINPSSAYTNYAASF |
| | | KG |
| | | (SEQ ID NO: 138) |
| | | CDR3: |
| | | PQVHYDYNGFPY |
| | | (SEQ ID NO: 137) |
| Cris-7-VH3 | QVQLVQSGSELKKPGASVKMSCKASGYT | CDR1: |
| | FTRSTMHWVRQAPGQGLEWMGYINPSS | RSTMH |
| | AYTNYAQGFTGRAVLSADTSVSTAYLQL | (SEQ ID NO: 135) |
| | CSLKAEDTAVYYCASPQVHYDYNGFPY | CDR2: |
| | WGQGTLVTVSS (SEQ ID NO: 41) | YINPSSAYTNYAQGF |
| | | TG |
| | | (SEQ ID NO: 139) |
| | | CDR3: |
| | | PQVHYDYNGFPY |
| | | (SEQ ID NO: 137) |
| Cris-7-VL0 | QVVLTQSPAIMSAFPGEKVTMTCSASSSV | |
| | SYMNWYQQKSGTSPKRWIYDSSKLASGV | |
| | PARFSGSGSGTSYSLTISSMETEDAATYYC | |
| | QQWSRNPPTFGGGTKLQIT (SEQ ID NO: | |
| | 42) | |
| Cris-7-VL1 | DIQMTQSPSSLSASVGDRVTITCRASSSVS | CDR1: |
| | YMNWYQQKPGKAPKRLIYDSSKLASGV | RASSSVSYMN |
| | PSRFSGSGSGTEFTLTISSLQPEDFATYYC | (SEQ ID NO: 140) |
| | QQWSRNPPTFGQGTKLEIK (SEQ ID NO: | CDR2: |
| | 43) | DSSKLAS |
| | | (SEQ ID NO: 141) |
| | | CDR3: |
| | | QQWSRNPPT |
| | | (SEQ ID NO: 142) |
| Cris-7-VL2 | AIRLTQSPFSLSASVGDRVTITCSASSSVSY | CDR1: |
| | MNWYQQKPAKAPKRWIYDSSKLASGVP | SASSSVSYMN |
| | SRFSGSGSGTDYTLTISSLQPEDAATYYC | (SEQ ID NO: 143) |
| | QQWSRNPPTFGQGTKLEIK (SEQ ID NO: | CDR2: |
| | 44) | DSSKLAS |
| | | (SEQ ID NO: 141) |
| | | CDR3: |
| | | QQWSRNPPT |
| | | (SEQ ID NO: 142) |
| SP34 HC | EVQLVESGGGLVQPGGSLKLSCAASGFTF | |
| | NTYAMNWVRQAPGKGLEWVARIRSKYN | |
| | NYATYYADSVKDRFTISRDDSKNTAYLQ | |
| | MNNLKTEDTAVYYCVRHGNFGNSYVSW | |
| | FAYWGQGTLVTVSS (SEQ ID NO: 45) | |
| SP34 LC | QTVVTQEPSLTVSPGGTVTLTCRSSTGAV | |
| | PGTPARFSGSLLGGKAALTLSGVQPEDEA | |
| | TTSNYANWVQQKPGQAPRGLIGGTNKRA | |
| | EYYCALWYSNLWVFGGGTKLTVL (SEQ | |
| | ID NO: 46) | |

The present disclosure describes a monoclonal antibody, or an antigen-binding portion thereof, an antibody fragment, or an antibody mimetic that binds an epitope on human Cadherin-17 recognized by an antibody comprising a heavy chain variable region comprising an amino acid sequence selected from SEQ ID NOs: 3, 4, 5, 7, and 8 and a light chain variable region comprising an amino acid sequence selected from SEQ ID NOs: 2, 6, and 9.

The present disclosure provides a monoclonal antibody or an antigen binding portion thereof, an antibody fragment, or an antibody mimetic that binds an epitope on human Cadherin-17 recognized by an antibody comprising a heavy chain variable region and a light chain variable region selected respectively from the group consisting of the heavy chain variable region amino acid sequence set forth in SEQ ID NO: 3 and the light chain variable region amino acid sequence set forth in SEQ ID NO: 2; the heavy chain variable region amino acid sequence set forth in SEQ ID NO: 4 and the light chain variable region amino acid sequence set forth in SEQ ID NO: 2; the heavy chain variable region amino acid sequence set forth in SEQ ID NO: 5 and the light chain variable region amino acid sequence set forth in SEQ ID NO: 6; the heavy chain variable region amino acid sequence set forth in SEQ ID NO: 7 and the light chain variable region amino acid sequence set forth in SEQ ID NO: 9; and the heavy chain variable region amino acid sequence set forth in SEQ ID NO: 8 and the light chain variable region amino acid sequence set forth in SEQ ID NO: 9.

The present disclosure provides a monoclonal antibody or an antigen binding portion thereof, an antibody fragment, or an antibody mimetic that binds an epitope on human CD3 recognized by an antibody comprising a heavy chain variable region and a light chain variable region selected respectively from the group consisting of the heavy chain variable region amino acid sequence set forth in SEQ ID NO: 38 and the light chain variable region amino acid sequence set forth in SEQ ID NO: 42; the heavy chain variable region amino acid sequence set forth in SEQ ID NO: 39 and the light chain variable region amino acid sequence set forth in SEQ ID NO: 43; the heavy chain variable region amino acid sequence set forth in SEQ ID NO: 39 and the light chain variable region amino acid sequence set forth in SEQ ID NO: 44; the heavy chain variable region amino acid sequence set forth in SEQ ID NO: 40 and the light chain variable region amino acid sequence set forth in SEQ ID NO: 43; the heavy chain variable region amino acid sequence set forth in SEQ ID NO: 40 and the light chain variable region amino acid sequence set forth in SEQ ID NO: 44; the heavy chain variable region amino acid sequence set forth in SEQ ID NO: 41 and the light chain variable region amino acid sequence set forth in SEQ ID NO: 43; the heavy chain variable region amino acid sequence set forth in SEQ ID NO: 41 and the light chain variable region amino acid sequence set forth in SEQ ID NO: 43; and the heavy chain variable region amino acid sequence set forth in SEQ ID NO: 41 and the light chain variable region amino acid sequence set forth in SEQ ID NO: 44.

In some embodiments, the disclosure provides an isolated nucleic acid molecule encoding the heavy and/or light chain of the anti-Cadherin-17 antibody or antigen-binding portion thereof disclosed herein. In some embodiments, the disclosure provides an expression vector comprising such a nucleic acid, and a host cell comprising such expression vector or nucleic acid.

In some embodiments, the present disclosure provides a nucleic acid encoding the heavy and/or light chain of an anti-CD3 antibody or antigen binding portion thereof of the disclosure. In some embodiments, the present disclosure provides an expression vector comprising such nucleic acid, and a host cell comprising such expression vector or nucleic acid.

In some embodiments, the present disclosure provides a hybridoma expressing an anti-CDH17 antibody or antigen binding portion thereof. In some embodiments, the present disclosure provides a hybridoma expressing an anti-CD3 antibody or antigen binding portion thereof.

In some embodiments, the present disclosure provides a method of making the anti-Cadherin-17 or anti-CD3 antibodies of the disclosure, comprising immunizing an animal with a Cadherin-17 or CD3 peptide; Cadherin-17 or CD3 domain; or Cadherin-17 or CD3 protein; recovering mRNA from the B cells of said animal; and converting said mRNA to CDNA.

In some embodiments, the present disclosure provides a method for preparing an anti-Cadherin-17 antibody or an anti-CD3 antibody, said method comprising the steps of obtaining a host cell that contains one or more nucleic acid molecules encoding the anti-Cadherin-17 antibody or an anti-CD3 antibody of this disclosure; growing the host cell in a host cell culture; providing host cell culture conditions wherein the one or more nucleic acid molecules are expressed; and recovering the antibody from the host cell or from the host cell culture.

In some embodiments, the present disclosure provides a method of expressing cDNA encoding anti-Cadherin-17 antibodies or anti-CD3 monoclonal antibodies or an antigen binding portion thereof, an antibody fragment, or an antibody mimetic in phages such that the anti-Cadherin-17 antibodies or anti-CD3 antibodies encoded by said cDNA are presented on the surface of said phages; selecting phages that present anti-Cadherin-17 antibodies or anti-CD3 antibodies; recovering nucleic acid molecules from said selected phages that encode said anti-Cadherin-17 antibodies and said or anti-CD3 antibodies; expressing said recovered nucleic acid molecules in a host cell; and recovering antibodies from said host cell that bind Cadherin-17 and CD3 respectively.

As non-limiting examples, the disclosure provides for the preparation of the CD3 binding constructs wherein a host cell can be co-transfected with nucleic acids encoding the following pairings of SEQ ID NO: 38 and SEQ ID NO: 42; SEQ ID NO: 38 and SEQ ID NO: 43; SEQ ID NO: 38 and SEQ ID NO: 44; SEQ ID NO: 39 and SEQ ID NO: 42; SEQ ID NO: 39 and SEQ ID NO: 43; SEQ ID NO: 39 and SEQ ID NO: 44; SEQ ID NO: 40 and SEQ ID NO: 42; SEQ ID NO: 40 and SEQ ID NO: 43; SEQ ID NO: 40 and SEQ ID NO: 44; SEQ ID NO: 41 and SEQ ID NO: 42; SEQ ID NO: 41 and SEQ ID NO: 43; or SEQ ID NO: 41 and SEQ ID NO: 44.

As non-limiting examples, the disclosure provides for the CDH17 binding constructs wherein a host cell can be co-transfected with nucleic acids encoding the following pairings of SEQ ID NO: 2 and SEQ ID NO: 3; SEQ ID NO: 2 and SEQ ID NO: 4; SEQ ID NO: 5 and SEQ ID NO: 6; SEQ ID NO: 7 and SEQ ID NO: 9; or SEQ ID NO: 8 and SEQ ID NO: 9. Bispecific antibodies can be generated using controlled Fab arm exchanges or CH3 bispecific antibody mutations.

### Fc Region of Anti-CDH17, Anti-CD3, and CD3×CDH17 Bispecific Antibodies

The anti-CDH17, anti-CD3, and CD3×CDH17 bispecific antibodies disclosed herein may comprise a modified F_{c} region, wherein the modified F_{c} region comprises at least one amino acid modification relative to a native F_{c} region, for example, to extend the half-life of the bispecific antibody, enhance resistance of the bispecific antibody to proteolytic degradation, reduce effector functionality of the bispecific antibody, facilitate generation of the bispecific antibody by Fc heterodimerization, facilitate the multimerization of the bispecific antibody, and/or improve manufacturing and drug stability of the bispecific antibody.

In some embodiments, the Fc domain is altered to allow for silencing of the Fc domain to minimize effector function activity which can cause immune cell depletion and cytokine release syndrome.

In some embodiments, anti-CDH17, anti-CD3, and CD3×CDH17 bispecific antibodies as described herein are provided with a modified F_{c} region wherein a naturally occurring F_{c} region is modified to extend the half-life of the antibody when compared to the parental native antibody in a biological environment, for example, the serum half-life or a half-life measured by an in vitro assay. Exemplary mutations that may be made singularly or in combination are T250Q, M252Y, I253A, S254T, T256E, P257I, T307A, D376V, E380A, M428L, H433K, N434S, N434A, N434H, N434F, H435A and H435R mutations.

In certain embodiments, the extension of half-life can be realized by engineering the M252Y/S254T/T256E mutations in IgG1 F_{c} residue numbering according to the EU Index (Dall'Acqua, Kiener et al. 2006).

In certain embodiments, the extension of half-life can also be realized by engineering the M428L/N434S mutations in IgG₁ F_{c} (Zalevsky, Chamberlain et al. 2010).

In certain embodiments, the extension of half-life can also be realized by engineering the T250Q/M428L mutations in IgG₁ F_{c} (Hinton, Xiong et al. 2006).

In certain embodiments, the extension of half-life can also be realized by engineering the N434A mutations in IgG₁ F_{c} (Shields, Namenuk et al. 2001).

In certain embodiments, the extension of half-life can also be realized by engineering the T307A/E380A/N434A mutations in IgG₁ F_{c} (Petkova, Akilesh et al. 2006).

The effect F_{c} engineering on the extension of antibody half-life can be evaluated in PK studies in mice relative to antibodies with native IgG F_{c}.

In some embodiments, anti-CDH17, anti-CD3, and CD3×CDH17 bispecific antibodies as described herein are provided with a modified F_{c} region wherein a naturally occurring F_{c} region is modified to enhance the antibody resistance to proteolytic degradation by a protease that cleaves the wild-type antibody between or at residues 222-237 (EU numbering).

In certain embodiments, the resistance to proteolytic degradation can be realized by engineering E233P/L234A/L235A mutations in the hinge region with G236 deleted when compared to a parental native antibody, residue numbering according to the EU Index (Kinder, Greenplate et al. 2013).

In instances where effector functionality is not desired, the antibodies of the disclosure may further be engineered to introduce at least one mutation in the antibody F_{c} that reduces binding of the antibody to an activating Fey receptor (F_{c}γR) and/or reduces F_{c} effector functions such as C1q binding, complement dependent cytotoxicity (CDC), antibody-dependent cell-mediated cytotoxicity (ADCC) or phagocytosis (ADCP).

F_{c} positions that may be mutated to reduce binding of the antibody to the activating F_{c}γR and subsequently to reduce effector functions are those described for example in (Xu, Alegre et al. 2000) (Vafa, Gilliland et al. 2014) (Bolt, Routledge et al. 1993, Shields, Namenuk et al. 2001, Chu, Vostiar et al. 2008). Fc mutations with minimal ADCC, ADCP, CDC, Fc mediated cellular activation have been described also as sigma mutations for IgG1, IgG2 and IgG4 (Tam, McCarthy et al. 2017). Exemplary mutations that may be made singularly or in combination are K214T, E233P, L234V, L234A, deletion of G236, V234A, F234A, L235A, G237A, P238A, P238S, D265A, S267E, H268A, H268Q, Q268A, N297A, A327Q, P329A, D270A, Q295A, V309L, A327S, L328F, A330S and P331S mutations on IgG₁, IgG₂, IgG₃ or IgG₄.

Exemplary combination mutations that may be made to reduce ADCC are L234A/L235A on IgG₁, V234A/G237A/P238S/H268A/V309L/A330S/P331S on IgG₂, F234A/L235A on IgG₄, S228P/F234A/L235A on IgG₄, N297A on IgG₁, IgG₂, IgG₃ or IgG₄, V234A/G237A on IgG₂, K214T/E233P/L234V/L235A/G236 deleted/A327G/P331A/D365E/L358M on IgG₁, H268Q/V309L/A330S/P331S on IgG₂, S267E/L328F on IgG₁, L234F/L235E/D265A on IgG₁, L234A/L235A/G237A/P238S/H268A/A330S/P331S on IgG₁, S228P/F234A/L235A/G237A/P238S on IgG₄, and S228P/F234A/L235A/G236-deleted/G237A/P238S on IgG₄. Hybrid IgG_{2/4} F_{c} domains may also be used, such as F_{c} with residues 117-260 from IgG₂ and residues 261-447 from IgG₄.

In some embodiments, the CD3×CDH17 bispecific antibody is provided with a modified F_{c} region wherein a naturally occurring Fc region is modified to facilitate the generation of bispecific antibody by Fc heterodimerization.

In certain embodiments, the Fc heterodimerization can be realized by engineering F405L and K409R mutations on two parental antibodies and the generation of bispecific antibody in a process known as Fab arm exchange (Labrijn, Meesters et al. 2014).

In certain embodiments, the Fc heterodimerization can also be realized by Fc mutations to facilitate Knob-in-Hole strategy (see, e.g., Intl. Publ. No. WO 2006/028936). An amino acid with a small side chain (hole) is introduced into one Fc domain and an amino acid with a large side chain (knob) is introduced into the other Fc domain. After co-expression of the two heavy chains, a heterodimer is formed as a result of the preferential interaction of the heavy chain with a “hole” with the heavy chain with a “knob” (Ridgway, Presta et al. 1996). Exemplary Fc mutation pairs forming a knob and a hole are: T366Y/F405A, T366W/F405W, F405W/Y407A, T394W/Y407T, T394S/Y407A, T366W/T394S, F405W/T394S and T366W/T366S/L368A/Y407V.

In certain embodiments, the Fc heterodimerization can also be realized by Fc mutations to facilitate the electrostatically-matched interactions strategy (Gunasekaran, Pentony et al. 2010). Mutations can be engineered to generate positively charged residues at one Fc domain and negatively charged residues at the other Fc domain as described in US Patent Publ. No. US2010/0015133; US Patent Publ. No. US2009/0182127; US Patent Publ. No. US2010/028637 or US Patent Publ. No. US2011/0123532. Heavy chain heterodimerization can be formed by electrostatically matched interactions between two mutated F_{c}.

In some embodiments, the present CD3×CDH17 bispecific antibody is provided with a modified F_{c} region wherein a naturally occurring Fc region is modified to facilitate the multimerization of the antibody upon interaction with cell surface receptors, although such the bispecific antibody ordinarily exists as monomer in solution. The F_{c} mutations that facilitate antibody multimerization include, but are not limited to, E345R mutation, E430G mutation, E345R/E430G mutations, E345R/E430G/Y440R mutations as described in (Diebolder, Beurskens et al. 2014). Such mutations may also include, but are not limited to, T437R mutation, T437R/K248E mutations, and T437R/K338A mutations as described in (Zhang, Armstrong et al. 2017).

### Antibody Modifications

Antibodies further comprising conservative modifications are within the scope of the disclosure. “Conservative modifications” refer to amino acid modifications that do not significantly affect or alter the binding characteristics of the antibody containing the amino acid sequences. Conservative modifications include amino acid substitutions, additions and deletions. Conservative substitutions are those in which the amino acid is replaced with an amino acid residue having a similar side chain. The families of amino acid residues having similar side chains are well defined and include amino acids with acidic side chains (e.g., aspartic acid, glutamic acid), basic side chains (e.g., lysine, arginine, histidine), nonpolar side chains (e.g., alanine, valine, leucine, isoleucine, proline, phenylalanine, methionine), uncharged polar side chains (e.g., glycine, asparagine, glutamine, cysteine, serine, threonine, tyrosine, tryptophan), aromatic side chains (e.g., phenylalanine, tryptophan, histidine, tyrosine), aliphatic side chains (e.g., glycine, alanine, valine, leucine, isoleucine, serine, threonine), amide (e.g., asparagine, glutamine), beta-branched side chains (e.g., threonine, valine, isoleucine) and sulfur-containing side chains (cysteine, methionine). Furthermore, any native residue in the polypeptide may also be substituted with alanine, as has been previously described for alanine scanning mutagenesis. Amino acid substitutions to the antibodies of the disclosure may be made by known methods for example by PCR mutagenesis (U.S. Pat. No. 4,683,195). Alternatively, libraries of variants may be generated for example using random (NNK) or non-random codons, for example DVK codons, which encode 11 amino acids (Ala, Cys, Asp, Glu, Gly, Lys, Asn, Arg, Ser, Tyr, Trp). The resulting antibody variants may be tested for their characteristics using assays described herein.

The antibodies of this disclosure may be post-translationally modified by processes such as glycosylation, isomerization, deglycosylation or non-naturally occurring covalent modification such as the addition of polyethylene glycol moieties (pegylation) and lipidation. Such modifications may occur in vivo or in vitro. For example, the antibodies of the disclosure may be conjugated to polyethylene glycol (PEGylated) to improve their pharmacokinetic profiles. Conjugation may be carried out by techniques known to those skilled in the art. Conjugation of therapeutic antibodies with PEG has been shown to enhance pharmacodynamics while not interfering with function.

Antibodies of this disclosure may be modified to improve stability, selectivity, cross-reactivity, affinity, immunogenicity or other desirable biological or biophysical property are within the scope of the disclosure. Stability of an antibody is influenced by a number of factors, including (1) core packing of individual domains that affects their intrinsic stability, (2) protein/protein interface interactions that have impact upon the HC and LC pairing, (3) burial of polar and charged residues, (4) H-bonding network for polar and charged residues; and (5) surface charge and polar residue distribution among other intra- and inter-molecular forces (Worn and Pluckthun 2001). Potential structure destabilizing residues may be identified based upon the crystal structure of the antibody or by molecular modelling in certain cases, and the effect of the residues on antibody stability may be tested by generating and evaluating variants harboring mutations in the identified residues. One of the ways to increase antibody stability is to raise the thermal transition midpoint (Tₘ) as measured by differential scanning calorimetry (DSC). In general, the protein Tₘ is correlated with its stability and inversely correlated with its susceptibility to unfolding and denaturation in solution and the degradation processes that depend on the tendency of the protein to unfold. A number of studies have found correlation between the ranking of the physical stability of formulations measured as thermal stability by DSC and physical stability measured by other methods. Formulation studies suggest that a Fab Tₘ has implication for long-term physical stability of a corresponding mAb.

Antibodies of this disclosure may have amino acid substitutions in the F_{c} region that improve manufacturing and drug stability. An example for IgG₁ is H224S (or H224Q) in the hinge 221-DKTHTC-226 (EU numbering) which blocks radically induced cleavage; and for IgG₄, the S228P mutation blocks half-antibody exchange.

### Shielding or Masking Domains

As non-limiting examples, the disclosure provides a bispecific antibody comprising a shielding domain (also referred to as masking domain, mask, or cap) selected from the shielding domain amino acid sequences set forth as SEQ ID NOs: 47-50 (e.g., for the CDH17 mAb arm) and SEQ ID NO: 51 (for the CD3 mAb arm) in Table 5.

TABLE 5

| | | |
|---|---|---|
| 47 | IgG1 hinge | EPKSCDKTHTCPPCP |
| 48 | Fibronectin peptide | DQCIVDDITYNVQDTFHKKHEEGHMLNCTCFGQGRGRW |
| | | KCDPVDQCQDSETGTFYQIGDSWEKYVHGVRYQCYCYG |
| | | RGIGEWHCQPLQTYPSS |
| 49 | Collagen peptide 1 | GQRGVVGLPGQRGERGFPGLPGY |
| 50 | Collagen peptide 2 | GQRGVVGLPGQRGER |
| 51 | Anti-CD3 antibody shield | QDGNEEMGG |

### Protease-Cleavable Linkers

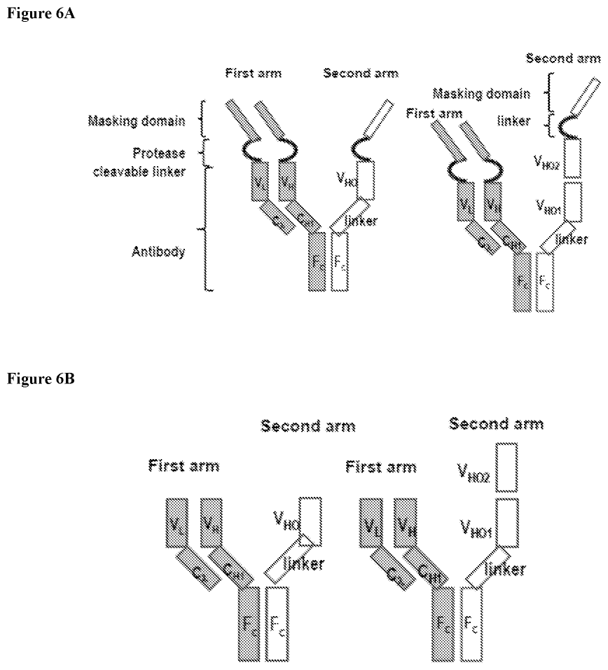

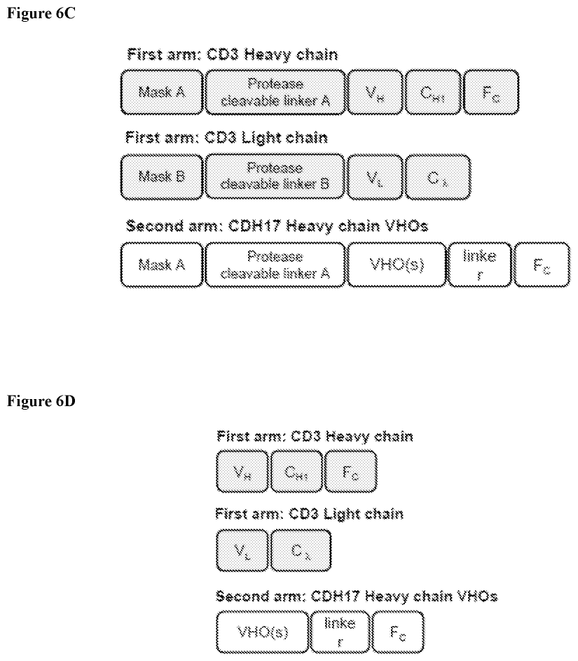

The protease-cleavable linker comprises a peptide substrate cleavable by a protease linking the shielding domain to the antibody heavy or light chains. The protease-cleavable linker comprises one or more protease substrate sequence and optional linker spacer sequences (see e.g., FIG. 6). In some embodiments, the shielding sequences exist as pairs of sequences that can be fused to the heavy chain and light chain. For example, for each of the two Fab arm domains of a bispecific antibody, a shielding sequence is fused to the N-terminus of the antibody heavy chain via one protease-cleavable linker and the complement sequence is fused to the N-terminus of the antibody light chain via another protease-cleavable linker.

Many disease tissues, including tumor microenvironment and inflammation site, are abundant with various types of proteases whose overexpression correlate with the disease progression. In disease tissues, the protease-cleavable linker sequences of the shielded antibody are recognized by appropriate type of protease and the shields can be released from the antibody chains. For example, in one binding arm of a shielded bispecific antibody, the protease may cleave both of the two protease-cleavable linkers or one of the two protease-cleavable linker sequences, so the shielding domain is inactive. In either case, the shielding domain would not be able to interfere or block the binding of the Fab arm to its target antigen. As a result, the shielded antibody is converted into an active antibody to bind and exert its functional activity to its target (FIG. 7).

In some embodiments, the protease-cleavable linker sequences linking the two shielding domains to the two Fab domains in a shielded antibody comprise the same sequences to be cleaved by the same type of protease.

In some embodiments, the protease-cleavable linker sequences linking the two masking domains and the two Fab domains in a shielded antibody comprise different sequences with substrate sequences cleaved by different types of proteases.

Among the family of matrix metalloproteinases (MMPs), MMP2 and MMP9 are up regulated in many types of cancers, including breast, colorectal and lung cancers. Besides, the expression and activity of MMP2 and MMP9 also correlates to the progression of many autoimmune disorders and inflammatory diseases, including rheumatoid arthritis, psoriasis, multiple sclerosis, chronic obstructed pulmonary disease, inflammatory bowel disease and osteoporosis (Lin, Lu et al. 2020). The disclosure provides for the protease-cleavable linker sequence comprising substrate peptide sequence cleaved by MMP2 and MMP9. As non-limiting examples, the disclosure provides for the MMP2 and MMP9 cleavable substrate peptide sequences set forth as SEQ ID NOs: 52-56. As non-limiting examples, the disclosure provides for the MMP3 cleavable substrate peptide sequences set forth as SEQ ID NO: 57.

The urokinase plasminogen activator (uPA) has been reported to be overexpressed in many types of cancer, especially the breast cancer (Banys-Paluchowski, Witzel et al. 2019). uPA is a serine protease that can catalyze the conversion of plasminogen to plasmin which can degrade the basement membrane or extracellular matrix. The matrix degradation can facilitate tumor cells migration and invasion into the surrounding tissue. The disclosure provides for the protease-cleavable linker sequence comprising substrate peptide sequence cleaved by uPA. As non-limiting examples, the disclosure provides for the uPA-cleavable substrate peptide sequence set forth as SEQ ID NOs: 58 and 59 in Table 6.

TABLE 6

| | | |
|---|---|---|
| 52 | Substrate sequence for MMP2 and MMP9 | GPLGVR |
| 53 | Substrate sequence for MMP2 and MMP9 | PLGLAR |
| 54 | Substrate sequence for MMP2 and MMP9 | PLGLAG |
| 55 | Substrate sequence for MMP2 and MMP9 | IPVSLRSG |
| 56 | Substrate sequence for MMP2 and MMP9 | GPLGMLSQ |
| 57 | Substrate sequence for MMP3 | RPKPVEVWRK |
| 58 | Substrate sequence for uPA | LSGRSDNH |
| 59 | Substrate sequence for uPA | TGRGPSWV |

The protease-cleavable linker of the present disclosure can include one or more linker peptides interposed between, e.g., shielding sequence and protease substrate peptide sequence, and/or between protease substrate peptide sequence and antibody Fabs.

Suitable linkers (also referred to as “spacers”) can be readily selected and can be of any of a number of suitable lengths, such as from 1 amino acid to 30 amino acids (e.g., any specific integer between 1 and 30, or from 1 amino acid (e.g., Gly) to about 20 amino acids, from 2-15, 3-12, 4-10, 5-9, 6-8, or 7-8 amino acids).

Exemplary linkers include glycine polymers (G) n, glycine-serine polymers (including, for example, (GS)ₙ, (GSGGS)ₙ and (GGGS)ₙ, where n is an integer of at least one, e.g., 1, 2, 3, 4, 5, 6, 7, 8, 9, 10, 11, 12, 13, 14, 15, 16, 17, 18, 19, 20), glycine-alanine polymers, alanine-serine polymers, alanine-proline, immunoglobulin isotype and subtype hinge that can comprise IgG₁, IgG₂, IgG₃, IgG₄, IgA, IgE, IgM, and other flexible linkers known in the art. Both Gly and Ser are relatively unstructured, and therefore can serve as a neutral tether between components.

In certain embodiments, the linker is a Glycine polymer. Glycine accesses significantly more phi-psi space than even alanine and is much less restricted than residues with longer side chains (Scheraga 2008). Exemplary linkers can comprise amino acid sequences including, but not limited to: GGS; GGSG; GGSGG; GGGGS; GSGSG; GSGGG; GGGSG; GSSSG, and the like.

In certain embodiments, the linker is an Alanine-Proline polymer. Exemplary linkers can comprise amino acid sequences including, but not limited to (AP)ₙ, where n is an integer of at least one, e.g., 1, 2, 3, 4, 5, 6, 7, 8, 9, 10, 11, 12, 13, 14, 15, 16, 17, 18, 19, 20).

In certain embodiments, the linker is a rigid linker (Chen, Zaro et al. 2013). Exemplary rigid linkers can comprise amino acid sequences including, but not limited to, proline-rich sequence, (XP)ₙ, with X designating any amino acid, preferably Ala, Lys, or Glu, where n is an integer of at least one, e.g., 1, 2, 3, 4, 5, 6, 7, 8, 9, 10, 11, 12, 13, 14, 15, 16, 17, 18, 19, 20). Exemplary rigid linkers can also comprise amino acid sequences including, but not limited to, alpha helix-forming linkers with the sequence of (EAAAK)ₙ, where n is an integer of at least one, e.g., 1, 2, 3, 4, 5, 6, 7, 8, 9, 10, 11, 12, 13, 14, 15, 16, 17, 18, 19, 20).

### Expression and Purification of Antibodies

The antibodies of the disclosure can be encoded by one or more nucleic acids for protein expression. For example, a shielded CD3×CDH17 bispecific antibody of the present disclosure can be encoded by a single nucleic acid (e.g., a single nucleic acid comprising nucleotide sequences that encode the light and heavy chain polypeptides of the shielded antibody), or by two or more separate nucleic acids, each of which encodes a different part of the shielded parental antibody.

The recombinant DNA is prepared by DNA recombination techniques and then transfected into mammalian cells, the corresponding anti-CD3 and anti-CDH17 antibodies are expressed, purified, identified, and/or screened. A bispecific antibody can be generated from anti-CD3 and anti-CDH17 antibodies using controlled Fab arm exchange or other bispecific antibody generation process to produce a bispecific antibody that shows biological effects of simultaneous binding to CDH17 and CD3. The bispecific antibody affinity and blocking efficiency are identified through in vitro experiments.

The nucleic acids described herein can be inserted into vectors, e.g., nucleic acid expression vectors and/or targeting vectors. Such vectors can be used in various ways, e.g., for the expression of a pro-antibody (shielded antibody) with a masking domain described herein in a cell or transgenic animal. Vectors are typically selected to be functional in the host cell in which the vector will be used. A nucleic acid molecule encoding an antibody, e.g., a pro-antibody with a masking domain described herein may be amplified/expressed in prokaryotic, yeast, insect (baculovirus systems) and/or eukaryotic host cells. Selection of the host cell will depend in part on whether the antibodies disclosed herein, such as a shielded CD3×CDH17 bispecific antibody described herein, is to be post-translationally modified (e.g., glycosylated and/or phosphorylated). If so, yeast, insect, or mammalian host cells are preferable. Expression vectors typically contain one or more of the following components: a promoter, one or more enhancer sequences, an origin of replication, a transcriptional termination sequence, a complete intron sequence containing a donor and acceptor splice site, a leader sequence for secretion, a ribosome binding site, a polyadenylation sequence, a polylinker region for inserting the nucleic acid encoding the polypeptide to be expressed, and a selectable marker element.

In some embodiments, a leader or signal sequence is engineered at the N-terminus of the antibodies, e.g., a shielded CD3×CDH17 bispecific antibody described herein to guide its secretion. The secretion of the shielded CD3×CDH17 bispecific antibody from a host cell will result in the removal of the signal peptide from the antibody. Thus, the mature shielded CD3×CDH17 bispecific antibody will lack any leader or signal sequence. In some embodiments, such as where glycosylation is desired in a eukaryotic host cell expression system, one may manipulate the various presequences to improve glycosylation or yield. For example, one may alter the peptidase cleavage site of a signal peptide, or add prosequences, which also may affect glycosylation.

The disclosure further provides a cell (e.g., an isolated or purified cell) comprising a nucleic acid or vector of the disclosure. The cell can be any type of cell capable of being transformed with the nucleic acid or vector of the disclosure so as to produce a polypeptide encoded thereby. For example, to express the shielded CD3×CDH17 bispecific antibody described herein, DNAs encoding partial or full-length light and heavy chains, obtained as described above, are inserted into expression vectors such that the genes are operatively linked to transcriptional and translational control sequences.

Methods of introducing nucleic acids and vectors into isolated cells and the culture and selection of transformed host cells in vitro are known in the art and include the use of calcium chloride-mediated transformation, transduction, conjugation, triparental mating, DEAE, dextran-mediated transfection, infection, membrane fusion with liposomes, high velocity bombardment with DNA-coated microprojectiles, direct microinjection into single cells, and electroporation.

After introducing the nucleic acid or vector of the disclosure into a host cell, the cell is cultured under conditions suitable for expression of the encoded sequence. The antibody, antigen binding fragment, or portion of the antibody then can be isolated from the cell.

In certain embodiments, two or more vectors that together encode the shielded CD3×CDH17 bispecific antibody described herein, can be introduced into a host cell.

Purification of an antibody, e.g., a shielded CD3×CDH17 bispecific antibody described herein, which is secreted into the cell media, can be accomplished using a variety of techniques including affinity, immunoaffinity or ion exchange chromatography, molecular sieve chromatography, preparative gel electrophoresis or isoelectric focusing, chromatofocusing, and high-pressure liquid chromatography. For example, antibodies comprising a F_{c} region may be purified by affinity chromatography with Protein A, which selectively binds the F_{c} region.

Modified forms of the antibodies, such as a shielded CD3×CDH17 bispecific antibody may be prepared with affinity tags, such as hexahistidine or other small peptide such as FLAG (Eastman Kodak Co., New Haven, Conn.) or Myc (Invitrogen) at either its carboxyl or amino terminus and purified by a one-step affinity column. For example, Poly histidine binds with great affinity and specificity to nickel, thus an affinity column of nickel (such as the Qiagen® nickel columns) can be used for purification of Poly histidine-tagged selective binding agents. In some instances, more than one purification step may be employed.

### Effects of Shielded CD3×CDH17 Bispecific Antibody on Binding and Functional Activity

A shielded CD3×CDH17 bispecific antibody disclosed herein can inhibit or block the capability of the Fab arms to bind to the respective antigens, CD3 and CDH17. The masking domains may reduce the maximum binding capacity of the shielded bispecific antibody in binding to the respective antigens. The masking domains may also reduce the binding affinity of the shielded bispecific antibody in binding to the respective antigens.

When the masking domains are cleaved off by protease, the shielded antibody is converted to an active bispecific antibody with the restoration of the capability of the antibody in binding to its antigen. The removal of masking domains from the shielded bispecific antibody can be realized by in vitro protease cutting assay using recombinant or purified protease. The removal of the masking domains from the shielded bispecific antibody can also be realized in vivo by proteases overexpressed in disease site. The removal of the masking domains can be assessed by comparing the molecular weight of heavy chain and light chain of shielded antibodies with the masking domains to the active antibody without the masking domains by SDS-PAGE, IEX, or HIC analyses.

In vitro and cell-based assays are well described in the art for use in determining the pro-antibody (shielded bispecific antibody), active antibody, and converted antibody after protease cleavage in binding to its antigen. For example, the binding of an antibody may be determined by ELISA by immobilizing a recombinant or purified antigen, sequestering antibody with the immobilized antigen and determining the amount of bound antibody. This can also be performed using a Biacore® instrument for kinetic analysis of binding interactions. For cell-based binding assay, the binding of an antibody may be determined by flow cytometry by incubating the antibody with cells expressing antigens on cell surface and determining the amount of antibody bound to cell surface antigens.

### Pharmaceutical Compositions

The antibodies such as shielded CD3×CDH17 bispecific antibodies for use according to the present disclosure can be formulated in compositions, especially pharmaceutical compositions, for use in the methods described herein. Such compositions comprise a therapeutically or prophylactically effective amount of an antibody, e.g., a bispecific antibody described in this disclosure and a suitable carrier, e.g., a pharmaceutically acceptable agent. Typically, the antibody described in this disclosure is sufficiently purified for administration to an animal before formulation in a pharmaceutical composition.

In some embodiment, the present disclosure provides a composition comprising an anti-CDH17 antibody or an antigen binding portion thereof. In some embodiment, the present disclosure provides a pharmaceutical composition comprising an anti-CDH17 antibody or an antigen binding portion thereof and a pharmaceutically acceptable carrier.

In some embodiment, the present disclosure provides a composition comprising an anti-CD3 antibody or an antigen binding portion thereof. In some embodiment, the present disclosure provides a pharmaceutical composition comprising an anti-CD3 antibody or an antigen binding portion thereof and a pharmaceutically acceptable carrier.

In some embodiment, the present disclosure provides a composition comprising a shielded or non-shielded CD3×CDH17 bispecific antibody. In some embodiment, the present disclosure provides a pharmaceutical composition comprising a shielded or non-shielded CD3×CDH17 bispecific antibody and a pharmaceutically acceptable carrier.

Pharmaceutically acceptable agents include carriers, excipients, diluents, antioxidants, preservatives, coloring, flavoring and diluting agents, emulsifying agents, suspending agents, solvents, fillers, bulking agents, buffers, delivery vehicles, tonicity agents, cosolvents, wetting agents, complexing agents, buffering agents, antimicrobials, and surfactants.

The composition can be in liquid form or in a lyophilized or freeze-dried form and may include one or more lyoprotectants, excipients, surfactants, high molecular weight structural additives and/or bulking agents.

In some embodiments, the composition comprises a shielded CD3×CDH17 bispecific antibody, or an anti-Cadherin-17 antibody or an antigen-binding portion thereof, or an anti-CD3 antibody or an antigen binding portion thereof, and at least one buffer, at least one stabilizer, and/or at least one surfactant.

In some embodiments, the composition disclosed herein is liquid. In some embodiments, the composition is formulated for subcutaneous injection. In some embodiments, the composition is sterile. In some embodiments, the composition further comprises histidine HCl, trehalose, methionine and/or polysorbate.

Compositions can be suitable for parenteral administration. Exemplary compositions are suitable for injection or infusion into an animal by any route available to the skilled worker, such as intraarticular, subcutaneous, intravenous, intramuscular, intraperitoneal, intracerebral (intraparenchymal), intracerebroventricular, intramuscular, intraocular, intraarterial, intralesional, intrarectal, transdermal, oral, and inhaled routes.

Pharmaceutical compositions described herein can be formulated for controlled or sustained delivery in a manner that provides local concentration of the product (e.g., bolus, depot effect) sustained release and/or increased stability or half-life in a particular local environment.

In some embodiments, the CD3×CDH17 bispecific antibody or an anti-Cadherin-17 antibody or an antigen-binding portion thereof, or an anti-CD3 antibody or an antigen binding portion thereof, can be present in a pharmaceutical composition at a concentration of 1 mg/mL to 250 mg/mL, 10 mg/mL to 250 mg/mL, 1 mg/mL to 100 mg/mL, 2 mg/mL to 50 mg/mL, and 2 mg/mL to 40 mg/mL.

### Methods of Treatment and Use

In some embodiments, the present disclosure provides methods for treating or preventing a disease or disorder in a subject in need thereof comprising administering an effective amount of a shielded CD3×CDH17 bispecific antibody or an anti-Cadherin-17 antibody or an antigen-binding portion thereof disclosed herein to the subject. The diseases or disorder is selected from CDH17 mediated diseases or disorders, e.g., gastric (small intestine neuroendocrine tumors (SINETs)), lung, pancreatic (e.g., pancreatic neuroendocrine tumors (PanNETs)), colorectal, and hepatocellular carcinoma, and/or other cancers.

In some embodiments, the disease or disorder disclosed herein is conventional, preferably, lung cancer, breast cancer, pancreatic cancer, or gastric cancer.

In some embodiments, the present disclosure provides a shielded CD3×CDH17 bispecific antibody or an anti-Cadherin-17 antibody or an antigen-binding portion thereof disclosed herein for use in treating or preventing a disease or disorder selected from CDH17 mediated diseases or disorders.

In some embodiments, the present disclosure provides the use of a shielded CD3×CDH17 bispecific antibody or an anti-Cadherin-17 antibody or an antigen-binding portion thereof disclosed herein for the manufacture of a medicament for use in treating or preventing a disease or disorder selected from CDH17 mediated diseases or disorders.

In some embodiments, the present disclosure provides a shielded CD3×CDH17 bispecific antibody or an anti-Cadherin-17 antibody or an antigen-binding portion thereof described herein for use in treating or preventing gastric, lung, pancreatic, colorectal, and/or other cancers. In contrast to corresponding therapeutic antibodies, the shielded CD3×CDH17 bispecific antibody may have comparable efficacy in treating these diseases due to the conversion of the shielded antibody to active antibody specifically in disease sites by the removal of the shielding domains by proteases overexpressed in disease sites. However, the shielded antibody may have reduced systematic toxicity due to the masking of the antibody activity by the shielding domains in normal tissues that lack sufficient amounts of proteases needed to cleave off the masking domains. The shielded bispecific antibody described herein may be efficacious as the corresponding therapeutic antibody in treating diseases but with much improved safety profile. Due to the improved safety profile, increased levels of dosing comprising the shielded bispecific antibodies may be administered to patients with improved treatment efficacy.

In some embodiments, the disclosure also provides for a method of treating cancer, in a subject, comprising administering a therapeutically effective amount of a shielded CD3×CDH17 bispecific antibody. The disclosure also provides for use of the shielded bispecific provided herein in a method of treating cancer; and for use of the shielded CD3×CDH17 bispecific antibodies or an anti-Cadherin-17 antibodies or antigen-binding portions thereof provided herein in the manufacture of a medicament for use in cancer. Exemplary cancers include, but are not limited to multiple myeloma, non-small cell lung cancer, acute myeloid leukemia, female breast cancer, pancreatic cancer, colorectal cancer and peritoneum cancer.

Some embodiments provide for a bispecific antibody that can effectively inhibit CDH17 receptor association with integrins and downstream signaling.

In some embodiments, a bispecific antibody or an anti-CDH 17 antibody disclosed herein can be used a combination with chemotherapy. For example, a combination regimen for treating cancer can use higher doses of chemotherapy and a CD3×CDH 17 bispecific antibody or anti-CDH 17 to determine the best synergistic partners.

In some embodiments, the present disclosure provides a conjugate, which comprises an anti-Cadherin-17 antibody of the disclosure conjugated to another therapeutic agent. In some embodiments of the disclosure, the therapeutic agent comprises a cytotoxin or a radioactive isotope.

All combinations of the various elements described herein are within the scope of the disclosure unless otherwise indicated herein or otherwise clearly contradicted by context.

This disclosure will be better understood from the following Experimental Details. However, one skilled in the art will readily appreciate that the specific methods and results discussed are merely illustrative of the disclosure as described more fully in the claims that follow thereafter.

### Example 1: Expression and Purification of Anti-CDH17 and Anti-CD3 Antibodies

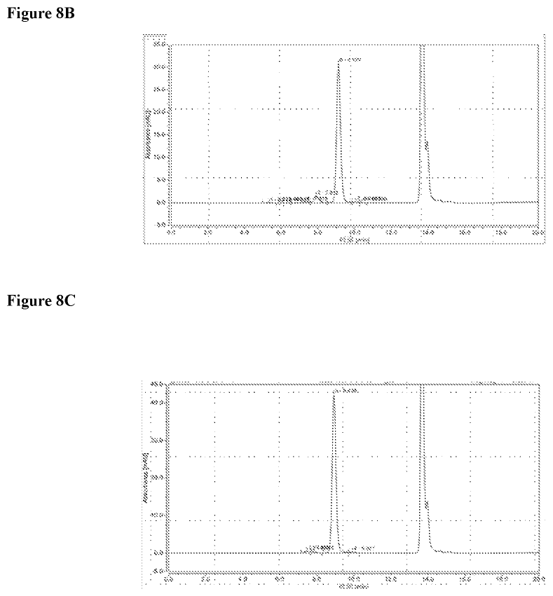

Heavy chain and light chain constructs expressing anti-CD3 and anti-CDH17 mAbs were prepared. Plasmids encoding the respective heavy chains and light chains of these anti-CD3 and anti-CDH17 masked or unmasked antibodies were co-transfected into Expi293F cells following the transfection kit instructions (Thermo Scientific). Cells were spun down five days post transfection, and the supernatant were passed through a 0.2 μm filter. Purifications of the expressed antibody supernatants were carried out by affinity chromatography over protein A agarose columns (GE Healthcare Life Sciences). The purified antibodies were buffer exchanged into DPBS, pH7.2 by dialysis, and protein concentrations were determined by UV absorbance at 280 nm. The purity of the protein A purified anti-CDH17 antibodies were determined via reducing SDS-PAGE. FIG. 8A shows reduced SDS-PAGE of some example CDH17 mAbs. The proteins had typical human IgG heavy chain and light chain band patterns on a reduced SDS-PAGE gel. The monomeric form of the CDH17 antibodies were confirmed by running HPLC with a size-exclusion column. Two examples of anti-CDH17 antibodies are shown (FIG. 8B). Both antibodies had typical IgG1 SEC profiles.

### Example 2: Characterization of Bispecific Antibodies

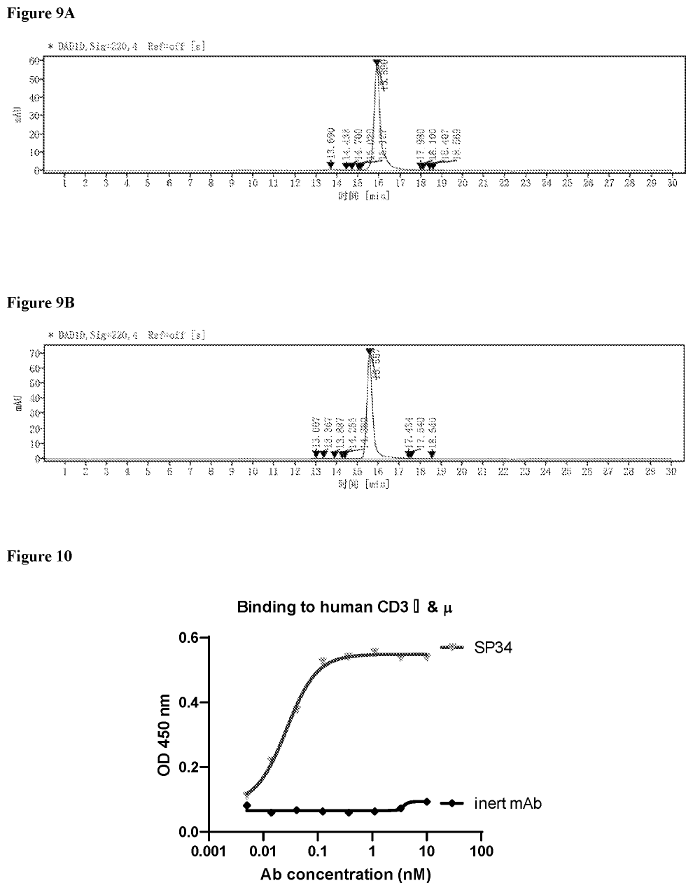

Bispecific CD3×CDH17 antibodies were prepared using anti-CD3 and anti-CDH17 parental antibodies. The bispecific antibodies CD3×CDH17 v1 and CD3×CDH17 v2 were prepared using controlled Fab arm exchange technology. After dialysis, the monomeric state of these bispecific antibodies were confirmed using HPLC SEC. The results in FIGS. 9A and 9B confirmed that the bispecific antibodies had similar monomeric state as a typical human IgG1 molecule.

### Example 3: Binding of CD3 by Anti-CD3 mAbs

ELISA-based binding assay was employed to evaluate the binding to CD3 by anti-CD3 antibodies. In this assay, 100 μL of 1 mg/mL recombinant human CD3 (R&D systems) was coated on an ELISA plate. Increasing concentrations of masked and reference mAbs were applied on the plate and their binding to the recombinant human proteins were detected by HRP-conjugated anti-human secondary antibody. FIG. 10 demonstrated that the SP34 anti-CD3 antibody (SEQ ID NO: 45 and SEQ ID NO: 46) had potent binding to CD3 via ELISA binding.

### Example 4: Binding of CDH17 by Anti-CDH17 Antibodies

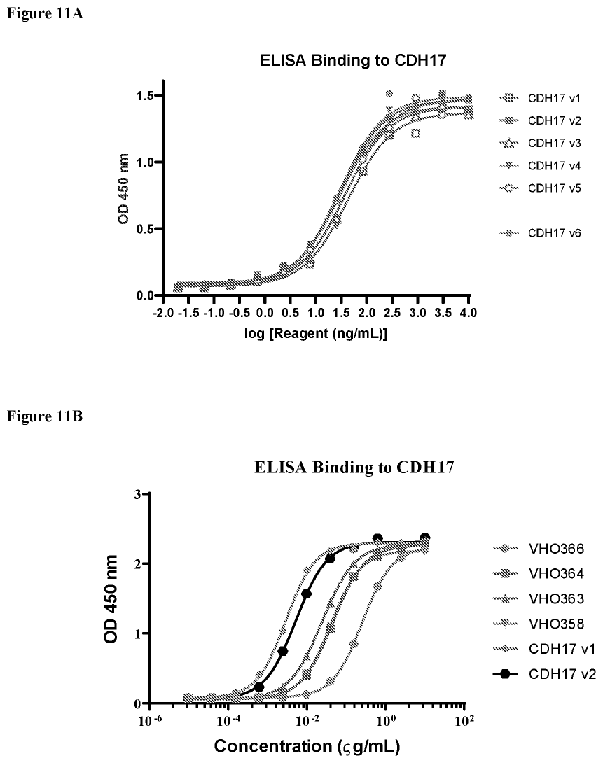

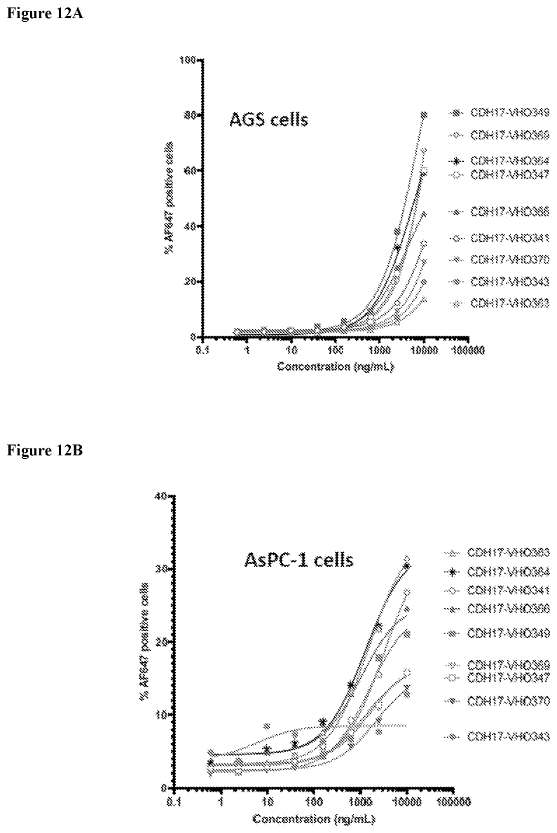

ELISA-based binding assay was employed to evaluate the binding to CDH17 by anti-CDH17 antibodies. In this assay, 100 μL of 1 mg/mL recombinant human CDH17 (R&D systems) was coated on an ELISA plate. Increasing concentrations of masked and reference mAbs were applied on the plate and their binding to the recombinant human proteins were detected by HRP-conjugated anti-human secondary antibody. FIG. 11 shows an evaluation of anti-CDH17 mAbs binding to CDH17. The expressed anti-CDH17 mAbs had potent binding to CDH17. The anti-CDH17 were tested for binding to AGS gastric cancer cells and AsPC-1 pancreatic cancer cells. FIGS. 12-14 demonstrated that the anti-CDH17 VHOs (variable heavy only single domain antibodies) had a range of binding to the CDH17 bearing AGS gastric cancer cells and AsPC-1 pancreatic cancer cells.

In FIG. 12, VHO v349 represented a monoclonal antibody comprising a heavy chain variable region as set forth in SEQ ID NO: 10. VHO v369 represented a monoclonal antibody comprising a heavy chain variable region as set forth in SEQ ID NO: 11. VHO v364 represented a monoclonal antibody comprising a heavy chain variable region as set forth in SEQ ID NO: 14. VHO v347 represented a monoclonal antibody comprising a heavy chain variable region as set forth in SEQ ID NO: 12. VHO v346 represented a monoclonal antibody comprising a heavy chain variable region as set forth in SEQ ID NO: 13. VHO v341 represented a monoclonal antibody comprising a heavy chain variable region as set forth in SEQ ID NO: 16. VHO v370 represented a monoclonal antibody comprising a heavy chain variable region as set forth in SEQ ID NO: 33. VHO v343 represented a monoclonal antibody comprising a heavy chain variable region as set forth in SEQ ID NO: 27. VHO v363 represented a monoclonal antibody comprising a heavy chain variable region as set forth in SEQ ID NO: 15. CDH17-VHO364 was used as a reference binder for these CDH17 ELISA binding experiments.

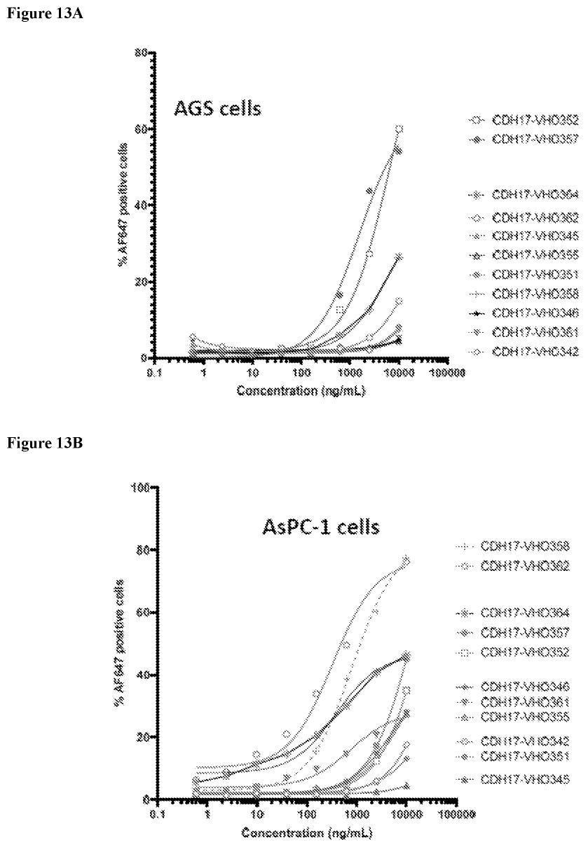

FIG. 13A demonstrated that the anti-CDH17 single domain Abs bound to CDH17 expressing AGS cells and FIG. 13B demonstrated binding to CDH17 expressing AsPC-1 pancreatic cancer cells by flow cytometry. VHO v352 represented a monoclonal antibody comprising a heavy chain variable region as set forth in SEQ ID NO: 18. VHO v357 represented a monoclonal antibody comprising a heavy chain variable region as set forth in SEQ ID NO: 17. VHO v362 represented a monoclonal antibody comprising a heavy chain variable region as set forth in SEQ ID NO: 20. VHO v351 represented a monoclonal antibody comprising a heavy chain variable region as set forth in SEQ ID NO: 30. VHO v358 represented a monoclonal antibody comprising a heavy chain variable region as set forth in SEQ ID NO: 19. VHO v361 represented a monoclonal antibody comprising a heavy chain variable region as set forth in SEQ ID NO: 21. VHO v342 represented a monoclonal antibody comprising a heavy chain variable region as set forth in SEQ ID NO: 26. VHO v345 represented a monoclonal antibody comprising a heavy chain variable region as set forth in SEQ ID NO: 28. VHO v355 represented a monoclonal antibody comprising a heavy chain variable region as set forth in SEQ ID NO: 31. VHO v346 represented a monoclonal antibody comprising a heavy chain variable region as set forth in SEQ ID NO: 29. CDH17-VHO364 was used as a reference binder for these CDH17 ELISA binding experiments.

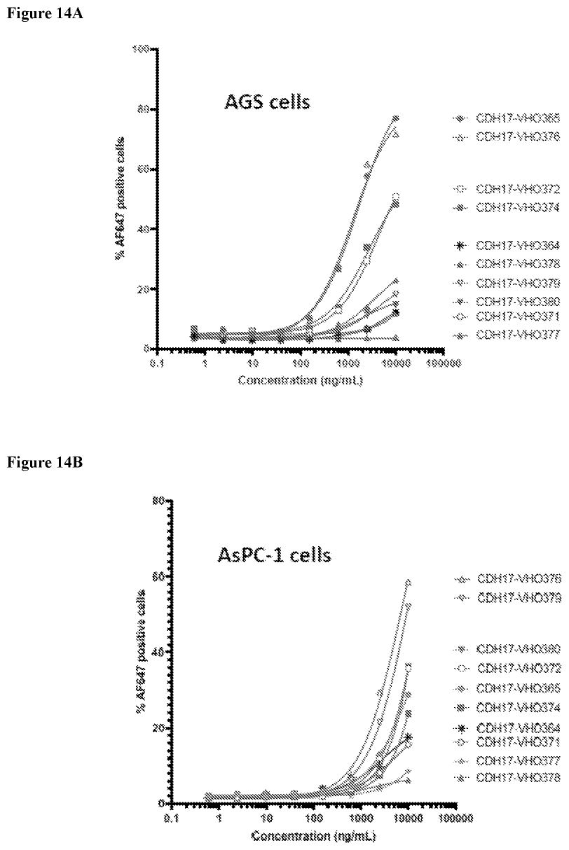

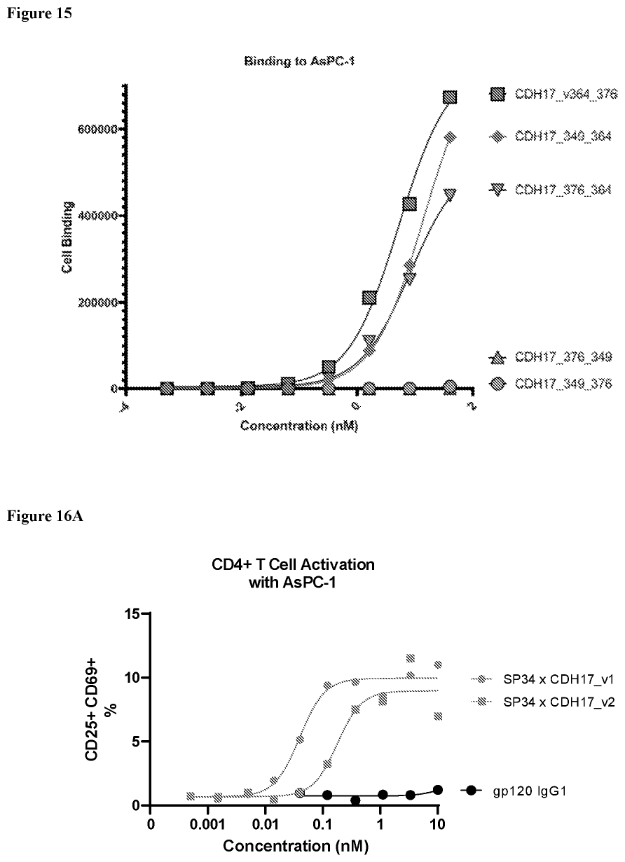

FIG. 14 demonstrated that the anti-CDH17 single domain Abs bound to (A) CDH17 expressing AGS cells and (B) CDH17 expressing AsPC-1 pancreatic cancer cells by flow cytometry. VHO v365 represented a monoclonal antibody comprising a heavy chain variable region as set forth in SEQ ID NO: 21. VHO v376 represented a monoclonal antibody comprising a heavy chain variable region as set forth in SEQ ID NO: 145. VHO v372 represented a monoclonal antibody comprising a heavy chain variable region as set forth in SEQ ID NO: 23. VHO v374 represented a monoclonal antibody comprising a heavy chain variable region as set forth in SEQ ID NO: 35. VHO v378 represented a monoclonal antibody comprising a heavy chain variable region as set forth in SEQ ID NO: 37. VHO v379 represented a monoclonal antibody comprising a heavy chain variable region as set forth in SEQ ID NO: 25. VHO v380 represented a monoclonal antibody comprising a heavy chain variable region as set forth in SEQ ID NO: 37. VHO v371 represented a monoclonal antibody comprising a heavy chain variable region as set forth in SEQ ID NO: 34. VHO v377 represented a monoclonal antibody comprising a heavy chain variable region as set forth in SEQ ID NO: 36. CDH17-VHO364 was used as a reference binder for these CDH17 ELISA binding experiments.

The binding of the anti-CDH17 antibodies was detected and quantified using standard flow cytometric methods. These data demonstrate that the tested anti-CDH17 antibodies are capable of binding to the human CDH17 protein as it is natively presented on the surface of the intended target cancer cells in addition to their demonstrated ability to bind to the isolated recombinant protein by ELISA.

### Example 5: Tandem Anti-CDH17 Binding to AsPC-1 Cells

Several tandem VHO based anti-CDH17 antibodies were prepared. CDH17_v364_376 represented a monoclonal antibody comprising an amino-terminal heavy chain VHO v364 was fused to VHO v376 via a (GGGGS)₄ linker on a human IgG1 Fc. CDH17_v349_364 represented a monoclonal antibody comprising an amino terminal heavy chain VHO v349 was fused to VHO v364 via a (GGGGS)₄ linker on a human IgG1 Fc. CDH17_v376_364 represented a monoclonal antibody comprising an amino terminal heavy chain VHO v376 was fused to VHO v364 via a (GGGGS)₄ linker on a human IgG1 Fc. CDH17_v376_349 represented a monoclonal antibody comprising an amino terminal heavy chain VHO v376 was fused to VHO v349 via a (GGGGS)₄ linker on a human IgG1 Fc. CDH17_v349_376 represented a monoclonal antibody comprising an amino terminal heavy chain VHO v349 was fused to VHO v376 via a (GGGGS)₄ linker on a human IgG1 Fc. The CDH17_v364_376, CDH17_v349_364, and CDH17_v376_364 molecules could bind to AsPC-1 pancreatic cancer cells. However, the CDH17_v376_349 and CDH17_v349 376 molecules could bind to AsPC-1 pancreatic cancer cells. Thus selection of the CDH17 single domain orientation and components were important for binding to CDH17 containing cells such as AsPC-1.

### Example 6: Activation of CD3+ T Cells in the Presence of Pancreatic Cancer Cell Line AsPC-1

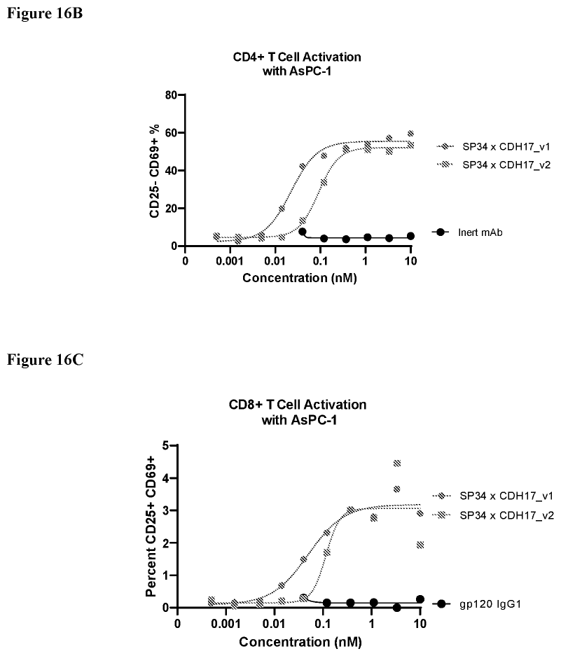

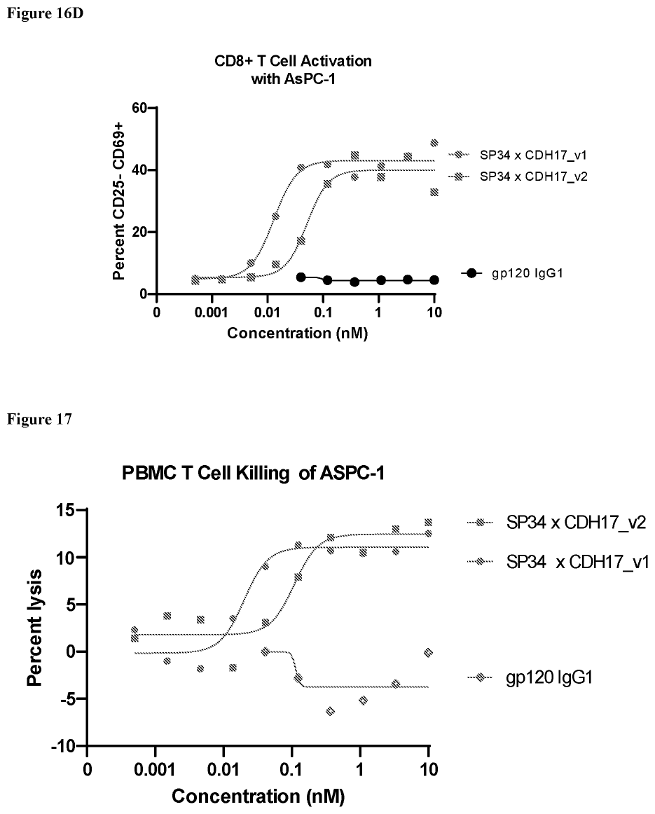

Bispecific antibodies comprising an anti-CDH17 arm paired with an anti-CD3 arm in a series of increasing concentrations were mixed with two cell lines, one a T cells isolated from a human PBMC donor and the other the AsPC-1 pancreatic cancer cell line. The CD3 arm was based on the SP34 antibody comprising variable heavy chain SEQ ID NO: 45 and variable light chain SEQ ID NO: 46. The CDH17_v1 was based on an antibody comprising variable heavy chain SEQ ID NO: 38 and variable light chain SEQ ID NO:42. The CDH17_v2 was based on an antibody comprising variable heavy chain SEQ ID NO: 39 and variable light chain SEQ ID NO:43. FIG. 16A shows CD4+ T cell activation with AsPC-1 cells with the y axis being the percent of CD25+ CD69+ T cells. FIG. 16B shows CD4+ T cell activation with AsPC-1 cells with the y axis being the percent of CD25− CD69+ T cells. FIG. 16C shows CD8+ T cell activation with AsPC-1 cells with the y axis being the percent of CD25+ CD69+ T cells. FIG. 16D shows CD4+ T cell activation with AsPC-1 cells with the y axis being the percent of CD25− CD69+ T cells. The x axes for FIG. 16A-D were the concentration of the antibodies tested. Both CD4 and CD8 positive T cells were activated, both being linked to cancer cell killing.

### Example 7: Activation of CD3+ T Cells in the Presence of Pancreatic Cancer Cell Line AsPC-1

In cancer therapy, a CDH17×CD3 bispecific antibody is expected to bind to CD3 on an attacking killer T cell and to CDH17 on a target cancer cell. FIG. 17 demonstrated dose responses of CDH17×CD3 bispecific antibody directed primary human T cell killing of AsPC-1, a CDH17 bearing pancreatic cancer cell line. The CD3 arm was based on the SP34 antibody comprising variable heavy chain SEQ ID NO: 45 and variable light chain SEQ ID NO: 46. The CDH17_v1 was based on an antibody comprising variable heavy chain SEQ ID NO: 38 and variable light chain SEQ ID NO:42. The CDH17_v2 was based on an antibody comprising variable heavy chain SEQ ID NO: 39 and variable light chain SEQ ID NO: 43. Potent T cell killing responses were observed.

### REFERENCES

Altree-Tacha, D., J. Tyrrell and T. Haas (2017). “CDH17 Is a More Sensitive Marker for Gastric Adenocarcinoma Than CK20 and CDX2.” Arch Pathol Lab Med 141 (1): 144-150. Amgen. (2020).
Amgen. (2021). “Bincyto.”
Banys-Paluchowski, M., I. Witzel, B. Aktas, P. A. Fasching, A. Hartkopf, W. Janni, S. Kasimir-Bauer, K. Pantel, G. Schon, B. Rack, S. Riethdorf, E. F. Solomayer, T. Fehm and V. Muller (2019). “The prognostic relevance of urokinase-type plasminogen activator (uPA) in the blood of patients with metastatic breast cancer.” Sci Rep 9 (1): 2318.
Bartolome, R. A., C. Aizpurua, M. Jaen, S. Torres, E. Calvino, J. I. Imbaud and J. I. Casal (2018). “Monoclonal Antibodies Directed against Cadherin RGD Exhibit Therapeutic Activity against Melanoma and Colorectal Cancer Metastasis.” Clin Cancer Res 24 (2): 433-444.
Biopharmadive.com. (2021).
Bolt, S., E. Routledge, I. Lloyd, L. Chatenoud, H. Pope, S. D. Gorman, M. Clark and H. Waldmann (1993). “The generation of a humanized, non-mitogenic CD3 monoclonal antibody which retains in vitro immunosuppressive properties.” Eur J Immunol 23 (2): 403-411.
Chen, X., J. L. Zaro and W. C. Shen (2013). “Fusion protein linkers: property, design and functionality.” Adv Drug Deliv Rev 65 (10): 1357-1369.
Chothia, C. and A. M. Lesk (1987). “Canonical structures for the hypervariable regions of immunoglobulins.” J Mol Biol 196 (4): 901-917.
Chu, S. Y., I. Vostiar, S. Karki, G. L. Moore, G. A. Lazar, E. Pong, P. F. Joyce, D. E. Szymkowski and J. R. Desjarlais (2008). “Inhibition of B cell receptor-mediated activation of primary human B cells by coengagement of CD19 and FcgammaRIIb with Fc-engineered antibodies.” Mol Immunol 45 (15): 3926-3933.
Dall'Acqua, W. F., P. A. Kiener and H. Wu (2006). “Properties of human IgG1s engineered for enhanced binding to the neonatal Fc receptor (FcRn).” J Biol Chem 281 (33): 23514-23524.
Diebolder, C. A., F. J. Beurskens, R. N. de Jong, R. I. Koning, K. Strumane, M. A. Lindorfer, M. Voorhorst, D. Ugurlar, S. Rosati, A. J. Heck, J. G. van de Winkel, I. A. Wilson, A. J. Koster, R. P. Taylor, E. O. Saphire, D. R. Burton, J. Schuurman, P. Gros and P. W. Parren (2014). “Complement is activated by IgG hexamers assembled at the cell surface.” Science 343 (6176): 1260-1263.
Dong, W., Q. Yu and Y. Xu (2007). “Altered expression of a Li-cadherin in gastric cancer and intestinal metaplasia.” Dig Dis Sci 52 (2): 536-542.
EMA. (2021).
Gunasekaran, K., M. Pentony, M. Shen, L. Garrett, C. Forte, A. Woodward, S. B. Ng, T. Born, M. Retter, K. Manchulenko, H. Sweet, I. N. Foltz, M. Wittekind and W. Yan (2010). “Enhancing antibody Fc heterodimer formation through electrostatic steering effects: applications to bispecific molecules and monovalent IgG.” J Biol Chem 285 (25): 19637-19646.
Heiss, M. M., P. Murawa, P. Koralewski, E. Kutarska, O. O. Kolesnik, V. V. Ivanchenko, A. S. Dudnichenko, B. Aleknaviciene, A. Razbadauskas, M. Gore, E. Ganea-Motan, T. Ciuleanu, P. Wimberger, A. Schmittel, B. Schmalfeldt, A. Burges, C. Bokemeyer, H. Lindhofer, A. Lahr and S. L. Parsons (2010). “The trifunctional antibody catumaxomab for the treatment of malignant ascites due to epithelial cancer: Results of a prospective randomized phase II/III trial.” Int J Cancer 127 (9): 2209-2221.
Hinton, P. R., J. M. Xiong, M. G. Johlfs, M. T. Tang, S. Keller and N. Tsurushita (2006). “An engineered human IgG1 antibody with longer serum half-life.” J Immunol 176 (1): 346-356.
Kinder, M., A. R. Greenplate, K. D. Grugan, K. L. Soring, K. A. Heeringa, S. G. McCarthy, G. Bannish, M. Perpetua, F. Lynch, R. E. Jordan, W. R. Strohl and R. J. Brezski (2013). “Engineered protease-resistant antibodies with selectable cell-killing functions.” J Biol Chem 288 (43): 30843-30854.
Ko, S., K. M. Chu, J. M. Luk, B. W. Wong, S. T. Yuen, S. Y. Leung and J. Wong (2004). “Overexpression of LI-cadherin in gastric cancer is associated with lymph node metastasis.” Biochem Biophys Res Commun 319 (2): 562-568.
Kusano-Arai, O., H. Iwanari, S. Kudo, C. Kikuchi, A. Yui, H. Akiba, K. Matsusaka, A. Kaneda, M. Fukayama, K. Tsumoto and T. Hamakubo (2018). “Synergistic Cytotoxic Effect on Gastric Cancer Cells of an Immunotoxin Cocktail in Which Antibodies Recognize Different Epitopes on CDH17.” Monoclon Antib Immunodiagn Immunother 37 (1): 1-11.
Labrijn, A. F., J. I. Meesters, B. E. de Goeij, E. T. van den Bremer, J. Neijssen, M. D. van Kampen, K. Strumane, S. Verploegen, A. Kundu, M. J. Gramer, P. H. van Berkel, J. G. van de Winkel, J. Schuurman and P. W. Parren (2014). “Efficient generation of stable bispecific IgG1 by controlled Fab-arm exchange.” Proc Natl Acad Sci USA 110(13): 5145-5150.
Lee, H. J., K. T. Nam, H. S. Park, M. A. Kim, B. J. Lafleur, H. Aburatani, H. K. Yang, W. H. Kim and J. R. Goldenring (2010). “Gene expression profiling of metaplastic lineages identifies CDH17 as a prognostic marker in early stage gastric cancer.” Gastroenterology 139 (1): 213-225 e213.
Lefranc, M. P., C. Pommie, M. Ruiz, V. Giudicelli, E. Foulquier, L. Truong, V. Thouvenin-Contet and G. Lefranc (2003). “IMGT unique numbering for immunoglobulin and T cell receptor variable domains and Ig superfamily V-like domains.” Dev Comp Immunol 27 (1): 55-77.
Li, R., H. Q. Yang, H. L. Xi, S. Feng and R. H. Qin (2017). “Inhibition of CDH17 gene expression via RNA interference reduces proliferation and apoptosis of human MKN28 gastric cancer cells.” Int J Oncol 50 (1): 15-22.
Lin, W. W., Y. C. Lu, C. H. Chuang and T. L. Cheng (2020). “Ab locks for improving the selectivity and safety of antibody drugs.” J Biomed Sci 27 (1): 76.
Lin, Z., C. Zhang, M. Zhang, D. Xu, Y. Fang, Z. Zhou, X. Chen, N. Qin and X. Zhang (2014). “Targeting cadherin-17 inactivates Ras/Raf/MEK/ERK signaling and inhibits cell proliferation in gastric cancer.” PLOS One 9 (1): e85296.
Liu, X., Y. Huang, H. Yuan, X. Qi, Y. Manjunath, D. Avella, J. T. Kaifi, Y. Miao, M. Li, K. Jiang and G. Li (2019). “Disruption of oncogenic liver-intestine cadherin (CDH17) drives apoptotic pancreatic cancer death.” Cancer Lett 454:204-214.
Lutterbuese, R., T. Raum, R. Kischel, P. Hoffmann, S. Mangold, B. Rattel, M. Friedrich, O. Thomas, G. Lorenczewski, D. Rau, E. Schaller, I. Herrmann, A. Wolf, T. Urbig, P. A. Baeuerle and P. Kufer (2010). “T cell-engaging BiTE antibodies specific for EGFR potently eliminate KRAS- and BRAF-mutated colorectal cancer cells.” Proc Natl Acad Sci USA 107 (28): 12605-12610.
Marshall, J. F. (2018). “Targeting CDH17 in Cancer: When Blocking the Ligand Beats Blocking the Receptor?” Clin Cancer Res 24 (2): 253-255.
Petkova, S. B., S. Akilesh, T. J. Sproule, G. J. Christianson, H. Al Khabbaz, A. C. Brown, L. G. Presta, Y. G. Meng and D. C. Roopenian (2006). “Enhanced half-life of genetically engineered human IgG1 antibodies in a humanized FcRn mouse model: potential application in humorally mediated autoimmune disease.” Int Immunol 18 (12): 1759-1769.
Phillips, T. J., A. J. Olszewski, J. Munoz, T. M. Kim, D. H. Yoon, R. Greil, J. Westin, U. Jaeger, M. Canales, C. Chen, B. Althaus, C. O'Hear, R. Negricea, Y. Xie, R. McCord, E. Purev and A. Vallurupalli (2020). “Mosunetuzumab, a Novel CD20/CD3 Bispecific Antibody, in Combination with CHOP Confers High Response Rates in Patients with Diffuse Large B-Cell Lymphoma.” Blood (2020) 136 (Supplement 1): 37-38, 136: 37-38.
Qiu, H. B., L. Y. Zhang, C. Ren, Z. L. Zeng, W. J. Wu, H. Y. Luo, Z. W. Zhou and R. H. Xu (2013). “Targeting CDH17 suppresses tumor progression in gastric cancer by downregulating Wnt/beta-catenin signaling.” PLOS One 8 (3): e56959.
Ridgway, J. B., L. G. Presta and P. Carter (1996). “Knobs-into-holes' engineering of antibody CH3 domains for heavy chain heterodimerization.” Protein Eng 9 (7): 617-621.
Scheraga, H. A. (2008). “From helix-coil transitions to protein folding.” Biopolymers 89 (5): 479-485.
Shields, R. L., A. K. Namenuk, K. Hong, Y. G. Meng, J. Rae, J. Briggs, D. Xie, J. Lai, A. Stadlen, B. Li, J. A. Fox and L. G. Presta (2001). “High resolution mapping of the binding site on human IgG1 for Fc gamma RI, Fc gamma RII, Fc gamma RIII, and FcRn and design of IgG1 variants with improved binding to the Fc gamma R.” J Biol Chem 276 (9): 6591-6604.
Shields, R. L., A. K. Namenuk, K. Hong, Y. G. Meng, J. Rae, J. Briggs, D. Xie, J. Lai, A. Stadlen, B. Li, J. A. Fox and L. G. Presta (2001). “High resolution mapping of the binding site on human IgG1 for Fc gamma RI, Fc gamma RII, Fc gamma RIII, and FcRn and design of IgG1 variants with improved binding to the Fc gamma R.” J Biol Chem 276 (9): 6591-6604.
Singh, A., S. Dees and I. S. Grewal (2021). “Overcoming the challenges associated with CD3+ T-cell redirection in cancer.” Br J Cancer 124 (6): 1037-1048.
Snow, A. N., S. Mangray, S. Lu, R. Clubwala, J. Li, M. B. Resnick and E. Yakirevich (2015). “Expression of cadherin 17 in well-differentiated neuroendocrine tumours.” Histopathology 66 (7): 1010-1021.
Su, A. I., T. Wiltshire, S. Batalov, H. Lapp, K. A. Ching, D. Block, J. Zhang, R. Soden, M. Hayakawa, G. Kreiman, M. P. Cooke, J. R. Walker and J. B. Hogenesch (2004). “A gene atlas of the mouse and human protein-encoding transcriptomes.” Proc Natl Acad Sci USA 101 (16): 6062-6067.
Su, M. C., R. H. Yuan, C. Y. Lin and Y. M. Jeng (2008). “Cadherin-17 is a useful diagnostic marker for adenocarcinomas of the digestive system.” Mod Pathol 21 (11): 1379-1386.
Sun, L. L., D. Ellerman, M. Mathieu, M. Hristopoulos, X. Chen, Y. Li, X. Yan, R. Clark, A. Reyes, E. Stefanich, E. Mai, J. Young, C. Johnson, M. Huseni, X. Wang, Y. Chen, P. Wang, H. Wang, N. Dybdal, Y. W. Chu, N. Chiorazzi, J. M. Scheer, T. Junttila, K. Totpal, M. S. Dennis and A. J. Ebens (2015). “Anti-CD20/CD3 T cell-dependent bispecific antibody for the treatment of B cell malignancies.” Sci Transl Med 7 (287): 287ra270.
Takamura, M., M. Sakamoto, Y. Ino, T. Shimamura, T. Ichida, H. Asakura and S. Hirohashi (2003). “Expression of liver-intestine cadherin and its possible interaction with galectin-3 in ductal adenocarcinoma of the pancreas.” Cancer Sci 94 (5): 425-430.
Tam, S. H., S. G. McCarthy, A. A. Armstrong, S. Somani, S. J. Wu, X. Liu, A. Gervais, R. Ernst, D. Saro, R. Decker, J. Luo, G. L. Gilliland, M. L. Chiu and B. J. Scallon (2017). “Functional, Biophysical, and Structural Characterization of Human IgG1 and IgG4 Fc Variants with Ablated Immune Functionality.” Antibodies (Basel) 6 (3).
Tian, X., Z. Han, Q. Zhu, J. Tan, W. Liu, Y. Wang, W. Chen, Y. Zou, Y. Cai, S. Huang, A. Chen, T. Zhan, M. Huang, M. Liu and X. Huang (2018). “Silencing of cadherin-17 enhances apoptosis and inhibits autophagy in colorectal cancer cells.” Biomed Pharmacother 108:331-337.
Vafa, O., G. L. Gilliland, R. J. Brezski, B. Strake, T. Wilkinson, E. R. Lacy, B. Scallon, A. Teplyakov, T. J. Malia and W. R. Strohl (2014). “An engineered Fc variant of an IgG eliminates all immune effector functions via structural perturbations.” Methods 65 (1): 114-126.
van Roy, F. (2014). “Beyond E-cadherin: roles of other cadherin superfamily members in cancer.” Nat Rev Cancer 14 (2): 121-134.
Viardot, A., M. E. Goebeler, G. Hess, S. Neumann, M. Pfreundschuh, N. Adrian, F. Zettl, M. Libicher, C. Sayehli, J. Stieglmaier, A. Zhang, D. Nagorsen and R. C. Bargou (2016). “Phase 2 study of the bispecific T-cell engager (BiTE) antibody blinatumomab in relapsed/refractory diffuse large B-cell lymphoma.” Blood 127 (11): 1410-1416.
Wikipedia. (2021). “Catumaxomab.”
Wong, B. W., J. M. Luk, I. O. Ng, M. Y. Hu, K. D. Liu and S. T. Fan (2003). “Identification of liver-intestine cadherin in hepatocellular carcinoma—a potential disease marker.” Biochem Biophys Res Commun 311 (3): 618-624.
Worn, A. and A. Pluckthun (2001). “Stability engineering of antibody single-chain Fv fragments.” J Mol Biol 305 (5): 989-1010.
Wu, C., X. Jin, G. Tsueng, C. Afrasiabi and A. I. Su (2016). “BioGPS: building your own mash-up of gene annotations and expression profiles.” Nucleic Acids Res 44 (D1): D313-316.
Wu, T. T. and E. A. Kabat (1970). “An analysis of the sequences of the variable regions of Bence Jones proteins and myeloma light chains and their implications for antibody complementarity.” J Exp Med 132 (2): 211-250.
Xu, D., M. L. Alegre, S. S. Varga, A. L. Rothermel, A. M. Collins, V. L. Pulito, L. S. Hanna, K. P. Dolan, P. W. Parren, J. A. Bluestone, L. K. Jolliffe and R. A. Zivin (2000). “In vitro characterization of five humanized OKT3 effector function variant antibodies.” Cell Immunol 200 (1): 16-26.
Zalevsky, J., A. K. Chamberlain, H. M. Horton, S. Karki, I. W. Leung, T. J. Sproule, G. A. Lazar, D. C. Roopenian and J. R. Desjarlais (2010). “Enhanced antibody half-life improves in vivo activity.” Nat Biotechnol 28 (2): 157-159.
Zhang, D., A. A. Armstrong, S. H. Tam, S. G. McCarthy, J. Luo, G. L. Gilliland and M. L. Chiu (2017). “Functional optimization of agonistic antibodies to OX40 receptor with novel Fc mutations to promote antibody multimerization.” MAbs 9:1129-1142.

1. An anti-CDH17 antibody or an antigen-binding fragment thereof, comprising:
a heavy chain variable region comprising HCDR1, HCDR2, and HCDR3, wherein the HCDR1, HCDR2, and HCDR3 are selected from: SEQ ID NO: 65, SEQ ID NO: 66, and SEQ ID NO: 67; and SEQ ID NO: 68, SEQ ID NO: 69, and SEQ ID NO: 70; respectively; and a light chain variable region comprising LCDR1, LCDR2, and LCDR3, wherein the LCDR1, LCDR2, and LCDR3 are selected from: SEQ ID NO: 62, SEQ ID NO: 63, and SEQ ID NO: 64; and SEQ ID NO: 71, SEQ ID NO: 72, and SEQ ID NO: 73; respectively.

2. The anti-CDH17 antibody or antigen binding fragment of claim 1, comprising a heavy chain sequence comprising an amino acid sequence having at least 85% identity to any one of SEQ ID NOs: 3 and 5 or an antigen-binding fragment thereof, and a light chain sequence comprising an amino acid sequence having at least 85% identity to any one of SEQ ID NOs: 2 and 6 or an antigen-binding fragment thereof.

3. An anti-CDH17 antibody or antigen binding fragment thereof, comprising a light chain sequence and a heavy chain sequence comprising: SEQ ID NOs: 3 and 2; SEQ ID NOs: 4 and 2; SEQ ID NOs: 5 and 6; SEQ ID NOs: 7 and 9; or SEQ ID NOs: 8 and 9;
respectively.

4. An anti-CDH17 antibody or an antigen-binding fragment thereof, comprising: at least one variable-heavy-chain-only single-domain or an antigen-binding fragment thereof, wherein the at least one variable-heavy-chain-only single-domain comprises HCDR1, HCDR2, and HCDR3 selected from:
SEQ ID NO: 74, SEQ ID NO: 75, and SEQ ID NO: 76;
SEQ ID NO: 77, SEQ ID NO: 78, and SEQ ID NO: 79;
SEQ ID NO: 80, SEQ ID NO: 81, and SEQ ID NO: 82;
SEQ ID NO: 77, SEQ ID NO: 83, and SEQ ID NO: 84;
SEQ ID NO: 77, SEQ ID NO: 81, and SEQ ID NO: 85;
SEQ ID NO: 86, SEQ ID NO: 87, and SEQ ID NO: 88;
SEQ ID NO: 86, SEQ ID NO: 89, and SEQ ID NO: 90;
SEQ ID NO: 77, SEQ ID NO: 91, and SEQ ID NO: 92;
SEQ ID NO: 93, SEQ ID NO: 75, and SEQ ID NO: 94;
SEQ ID NO: 77, SEQ ID NO: 95, and SEQ ID NO: 96;
SEQ ID NO: 77, SEQ ID NO: 97, and SEQ ID NO: 98;
SEQ ID NO: 77, SEQ ID NO: 99, and SEQ ID NO: 100;
SEQ ID NO: 86, SEQ ID NO: 101, and SEQ ID NO: 102;
SEQ ID NO: 103, SEQ ID NO: 104, and SEQ ID NO: 105;
SEQ ID NO: 86, SEQ ID NO: 106, and SEQ ID NO: 107;
SEQ ID NO: 108, SEQ ID NO: 109, and SEQ ID NO: 110;
SEQ ID NO: 77, SEQ ID NO: 111, and SEQ ID NO: 112;
SEQ ID NO: 77, SEQ ID NO: 113, and SEQ ID NO: 114;
SEQ ID NO: 115, SEQ ID NO: 116, and SEQ ID NO: 117;
SEQ ID NO: 77, SEQ ID NO: 118, and SEQ ID NO: 119;
SEQ ID NO: 120, SEQ ID NO: 78, and SEQ ID NO: 121;
SEQ ID NO: 77, SEQ ID NO: 101, and SEQ ID NO: 122;
SEQ ID NO: 115, SEQ ID NO: 123, and SEQ ID NO: 124;
SEQ ID NO: 115, SEQ ID NO: 125, and SEQ ID NO: 126;
SEQ ID NO: 115, SEQ ID NO: 127, and SEQ ID NO: 128;
SEQ ID NO: 77, SEQ ID NO: 101, and SEQ ID NO: 129;
SEQ ID NO: 130, SEQ ID NO: 131, and SEQ ID NO: 132; and
SEQ ID NO: 115, SEQ ID NO: 133, and SEQ ID NO: 134; respectively.

5. The anti-CDH17 antibody or antigen binding fragment of claim 4, comprising at least one variable-heavy-chain-only single-domain or an antigen-binding fragment thereof, wherein the at least one variable-heavy-chain-only single-domain comprises an amino acid sequence having at least 85% identity to any one of SEQ ID NOs: 10-37 or an antigen-binding fragment thereof.

6. An anti-CD3 and anti-CDH17 bispecific antibody, comprising:
(1) a first binding arm comprising:
a first heavy chain fusion protein comprising, from the N- to the C-terminus, an optional shield A, an optional protease sequence A, and an IgG heavy chain or an antigen-binding fragment thereof, and
a first light chain fusion protein comprising, from the N- to the C-terminus, an optional shield B, an optional protease sequence B, and an IgG light chain or an antigen-binding fragment thereof,
wherein:
the IgG heavy chain or antigen-binding fragment thereof of the first binding arm comprises HCDR1, HCDR2, and HCDR3 selected from: SEQ ID NO: 135, SEQ ID NO: 136, and SEQ ID NO: 137; SEQ ID NO: 135, SEQ ID NO: 138, and SEQ ID NO: 137; and SEQ ID NO: 135, SEQ ID NO: 139, and SEQ ID NO: 137; respectively, and
the IgG light chain or antigen-binding fragment thereof of the first binding arm comprises LCDR1, LCDR2, and LCDR3 selected from: SEQ ID NO: 140, SEQ ID NO: 141, and SEQ ID NO: 142; and SEQ ID NO: 143, SEQ ID NO: 141, and SEQ ID NO: 142; respectively; and
(2) a second binding arm comprising:
a second heavy chain fusion protein comprising, from the N- to the C-terminus, an optional shield C, an optional protease sequence C, and an IgG heavy chain or an antigen-binding fragment thereof; and
a second light chain fusion protein comprising, from the N- to the C-terminus, an optional shield D, an optional protease sequence D, and an IgG light chain or an antigen-binding fragment thereof,
wherein:
the IgG heavy chain or antigen-binding fragment thereof of the second binding arm comprises HCDR1, HCDR2, and HCDR3 selected from: SEQ ID NO: 65, SEQ ID NO: 66, and SEQ ID NO: 67; and SEQ ID NO: 68, SEQ ID NO: 69, and SEQ ID NO: 70; respectively; and
the IgG light chain or antigen-binding fragment thereof of the second binding arm comprises LCDR1, LCDR2, and LCDR3 selected from: SEQ ID NO: 62, SEQ ID NO: 63, and SEQ ID NO: 64; and SEQ ID NO: 71, SEQ ID NO: 72, and SEQ ID NO: 73; respectively; and
wherein the shields A-D are the same or different from one another, and protease sequences A-D are the same or different from one another.

7. The anti-CD3 and anti-CDH17 bispecific antibody of claim 6, wherein:
the IgG heavy chain or antigen-binding fragment thereof of the first binding arm comprises an amino acid sequence having at least 85% identity to any one of SEQ ID NOs: 39-41 or an antigen-binding fragment thereof,
the IgG light chain or antigen-binding fragment thereof of the first binding arm comprises a light chain sequence comprising an amino acid sequence having at least 85% identity to any one of SEQ ID NOs: 43-44 or an antigen-binding fragment thereof; and
wherein:
the IgG heavy chain or antigen-binding fragment thereof of the second binding arm comprises a heavy chain sequence comprising an amino acid sequence having at least 85% identity to any one of SEQ ID NOs: 3 and 5 or an antigen-binding fragment thereof, and
the IgG light chain or antigen-binding fragment thereof of the second binding arm comprises a light chain sequence comprising an amino acid sequence with at least 85% identity to any one of SEQ ID NOs: 2 and 6 or an antigen-binding fragment thereof.

8. An anti-CD3 and anti-CDH17 bispecific antibody comprising:
(1) a first binding arm comprising:
a first heavy chain fusion protein comprising, from the N- to the C-terminus, an optional shield A, an optional protease sequence A, and an IgG heavy chain or an antigen-binding fragment thereof, and
a first light chain fusion protein comprising, from the N- to the C-terminus, an optional shield B, an optional protease sequence B, and an IgG light chain or an antigen-binding fragment thereof,
wherein:
the IgG heavy chain or antigen-binding fragment thereof of the first binding arm comprises HCDR1, HCDR2, and HCDR3 selected from: SEQ ID NO: 135, SEQ ID NO: 136, and SEQ ID NO: 137; SEQ ID NO: 135, SEQ ID NO: 138, and SEQ ID NO: 137; and SEQ ID NO: 135, SEQ ID NO: 139, and SEQ ID NO: 137; respectively, and
the IgG light chain or antigen-binding fragment thereof of the first binding arm comprises LCDR1, LCDR2, and LCDR3 selected from: SEQ ID NO: 140, SEQ ID NO: 141, and SEQ ID NO: 142; and SEQ ID NO: 143, SEQ ID NO: 141, and SEQ ID NO: 142; respectively; and
(2) a second binding arm comprising:
a second heavy chain fusion protein comprising, from the N- to the C-terminus, an optional shield C, an optional protease sequence C, and an IgG heavy chain comprising at least one variable-heavy-chain-only single domain or an antigen-binding fragment thereof,
wherein the at least one variable-heavy-chain-only single domain or antigen-binding fragment thereof comprises HCDR1, HCDR2, and HCDR3 selected from:
SEQ ID NO: 74, SEQ ID NO: 75, and SEQ ID NO: 76;
SEQ ID NO: 77, SEQ ID NO: 78, and SEQ ID NO: 79;
SEQ ID NO: 80, SEQ ID NO: 81, and SEQ ID NO: 82;
SEQ ID NO: 77, SEQ ID NO: 83, and SEQ ID NO: 84;
SEQ ID NO: 77, SEQ ID NO: 81, and SEQ ID NO: 85;
SEQ ID NO: 86, SEQ ID NO: 87, and SEQ ID NO: 88;
SEQ ID NO: 86, SEQ ID NO: 89, and SEQ ID NO: 90;
SEQ ID NO: 77, SEQ ID NO: 91, and SEQ ID NO: 92;
SEQ ID NO: 93, SEQ ID NO: 75, and SEQ ID NO: 94;
SEQ ID NO: 77, SEQ ID NO: 95, and SEQ ID NO: 96;
SEQ ID NO: 77, SEQ ID NO: 97, and SEQ ID NO: 98;
SEQ ID NO: 77, SEQ ID NO: 99, and SEQ ID NO: 100;
SEQ ID NO: 86, SEQ ID NO: 101, and SEQ ID NO: 102;
SEQ ID NO: 103, SEQ ID NO: 104, and SEQ ID NO: 105;
SEQ ID NO: 86, SEQ ID NO: 106, and SEQ ID NO: 107;
SEQ ID NO: 108, SEQ ID NO: 109, and SEQ ID NO: 110;
SEQ ID NO: 77, SEQ ID NO: 111, and SEQ ID NO: 112;
SEQ ID NO: 77, SEQ ID NO: 113, and SEQ ID NO: 114;
SEQ ID NO: 115, SEQ ID NO: 116, and SEQ ID NO: 117;
SEQ ID NO: 77, SEQ ID NO: 118, and SEQ ID NO: 119;
SEQ ID NO: 120, SEQ ID NO: 78, and SEQ ID NO: 121;
SEQ ID NO: 77, SEQ ID NO: 101, and SEQ ID NO: 122;
SEQ ID NO: 115, SEQ ID NO: 123, and SEQ ID NO: 124;
SEQ ID NO: 115, SEQ ID NO: 125, and SEQ ID NO: 126;
SEQ ID NO: 115, SEQ ID NO: 127, and SEQ ID NO: 128;
SEQ ID NO: 77, SEQ ID NO: 101, and SEQ ID NO: 129;
SEQ ID NO: 130, SEQ ID NO: 131, and SEQ ID NO: 132; and
SEQ ID NO: 115, SEQ ID NO: 133, and SEQ ID NO: 134; respectively, and
wherein the shields A-C are the same or different from one another, and protease sequences A-C are the same or different from one another.

9. The anti-CD3 and anti-CDH17 bispecific antibody of claim 8, wherein:
the IgG heavy chain or antigen-binding fragment thereof of the first binding arm comprises an amino acid sequence having at least 85% identity to any one of SEQ ID NOs: 39-41 or an antigen-binding fragment thereof,
the IgG light chain or antigen-binding fragment thereof of the first binding arm comprises a light chain sequence comprising an amino acid sequence having at least 85% identity to any one of SEQ ID NOs: 43-44 or an antigen-binding fragment thereof; and
wherein:
the at least one variable-heavy-chain-only single domain or antigen-binding fragment thereof comprises an amino acid sequence having at least 85% identity to any one of SEQ ID NOs: 10-37 or an antigen-binding fragment thereof.

10. The bispecific antibody of claim 6, wherein the first binding arm is monovalent, and the second binding arm is monovalent, bivalent, or multivalent.

11. The bispecific antibody of claim 6, wherein the shield A, shield B, shield C, and shield D are each independently selected from the amino acid sequences set forth in SEQ ID NOs: 47-51.

12. The bispecific antibody of claim 6, wherein the protease cleavable linker A, protease cleavable linker B, protease cleavable linker C, and protease cleavable linker D are each independently selected from amino acid sequences set forth in SEQ ID NOs: 52-59.

13. A bispecific antibody comprising a CD3-targeting binding arm, wherein the CD3-targeting binding arm comprises a heavy chain sequence and a light chain sequence comprising SEQ ID NOs: 38 and 42; SEQ ID NOs: 38 and 43; SEQ ID NOs: 38 and 44; SEQ ID NOs: 39 and 42; SEQ ID NOs: 39 and 43; SEQ ID NOs: 39 and 44; SEQ ID NOs: 40 and 42; SEQ ID NOs: 40 and 43; SEQ ID NOs: 40 and 44; SEQ ID NOs: 41 and 42; SEQ ID NOs: 41 and 43; or SEQ ID NOs: 41 and 44; respectively.

14. The bispecific antibody of claim 13, further comprising a CDH17-targeting binding arm, wherein the CDH17-targeting binding arm comprises a heavy chain sequence and a light chain sequence comprising SEQ ID NOs: 3 and 2; SEQ ID NOs: 4 and 2; SEQ ID NOs: 5 and 6; SEQ ID NOs: 7 and 9; or SEQ ID NOs: 8 and 9; respectively.

15. The bispecific antibody of claim 6, wherein the bispecific antibody comprises a modified F_{c} to extend the half-life of the bispecific antibody, enhance resistance of the bispecific antibody to proteolytic degradation, reduce effector functionality of the bispecific antibody, facilitate generation of the bispecific antibody by F_{c} heterodimerization, facilitate multimerization of the bispecific antibody, and/or improve manufacturing and drug stability of the bispecific antibody.

16. A conjugate comprising the anti-CDH17 antibody of claim 1 conjugated to a cytotoxic agent.

17. A composition comprising the anti-CDH17 antibody of claim 1.

18. A pharmaceutical composition comprising the anti-CDH17 antibody of claim 1, and a pharmaceutically acceptable carrier.

19. A nucleic acid encoding the anti-CDH17 antibody of claim 1.

20. A recombinant expression vector comprising the nucleic acid of claim 19.

21. A host cell comprising a nucleic acid of claim 19.

22. A method for preparing an anti-CDH17 antibody, comprising: culturing the host cell of claim 21, growing the host cell in a host cell culture, providing host cell culture conditions wherein the nucleic acid is expressed, and recovering the anti-CDH17 antibody or the bispecific antibody from the host cell or from the host cell culture.

23. The method for preparing an anti-CDH17 antibody of claim 22, wherein the bispecific antibody is obtained using controlled Fab arm exchange.

24. A method for treating or preventing a CDH17-mediated disease or disorder in a subject in need thereof, comprising administering to the subject a pharmaceutically effective amount of the anti-CDH17 antibody of claim 1.

25. The method for treating or preventing a CDH17-mediated disease or disorder of claim 24, wherein the CDH17-mediated disease or disorder is lung cancer, breast cancer, colorectal cancer, or gastric cancer.

26. The method for treating or preventing a CDH17-mediated disease or disorder of claim 24, wherein the subject suffers from relapsed CDH17 positive cancer.


SEQUENCE LISTING (147 sequences)

SEQ ID NO: 1 - protein, 832 aa, Homo sapiens
  REGION 1..832: note = primary amino acid sequence of Cadherin-17
MILQAHLHSLCLLMLYLATGYGQEGKFSGPLKPMTFSIYEGQEPSQIIFQFKANPPAVTFELTGETDNIFVIEREGLLYYNRALDRETRSTHNLQVAALDANGIIVEGPVPITIKVKDINDNRPTFLQSKYEGSVRQNSRPGKPFLYVNATDLDDPATPNGQLYYQIVIQLPMINNVMYFQINNKTGAISLTREGSQELNPAKNPSYNLVISVKDMGGQSENSFSDTTSVDIIVTENIWKAPKPVEMVENSTDPHPIKITQVRWNDPGAQYSLVDKEKLPRFPFSIDQEGDIYVTQPLDREEKDAYVFYAVAKDEYGKPLSYPLEIHVKVKDINDNPPTCPSPVTVFEVQENERLGNSIGTLTAHDRDEENTANSFLNYRIVEQTPKLPMDGLFLIQTYAGMLQLAKQSLKKQDTPQYNLTIEVSDKDFKTLCFVQINVIDINDQIPIFEKSDYGNLTLAEDTNIGSTILTIQATDADEPFTGSSKILYHIIKGDSEGRLGVDTDPHTNTGYVIIKKPLDFETAAVSNIVFKAENPEPLVFGVKYNASSFAKFTLIVTDVNEAPQFSQHVFQAKVSEDVAIGTKVGNVTAKDPEGLDISYSLRGDTRGWLKIDHVTGEIFSVAPLDREAGSPYRVQVVATEVGGSSLSSVSEFHLILMDVNDNPPRLAKDYTGLFFCHPLSAPGSLIFEATDDDQHLFRGPHFTFSLGSGSLQNDWEVSKINGTHARLSTRHTEFEEREYVVLIRINDGGRPPLEGIVSLPVTFCSCVEGSCFRPAGHQTGIPTVGMAVGILLTTLLVIGIILAVVFIRIKKDKGKDNVESAQASEVKPLRS

SEQ ID NO: 2 - protein, 113 aa, synthetic construct
  REGION 1..113: note = CDH17 LC1
DIVMSQSPSSLAVSVGEKVTMSCKSSQSLLHSSNQKNYLAWYQQKPGQSPKVLIYWASTRESGVPDRFTGSGSGTDFTLTITSVKSEDLAVYYCQQYYSYPWTFGGGTRLEIK

SEQ ID NO: 3 - protein, 123 aa, synthetic construct
  REGION 1..123: note = CDH17 HC1
EVQLQQSVAELVKPGASVKMSCKVSGYTLTDHTIHWMKQRPEQGLEWIGYIYPRDGITGYNEKFKGKATLTADTSSSTAYMQLNSLTSEDSAVYFCARWGYSYRNYAYYYDYWGQGTTLTVSS

SEQ ID NO: 4 - protein, 123 aa, synthetic construct
  REGION 1..123: note = CDH17 H2 HC0
QVQLVQSGAEVKKPGSSVKVSCKVSGYTFTDHTIHWMKQRPGQGLEWIGYIYPRDVITQYNEKFKGKVTLTADKSTSTAYMELSSLRSEDTAVYLCARWGYFYGSRSYYFDYWGQGTTVTVSS

SEQ ID NO: 5 - protein, 123 aa, synthetic construct
  REGION 1..123: note = CDH17 E2 HC
QVQLQQSDAELVKPGASVKISCKVSGYTFTDHTIHWMKQRPEQGLEWIGYIYPRDVITQYNEKFKGKATLTADKSSSTAYMQLNSLTSEDSAVYLCARWGYFYGSRSYYFDYWGQGTTLTVSS

SEQ ID NO: 6 - protein, 107 aa, synthetic construct
  REGION 1..107: note = CDH17 E2 LC
DIVMTQSQKFMSTTVGDRVNITCKASQNVGTAVAWYQQKPGQSPKLLIYSASNRYTGVPDRFTGSGSGTDFTLTITNMQSKDLADYFCQQYSSYPWTFGGGTKLEIK

SEQ ID NO: 7 - protein, 122 aa, synthetic construct
  REGION 1..122: note = CDH17 M1 HC
EVQLQQSVAELVKPGASVKMSCKVSGYTLTDHTIHMKQRPEQGLEWIGYIYPRDGITGYNEKFKGKATLTADTSSSTAYMQLNSLTSEDSAVYFCARWGYSYRNYAYYYDYWGQGTTLTVSS

SEQ ID NO: 8 - protein, 117 aa, synthetic construct
  REGION 1..117: note = CDH17 M3 HC
EVQLVESGGGLVKPGGSLKLSCAASGFSFSDYYMYWVRQAPEKRLEWVASISFDGTYTYYTDRVKGRFTISRDNAKNNLYLQMSSLKSEDTAMYYCARDRPAWFPYWGQGTLVTVSA

SEQ ID NO: 9 - protein, 114 aa, synthetic construct
  REGION 1..114: note = CDH17 M3 LC
DVLMTQIPLSLTVSLGDQASISCRSSQSIVHSNGNTYLEWYLQRPGPSPKLLIYKVSNRFSGVPDRFSGSGSGSGTDFTLKISRVEAEDLGVYYCFQGSHVPLTFGAGTKLELK

SEQ ID NO: 10 - protein, 119 aa, synthetic construct
  REGION 1..119: note = TS00349
EVQLLESGGGLVQPGGSLRLSCAASGFTFSDYWMHWVRQAPGKGLEWVSAIWSSGGSTYYADSVKGRFTISRDNSKNTLYLQMNSLRAEDTAVYYCAKSPTLYDDFDYWGQGTLVTVSS

SEQ ID NO: 11 - protein, 124 aa, synthetic construct
  REGION 1..124: note = TS00369
EVQLLESGGGLVQPGGSLRLSCAASGFTFSDYWMSWVRQAPGKGLEWVSNISGSGSSTYYADSVKGRFTISRDNSKNTLYLQMNSLRAEDTAVYYCAKTSQWLARERSSEFDYWGQGTLVTVSS

SEQ ID NO: 12 - protein, 121 aa, synthetic construct
  REGION 1..121: note = TS00347
EVQLLESGGGLVQPGGSLRLSCAASGFTFSTYAMSWVRQAPGKGLEWVSNISGSGGSTYYADSVKGRFTISRDNSKNTLYLQMNSLRAEDTAVYYCAKEQQWVPGFGFDYWGQGTLVTVSS

SEQ ID NO: 13 - protein, 121 aa, synthetic construct
  REGION 1..121: note = TS00366
EVQLLESGGGLVQPGGSLRLSCAASGFTFSDYWMSWVRQAPGKGLEWVSNINGDGGSTYYADSVKGRFTISRDNSKNTLYLQMNSLRAEDTAVYYCAKTSTFYKQDKFDYWGQGTLVTVSS

SEQ ID NO: 14 - protein, 124 aa, synthetic construct
  REGION 1..124: note = TS00364
EVQLLESGGGLVQPGGSLRLSCAASGFTFSDYWMSWVRQAPGKGLEWVSNISGSGGSTYYADSVKGRFTISRDNSKNTLYLQMNSLRAEDTAVYYCAKTSDWLLHDADLHFDYWGQGTLVTVSS

SEQ ID NO: 15 - protein, 124 aa, synthetic construct
  REGION 1..124: note = TS00363
EVQLLESGGGLVQPGGSLRLSCAASGFTFSTYWMSWVRQAPGKGLEWVSNISQDGGSTYYADSVKGRFTISRDNSKNTLYLQMNSLRAEDTAVYYCAKTTDWLQDKVDEAFDYWGQGTLVTVSS

SEQ ID NO: 16 - protein, 120 aa, synthetic construct
  REGION 1..120: note = TS00341
EVQLLESGGGLVQPGGSLRLSCAASGFTFSTYWMSWVRQAPGKGLEWVSVINQDGSSTYYADSVKGRFTISRDNSKNTLYLQMNSLRAEDTAVYYCAKDELKSEWDFDYWGQGTLVTVSS

SEQ ID NO: 17 - protein, 124 aa, synthetic construct
  REGION 1..124: note = TS00357
EVQLLESGGGLVQPGGSLRLSCAASGFTFSDYWMSWVRQAPGKGLEWVSNISSSGSSTYYADSVKGRFTISRDNSKNTLYLQMNSLRAEDTAVYYCAKTSDWLSGKIDEHFDYWGQGTLVTVSS

SEQ ID NO: 18 - protein, 119 aa, synthetic construct
  REGION 1..119: note = TS00352
EVQLLESGGGLVQPGGSLRLSCAASGFTFSSYWMHWVRQAPGKGLEWVSAIWSSGGSTYYADSVKGRFTISRDNSKNTLYLQMNSLRAEDTAVYYCAKSKSLHEDFDYWGQGTLVTVSS

SEQ ID NO: 19 - protein, 120 aa, synthetic construct
  REGION 1..120: note = TS00358
EVQLLESGGGLVQPGGSLRLSCAASGFTFSDYWMSWVRQAPGKGLEWVSNISGDGGSTYYADSVKGRFTISRDNSKNTLYLQMNSLRAEDTAVYYCAKTTDLLQGAFDYWGQGTLVTVSS

SEQ ID NO: 20 - protein, 125 aa, synthetic construct
  REGION 1..125: note = TS00362
EVQLLESGGGLVQPGGSLRLSCAASGFTFSDYWMSWVRQAPGKGLEWVSNISSDGGSTYYADSVKGRFTISRDNSKNTLYLQMNSLRAEDTAVYYCAKTTTWLLSQHDKTSFDYWGQGTLVTVSS

SEQ ID NO: 21 - protein, 120 aa, synthetic construct
  REGION 1..120: note = TS00365
EVQLLESGGGLVQPGGSLRLSCAASGFTFSDYWMSWVRQAPGKGLEWVSNINQDGGSTYYADSVKGRFTISRDNSKNTLYLQMNSLRAEDTAVYYCAKTSDWLLVQFDYWGQGTLVTVSS

SEQ ID NO: 22 - protein, 118 aa, synthetic construct
  REGION 1..118: note = TS00344
EVQLLESGGGLVQPGGSLRLSCAASGFTFSTYWMSWVRQAPGKGLEWVSAISGDGSSTYYADSVKGRFTISRDNSKNTLYLQMNSLRAEDTAVYYCAKTWKSPKFDYWGQGTLVTVSS

SEQ ID NO: 23 - protein, 122 aa, synthetic construct
  REGION 1..122: note = TS00372
EVQLLESGGGLVQPGGSLRLSCAASGFTFSSYWMSWVRQAPGKGLEWVSGINYSGSSTYYADSVKGRFTISRDNSKNTLYLQMNSLRAEDTAVYYCAKWSWLGSDKADFDYWGQGTLVTVSS

SEQ ID NO: 24 - protein, 121 aa, synthetic construct
  REGION 1..121: note = TS00374
EVQLLESGGGLVQPGGSLRLSCAASGFTFSTYWMSWVRQAPGKGLEWVSGINQDGSSTYYADSVKGRFTISRDNSKNTLYLQMNSLRAEDTAVYYCAKWHWAGSSQQFDYWGQGTLVTVSS

SEQ ID NO: 25 - protein, 123 aa, synthetic construct
  REGION 1..123: note = TS00379
EVQLLESGGGLVQPGGSLRLSCAASGFTFSSYGMSWVRQAPGKGLEWVSNINQSGSSTYYADSVKGRFTISRDNSKNTLYLQMNSLRAEDTAVYYCAKHSHWAHRVLQSFDYWGQGTLVTVSS

SEQ ID NO: 26 - protein, 120 aa, synthetic construct
  REGION 1..120: note = TS00342
EVQLLESGGGLVQPGGSLRLSCAASGFTFSDYWMSWVRQAPGKGLEWVSNISQSGGSTYYADSVKGRFTISRDNSKNTLYLQMNSLRAEDTAVYYCAKDTPGPRWDFDYWGQGTLVTVSS

SEQ ID NO: 27 - protein, 119 aa, synthetic construct
  REGION 1..119: note = TS00343
EVQLLESGGGLVQPGGSLRLSCAASGFTFSDYWMSWVRQAPGKGLEWVSAIKGDGSSTYYADSVKGRFTISRDNSKNTLYLQMNSLRAEDTAVYYCAKEEWRPYSFDYWGQGTLVTVSS

SEQ ID NO: 28 - protein, 119 aa, synthetic construct
  REGION 1..119: note = TS00345
EVQLLESGGGLVQPGGSLRLSCAASGFTFSNYWMSWVRQAPGKGLEWVSAIKQDGSSTYYADSVKGRFTISRDNSKNTLYLQMNSLRAEDTAVYYCAKEEWRDYDFDYWGQGTLVTVSS

SEQ ID NO: 29 - protein, 120 aa, synthetic construct
  REGION 1..120: note = TS00346
EVQLLESGGGLVQPGGSLRLSCAASGFTFSDYWMSWVRQAPGKGLEWVSAIKQDGGSTYYADSVKGRFTISRDNSKNTLYLQMNSLRAEDTAVYYCAKETWEAHWDFDYWGQGTLVTVSS

SEQ ID NO: 30 - protein, 121 aa, synthetic construct
  REGION 1..121: note = TS00351
EVQLLESGGGLVQPGGSLRLSCAASGFTFSSYAMSWVRQAPGKGLEWVSNISGSGSSTYYADSVKGRFTISRDNSKNTLYLQMNSLRAEDTAVYYCAKESQHSPGWGFDYWGQGTLVTVSS

SEQ ID NO: 31 - protein, 119 aa, synthetic construct
  REGION 1..119: note = TS00355
EVQLLESGGGLVQPGGSLRLSCAASGFTFSDYWMSWVRQAPGKGLEWVSAISGDGSSTYYADSVKGRFTISRDNSKNTLYLQMNSLRAEDTAVYYCAKETWGTYDFDYWGQGTLVTVSS

SEQ ID NO: 32 - protein, 123 aa, synthetic construct
  REGION 1..123: note = TS00361
EVQLLESGGGLVQPGGSLRLSCAASGFTFSNYWMSWVRQAPGKGLEWVSNINGDGSSTYYADSVKGRFTISRDNSKNTLYLQMNSLRAEDTAVYYCAKTTSWLSRQDPDFDYWGQGTLVTVSS

SEQ ID NO: 33 - protein, 124 aa, synthetic construct
  REGION 1..124: note = TS00370
EVQLLESGGGLVQPGGSLRLSCAASGFTFSNYWMSWVRQAPGKGLEWVSNISSDGGSTYYADSVKGRFTISRDNSKNTLYLQMNSLRAEDTAVYYCAKTTTWLTYRQGHGFDYWGQGTLVTVSS

SEQ ID NO: 34 - protein, 122 aa, synthetic construct
  REGION 1..122: note = TS00371
EVQLLESGGGLVQPGGSLRLSCAASGFTFSNYWMSWVRQAPGKGLEWVSGINGSGGSTYYADSVKGRFTISRDNSKNTLYLQMNSLRAEDTAVYYCAKWHWYGQEKKAFDYWGQGTLVTVSS

SEQ ID NO: 35 - protein, 118 aa, synthetic construct
  REGION 1..118: note = TS00377
EVQLLESGGGLVQPGGSLRLSCAASGFTFSDYWMSWVRQAPGKGLEWVSAISGDGSSTYYADSVKGRFTISRDNSKNTLYLQMNSLRAEDTAVYYCAKEDWRPGFDYWGQGTLVTVSS

SEQ ID NO: 36 - protein, 119 aa, synthetic construct
  REGION 1..119: note = TS00378
EVQLLESGGGLVQPGGSLRLSCAASGFTFSNYAMSWVRQAPGKGLEWVSAISGSGGSTYYADSVKGRFTISRDNSKNTLYLQMNSLRAEDTAVYYCAKFPFSHDPFDYWGQGTLVTVSS

SEQ ID NO: 37 - protein, 123 aa, synthetic construct
  REGION 1..123: note = TS00380
EVQLLESGGGLVQPGGSLRLSCAASGFTFSNYWMSWVRQAPGKGLEWVSGISQSGSSTYYADSVKGRFTISRDNSKNTLYLQMNSLRAEDTAVYYCAKWQFASWRGDKDFDYWGQGTLVTVSS

SEQ ID NO: 38 - protein, 121 aa, synthetic construct
  REGION 1..121: note = Cris-7 - VH0
QVQLQQSGAELARPGASVKMSCKASGYTFTRSTMHWVKQRPGQGLEWIGYINPSSAYTNYNQKFKDKATLTADKSSSTAYMQLSSLTSEDSAVYYCASPQVHYDYNGFPYWGQGTLVTVSA

SEQ ID NO: 39 - protein, 121 aa, synthetic construct
  REGION 1..121: note = Cris-7 - VH0
QVQLVQSGAEVKKPGSSVKVSCKASGYTFTRSTMHWVRQAPGQGLEWIGYINPSSAYTNYAQKFQGRATLTADESTSTAYMELSSLRSEDTAVYYCASPQVHYDYNGFPYWGQGTLVTVSS

SEQ ID NO: 40 - protein, 121 aa, synthetic construct
  REGION 1..121: note = Cris-7 - VH2
EVQLVESGGGLVQPGRSLRLSCTASGYTFTRSTMHWVRQAPGKGLEWVGYINPSSAYTNYAASFKGRATLSADGSKSIAYLQMNSLKTEDTAVYYCASPQVHYDYNGFPYWGQGTLVTVSS

SEQ ID NO: 41 - protein, 121 aa, synthetic construct
  REGION 1..121: note = Cris-7 - VH3
QVQLVQSGSELKKPGASVKMSCKASGYTFTRSTMHWVRQAPGQGLEWMGYINPSSAYTNYAQGFTGRAVLSADTSVSTAYLQLCSLKAEDTAVYYCASPQVHYDYNGFPYWGQGTLVTVSS

SEQ ID NO: 42 - protein, 106 aa, synthetic construct
  REGION 1..106: note = cris-7 - VL0
QVVLTQSPAIMSAFPGEKVTMTCSASSSVSYMNWYQQKSGTSPKRWIYDSSKLASGVPARFSGSGSGTSYSLTISSMETEDAATYYCQQWSRNPPTFGGGTKLQIT

SEQ ID NO: 43 - protein, 106 aa, synthetic construct
  REGION 1..106: note = cris-7 - VL1
DIQMTQSPSSLSASVGDRVTITCRASSSVSYMNWYQQKPGKAPKRLIYDSSKLASGVPSRFSGSGSGTEFTLTISSLQPEDFATYYCQQWSRNPPTFGQGTKLEIK

SEQ ID NO: 44 - protein, 106 aa, synthetic construct
  REGION 1..106: note = cris-7 - VL2
AIRLTQSPFSLSASVGDRVTITCSASSSVSYMNWYQQKPAKAPKRWIYDSSKLASGVPSRFSGSGSGTDYTLTISSLQPEDAATYYCQQWSRNPPTFGQGTKLEIK

SEQ ID NO: 45 - protein, 125 aa, synthetic construct
  REGION 1..125: note = SP34 HC
EVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSS

SEQ ID NO: 46 - protein, 109 aa, synthetic construct
  REGION 1..109: note = SP34 LC
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVL

SEQ ID NO: 47 - protein, 15 aa, synthetic construct
  REGION 1..15: note = IgG1 hinge
EPKSCDKTHTCPPCP

SEQ ID NO: 48 - protein, 93 aa, synthetic construct
  REGION 1..93: note = Fibronectin peptide
DQCIVDDITYNVQDTFHKKHEEGHMLNCTCFGQGRGRWKCDPVDQCQDSETGTFYQIGDSWEKYVHGVRYQCYCYGRGIGEWHCQPLQTYPSS

SEQ ID NO: 49 - protein, 23 aa, synthetic construct
  REGION 1..23: note = Collagen peptide 1
GQRGVVGLPGQRGERGFPGLPGY

SEQ ID NO: 50 - protein, 15 aa, synthetic construct
  REGION 1..15: note = Collagen peptide 2
GQRGVVGLPGQRGER

SEQ ID NO: 51 - protein, 9 aa, synthetic construct
  REGION 1..9: note = Anti-CD3 antibody shield
QDGNEEMGG

SEQ ID NO: 52 - protein, 6 aa, synthetic construct
  REGION 1..6: note = Substrate sequence for MMP2 and MMP9
GPLGVR

SEQ ID NO: 53 - protein, 6 aa, synthetic construct
  REGION 1..6: note = Substrate sequence for MMP2 and MMP9
PLGLAR

SEQ ID NO: 54 - protein, 6 aa, synthetic construct
  REGION 1..6: note = Substrate sequence for MMP2 and MMP9
PLGLAG

SEQ ID NO: 55 - protein, 8 aa, synthetic construct
  REGION 1..8: note = Substrate sequence for MMP2 and MMP9
IPVSLRSG

SEQ ID NO: 56 - protein, 8 aa, synthetic construct
  REGION 1..8: note = Substrate sequence for MMP2 and MMP9
GPLGMLSQ

SEQ ID NO: 57 - protein, 10 aa, synthetic construct
  REGION 1..10: note = Substrate sequence for MMP3
RPKPVEVWRK

SEQ ID NO: 58 - protein, 8 aa, synthetic construct
  REGION 1..8: note = Substrate sequence for uPA
LSGRSDNH

SEQ ID NO: 59 - protein, 8 aa, synthetic construct
  REGION 1..8: note = Substrate sequence for uPA
TGRGPSWV

SEQ ID NO: 60-61 - skipped (2 entries)

SEQ ID NO: 62 - protein, 17 aa, synthetic construct
  REGION 1..17: note = CDH17 LC1 CDR1
KSSQSLLHSSNQKNYLA

SEQ ID NO: 63 - protein, 7 aa, synthetic construct
  REGION 1..7: note = CDH17 LC1 CDR2
WASTRES

SEQ ID NO: 64 - protein, 9 aa, synthetic construct
  REGION 1..9: note = CDH17 LC1 CDR3
QQYYSYPWT

SEQ ID NO: 65 - protein, 13 aa, synthetic construct
  REGION 1..13: note = CDH17 HC1 CDR1
KVSGYTLTDHTIH

SEQ ID NO: 66 - protein, 7 aa, synthetic construct
  REGION 1..7: note = CDH17 HC1 CDR2
GYNEKFK

SEQ ID NO: 67 - protein, 16 aa, synthetic construct
  REGION 1..16: note = CDH17 HC1 CDR3
ARWGYSYRNYAYYYDY

SEQ ID NO: 68 - protein, 13 aa, synthetic construct
  REGION 1..13: note = CDH17 E2 HC CDR1
KVSGYTFTDHTIH

SEQ ID NO: 69 - protein, 7 aa, synthetic construct
  REGION 1..7: note = CDH17 E2 HC CDR2
QYNEKFK

SEQ ID NO: 70 - protein, 16 aa, synthetic construct
  REGION 1..16: note = CDH17 E2 HC CDR3
ARWGYFYGSRSYYFDY

SEQ ID NO: 71 - protein, 11 aa, synthetic construct
  REGION 1..11: note = CDH17 E2 LC CDR1
KASQNVGTAVA

SEQ ID NO: 72 - protein, 7 aa, synthetic construct
  REGION 1..7: note = CDH17 E2 LC CDR2
GVPDRFT

SEQ ID NO: 73 - protein, 9 aa, synthetic construct
  REGION 1..9: note = CDH17 E2 LC CDR3
QQYSSYPWT

SEQ ID NO: 74 - protein, 5 aa, synthetic construct
  REGION 1..5: note = TS00349 CDR1
DYWMH

SEQ ID NO: 75 - protein, 17 aa, synthetic construct
  REGION 1..17: note = TS00349, TS00352 CDR2
AIWSSGGSTYYADSVKG

SEQ ID NO: 76 - protein, 10 aa, synthetic construct
  REGION 1..10: note = TS00349 CDR3
SPTLYDDFDY

SEQ ID NO: 77 - protein, 5 aa, synthetic construct
  REGION 1..5: note = TS00369, TS00366, TS00364, TS00357, TS00358, TS00362, TS00365, TS00342, TS00343, TS00346, TS00355, TS00377 CDR1
DYWMS

SEQ ID NO: 78 - protein, 17 aa, synthetic construct
  REGION 1..17: note = TS00369, TS00351 CDR2
NISGSGSSTYYADSVKG

SEQ ID NO: 79 - protein, 15 aa, synthetic construct
  REGION 1..15: note = TS00369 CDR3
TSQWLARERSSEFDY

SEQ ID NO: 80 - protein, 5 aa, synthetic construct
  REGION 1..5: note = TS00347 CDR1
TYAMS

SEQ ID NO: 81 - protein, 17 aa, synthetic construct
  REGION 1..17: note = TS00347, TS00364 CDR2
NISGSGGSTYYADSVKG

SEQ ID NO: 82 - protein, 12 aa, synthetic construct
  REGION 1..12: note = TS00347 CDR3
EQQWVPGFGFDY

SEQ ID NO: 83 - protein, 17 aa, synthetic construct
  REGION 1..17: note = TS00366 CDR2
NINGDGGSTYYADSVKG

SEQ ID NO: 84 - protein, 12 aa, synthetic construct
  REGION 1..12: note = TS00366 CDR3
TSTFYKQDKFDY

SEQ ID NO: 85 - protein, 15 aa, synthetic construct
  REGION 1..15: note = TS00364 CDR3
TSDWLLHDADLHFDY

SEQ ID NO: 86 - protein, 5 aa, synthetic construct
TYWMS

SEQ ID NO: 87 - protein, 17 aa, synthetic construct
  REGION 1..17: note = TS00363 CDR2
NISQDGGSTYYADSVKG

SEQ ID NO: 88 - protein, 15 aa, synthetic construct
  REGION 1..15: note = TS00363 CDR3
TTDWLQDKVDEAFDY

SEQ ID NO: 89 - protein, 17 aa, synthetic construct
  REGION 1..17: note = TS00341 CDR2
VINQDGSSTYYADSVKG

SEQ ID NO: 90 - protein, 11 aa, synthetic construct
  REGION 1..11: note = TS00341 CDR3
DELKSEWDFDY

SEQ ID NO: 91 - protein, 17 aa, synthetic construct
  REGION 1..17: note = TS00357 CDR2
NISSSGSSTYYADSVKG

SEQ ID NO: 92 - protein, 15 aa, synthetic construct
  REGION 1..15: note = TS00357 CDR3
TSDWLSGKIDEHFDY

SEQ ID NO: 93 - protein, 5 aa, synthetic construct
  REGION 1..5: note = TS00352 CDR1
SYWMH

SEQ ID NO: 94 - protein, 10 aa, synthetic construct
  REGION 1..10: note = TS00352 CDR3
SKSLHEDFDY

SEQ ID NO: 95 - protein, 17 aa, synthetic construct
  REGION 1..17: note = TS00358 CDR2
NISGDGGSTYYADSVKG

SEQ ID NO: 96 - protein, 11 aa, synthetic construct
  REGION 1..11: note = TS00358 CDR3
TTDLLQGAFDY

SEQ ID NO: 97 - protein, 17 aa, synthetic construct
  REGION 1..17: note = TS00362 CDR2
NISSDGGSTYYADSVKG

SEQ ID NO: 98 - protein, 16 aa, synthetic construct
  REGION 1..16: note = TS00362 CDR3
TTTWLLSQHDKTSFDY

SEQ ID NO: 99 - protein, 17 aa, synthetic construct
  REGION 1..17: note = TS00365 CDR2
NINQDGGSTYYADSVKG

SEQ ID NO: 100 - protein, 11 aa, synthetic construct
  REGION 1..11: note = TS00365 CDR3
TSDWLLVQFDY

SEQ ID NO: 101 - protein, 17 aa, synthetic construct
  REGION 1..17: note = TS00344, TS00355, TS00377 CDR2
AISGDGSSTYYADSVKG

SEQ ID NO: 102 - protein, 9 aa, synthetic construct
  REGION 1..9: note = TS00344 CDR3
TWKSPKFDY

SEQ ID NO: 103 - protein, 5 aa, synthetic construct
  REGION 1..5: note = TS00372 CDR1
SYWMS

SEQ ID NO: 104 - protein, 17 aa, synthetic construct
  REGION 1..17: note = TS00372 CDR2
GINYSGSSTYYADSVKG

SEQ ID NO: 105 - protein, 13 aa, synthetic construct
  REGION 1..13: note = TS00372 CDR3
WSWLGSDKADFDY

SEQ ID NO: 106 - protein, 17 aa, synthetic construct
  REGION 1..17: note = TS00374 CDR2
GINQDGSSTYYADSVKG

SEQ ID NO: 107 - protein, 12 aa, synthetic construct
  REGION 1..12: note = TS00374 CDR3
WHWAGSSQQFDY

SEQ ID NO: 108 - protein, 5 aa, synthetic construct
  REGION 1..5: note = TS00379 CDR1
SYGMS

SEQ ID NO: 109 - protein, 17 aa, synthetic construct
  REGION 1..17: note = TS00379 CDR2
NINQSGSSTYYADSVKG

SEQ ID NO: 110 - protein, 14 aa, synthetic construct
  REGION 1..14: note = TS00379 CDR3
HSHWAHRVLQSFDY

SEQ ID NO: 111 - protein, 17 aa, synthetic construct
  REGION 1..17: note = TS00342 CDR2
NISQSGGSTYYADSVKG

SEQ ID NO: 112 - protein, 11 aa, synthetic construct
  REGION 1..11: note = TS00342 CDR3
DTPGPRWDFDY

SEQ ID NO: 113 - protein, 17 aa, synthetic construct
  REGION 1..17: note = TS00343 CDR2
AIKGDGSSTYYADSVKG

SEQ ID NO: 114 - protein, 10 aa, synthetic construct
  REGION 1..10: note = TS00343 CDR3
EEWRPYSFDY

SEQ ID NO: 115 - protein, 5 aa, synthetic construct
  REGION 1..5: note = TS00345, TS00361, TS00370, TS00371, TS00380 CDR1
NYWMS

SEQ ID NO: 116 - protein, 17 aa, synthetic construct
  REGION 1..17: note = TS00345 CDR2
AIKQDGSSTYYADSVKG

SEQ ID NO: 117 - protein, 10 aa, synthetic construct
  REGION 1..10: note = TS00345 CDR3
EEWRDYDFDY

SEQ ID NO: 118 - protein, 17 aa, synthetic construct
  REGION 1..17: note = TS00346 CDR2
AIKQDGGSTYYADSVKG

SEQ ID NO: 119 - protein, 11 aa, synthetic construct
  REGION 1..11: note = TS00346 CDR3
ETWEAHWDFDY

SEQ ID NO: 120 - protein, 5 aa, synthetic construct
  REGION 1..5: note = TS00351 CDR1
SYAMS

SEQ ID NO: 121 - protein, 12 aa, synthetic construct
  REGION 1..12: note = TS00351 CDR3
ESQHSPGWGFDY

SEQ ID NO: 122 - protein, 10 aa, synthetic construct
  REGION 1..10: note = TS00355 CDR3
ETWGTYDFDY

SEQ ID NO: 123 - protein, 17 aa, synthetic construct
  REGION 1..17: note = TS00361 CDR2
NINGDGSSTYYADSVKG

SEQ ID NO: 124 - protein, 14 aa, synthetic construct
  REGION 1..14: note = TS00361 CDR3
TTSWLSRQDPDFDY

SEQ ID NO: 125 - protein, 17 aa, synthetic construct
  REGION 1..17: note = TS00370 CDR2
NISSDGGSTYYADSVKG

SEQ ID NO: 126 - protein, 15 aa, synthetic construct
  REGION 1..15: note = TS00370 CDR3
TTTWLTYRQGHGFDY

SEQ ID NO: 127 - protein, 17 aa, synthetic construct
  REGION 1..17: note = TS00371 CDR2
GINGSGGSTYYADSVKG

SEQ ID NO: 128 - protein, 13 aa, synthetic construct
  REGION 1..13: note = TS00371 CDR3
WHWYGQEKKAFDY

SEQ ID NO: 129 - protein, 9 aa, synthetic construct
  REGION 1..9: note = TS00377 CDR3
EDWRPGFDY

SEQ ID NO: 130 - protein, 5 aa, synthetic construct
  REGION 1..5: note = TS00378 CDR1
NYAMS

SEQ ID NO: 131 - protein, 17 aa, synthetic construct
  REGION 1..17: note = TS00378 CDR2
AISGSGGSTYYADSVKG

SEQ ID NO: 132 - protein, 10 aa, synthetic construct
  REGION 1..10: note = TS00378 CDR3
FPFSHDPFDY

SEQ ID NO: 133 - protein, 17 aa, synthetic construct
  REGION 1..17: note = TS00380 CDR2
GISQSGSSTYYADSVKG

SEQ ID NO: 134 - protein, 14 aa, synthetic construct
  REGION 1..14: note = TS00380 CDR3
WQFASWRGDKDFDY

SEQ ID NO: 135 - protein, 5 aa, synthetic construct
  REGION 1..5: note = Cris-7 - VH1, Cris-7 - VH2, Cris-7 - VH3 CDR1
RSTMH

SEQ ID NO: 136 - protein, 17 aa, synthetic construct
  REGION 1..17: note = Cris-7 - VH1 CDR2
YINPSSAYTNYAQKFQG

SEQ ID NO: 137 - protein, 15 aa, synthetic construct
  REGION 1..15: note = Cris-7 - VH1, Cris-7 - VH2, Cris-7 - VH3 CDR3
CDRPQVHYDYNGFPY

SEQ ID NO: 138 - protein, 17 aa, synthetic construct
  REGION 1..17: note = Cris-7 - VH2 CDR2
YINPSSAYTNYAASFKG

SEQ ID NO: 139 - protein, 17 aa, synthetic construct
  REGION 1..17: note = Cris-7 - VH3 CDR2
YINPSSAYTNYAQGFTG

SEQ ID NO: 140 - protein, 10 aa, synthetic construct
  REGION 1..10: note = cris-7 - VL1 CDR1
RASSSVSYMN

SEQ ID NO: 141 - protein, 7 aa, synthetic construct
  REGION 1..7: note = cris-7 - VL1, cris-7 - VL2 CDR2
DSSKLAS

SEQ ID NO: 142 - protein, 9 aa, synthetic construct
  REGION 1..9: note = cris-7 - VL1, cris-7 - VL2 CDR3
QQWSRNPPT

SEQ ID NO: 143 - protein, 10 aa, synthetic construct
  REGION 1..10: note = cris-7 - VL2 CDR1
SASSSVSYMN

SEQ ID NO: 144 - protein, 5 aa, synthetic construct
  REPEAT 1..5: note = (GGGGS)4 linker
GGGGS

SEQ ID NO: 145 - protein, 123 aa, synthetic construct
  REGION 1..123: note = TS00376
EVQLLESGGGLVQPGGSLRLSCAASGFTFSDYWMSWVRQAPGKGLEWVSGINSSGGSTYYADSVKGRFTISRDNSKNTLYLQMNSLRAEDTAVYYCAKWQFVTQPGHKAFDYWGQGTLVTVSS

SEQ ID NO: 146 - protein, 17 aa, synthetic construct
  REGION 1..17: note = TS00376 CDR2
GINSSGGSTYYADSVKG

SEQ ID NO: 147 - protein, 14 aa, synthetic construct
  REGION 1..14: note = TS00376 CDR3
WQFVTQPGHKAFDY